(19) United States
(12) Patent Application Publication
(10) Pub. No.: US 2015/0250172 A1
(43) Pub. Date: Sep. 10, 2015

(54) USE OF ANTHRANILAMIDE COMPOUNDS IN SOIL AND SEED TREATMENT APPLICATION METHODS

(71) Applicant: BASF SE, Ludwigshafen (DE)

(72) Inventors: Karsten Körber, Eppelheim (DE); Florian Kaiser, Mannheim (DE); Prashant Deshmukh, Mannheim (DE); Matthias Pohlman, Freinsheim (DE); Jean-Yves Wach, Mannheim (DE); Deborah L. Culbertson, Fuquay Varina, NC (US); W. David Rogers, Durham, NC (US); Michael David, Raleigh, NC (US); Franz Josef Braun, Durham, NC (US); Sarah Thompson, Raleigh, NC (US)

(73) Assignee: BASF SE, Ludwigshafen (DE)

(21) Appl. No.: 14/431,974

(22) PCT Filed: Sep. 27, 2013
(86) PCT No.: PCT/EP2013/070147

§ 371 Date: Mar. 27, 2015

Related U.S. Application Data
(60) Provisional application No. 61/708,067, filed on Oct. 1, 2012.
(60) Provisional application No. 61/763,966, filed on Feb. 13, 2013.

(51) Int. Cl. A01N 43/56; C07D 401/04

(52) U.S. Cl. CPC A01N 43/56; C07D 401/04

(57) ABSTRACT

The present invention relates to agricultural methods using anthranilamide compounds of formula (I)

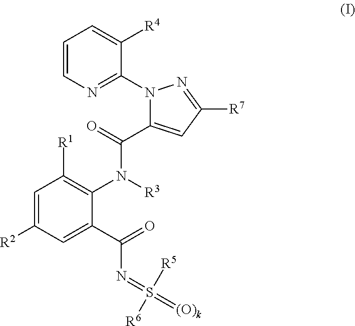

wherein R¹, R², R³, R⁴, R⁵, R⁶, R⁷ and k are as defined in the description;
and their mixtures;
for controlling and/or combating animal pests in soil application methods and seed treatment methods

The anthranilamide compounds of formula (I) are highly suitable for controlling animal pests such as insects and/or spider mites and/or nematodes by treating the soil/growth substrate by drenching or drip application or dipping or soil injection.

The present invention relates to new uses of N-thio-anthranilamide compounds, and their mixtures with selected other pesticides in soil and seed treatment application methods.

Invertebrate pests, arthropods and nematodes, and in particular insects and arachnids, destroy growing and harvested crops and attack wooden dwelling and commercial structures, thereby causing large economic loss to the food supply and to property. While a large number of pesticidal agents are known, due to the ability of target pests to develop resistance to said agents, there is an ongoing need for new agents for combating invertebrate pests such as insects, arachnids and nematodes.

Especially soil-living pests, arthropod pests, including soil-living insects and arachnids, and especially spider mites, and nematodes, are often controlled and combated by applying an effective amount of a suitable pesticide compound to the soil, e.g. by drenching, drip application, dip application or soil injection. The pesticidal compounds may further be applied as a solid or liquid composition, e.g. such as a dust or granule formulation comprising an inert carrier, e.g. such as clay.

Methods of soil application can suffer from several problems. Pesticidal compounds are not always especially suitable for being applied by different soil application methods such as by drenching, drip application, dip application or soil injection. Their pesticidal activity may be affected in some cases.

It is therefore an object of the present invention to provide compounds having a good pesticidal activity and a good applicability in techniques of soil treatment against a large number of different invertebrate pests, especially against soil-living pests, which are difficult to control.

Some soil-applied pesticides compositions may also have potential for leaching. Therefore, care must be taken to minimize both surface and ground water contamination. Moreover, the effectiveness of the pesticide may vary depending on environmental conditions—e.g. properly timed rain is needed for the successful functioning of the chemistry in the soil, but too much rain may reduce the effectiveness and may cause leaching.

It is therefore also an object of the present invention to provide compositions which are suitable for combating soil-living pests and which overcome the problems associated with the known techniques. In particular the compositions should be applicable easily and provide a long-lasting action on soil-living pests. Moreover, environmental conditions should not have an adverse effect on the effectiveness of the pesticide.

It is therefore also an object of the present invention to provide methods of application, which are suitable for combating soil-living pests

Soil application methods are considered as different techniques of applying pesticidal compounds directly or indirectly to the soil and/or ground, such as drip applications or drip irrigations (onto the soil), or soil injection, further methods of drenching the soil.

Furthermore, object of the present inventions are methods of application by dipping roots, tubers or bulbs (referred to as dip application), by hydroponic systems or also by seed treatment.

Another of the problems the farmer is faced with in this context is, that seeds and plant roots and shoots are constantly threatened by foliar and soil insects and other pests.

Thus a further difficulty in relation to the use of such seed protection pesticides is that the repeated and exclusive application of an individual pesticidal compound leads also here in many cases to a rapid selection of soil pests, which have developed natural or adapted resistance against the active compound in question. Therefore there is a need for seed protection agents that help prevent or overcome resistance.

It is therefore a further object of the present invention to provide compounds which solve the problems of protection of the protection of seeds and growing plants, reducing the dosage rate, enhancing the spectrum of activity and/or to manage pest resistance.

The present invention therefore also provides methods for the protection of plant propagation material, especially seeds, from soil insects and of the resulting plant's roots and shoots from soil and foliar insects.

The invention also relates to plant propagation material, especially seeds, which is protected from soil and foliar insects.

Surprisingly, it has now been found that anthranilamide compounds of formula (I):

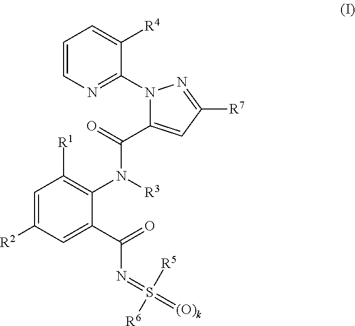

wherein
R¹ is selected from the group consisting of halogen, methyl and halomethyl;
R² is selected from the group consisting of hydrogen, halogen, halomethyl and cyano;
R³ is selected from hydrogen, C₁-C₆ alkyl, C₁-C₆ haloalkyl, C₂-C₆-alkenyl, C₂-C₆-haloalkenyl, C₂-C₆-alkinyl, C₂-C₆-haloalkinyl, C₃-C₈-cycloalkyl, C₃-C₈-halocycloalkyl, C₁-C₄-alkoxy-C₁-C₄-alkyl, C₁-C₄-haloalkoxy-C₁-C₄-alkyl, C(═O)R^{a}, C(═O)OR^{b} and C(═O)NR^{c}R^{d};
R⁴ is hydrogen or halogen;
R⁵, R⁶ are selected independently of one another from the group consisting of hydrogen, C₁-C₁₀-alkyl, C₃-C₈-cycloalkyl, C₂-C₁₀-alkenyl, C₂-C₁₀-alkynyl, wherein the aforementioned aliphatic and cycloaliphatic radicals may be substituted with 1 to 10 substituents R^{e}, and phenyl, which is unsubstituted or carries 1 to 5 substituents R^{f}; or
   R⁵ and R⁶ together represent a C₂-C₇-alkylene, C₂-C₇-alkenylene or C₆-C₉-alkynylene chain forming together with the sulfur atom to which they are attached a 3-, 4-, 5-, 6-, 7-, 8-, 9- or 10-membered saturated, partially unsaturated or fully unsaturated ring, wherein 1 to 4 of the CH₂ groups in the C₂-C₇-alkylene chain or 1 to 4 of any of the CH₂ or CH groups in the C₂-C₇-alkenylene chain or 1 to 4 of any of the CH₂ groups in the C₆-C₉-alkynylene chain may be replaced by 1 to 4 groups independently selected from the group consisting of C═O, C═S, O, S, N, NO, SO, SO₂ and NH, and wherein the carbon and/or nitrogen atoms in the C₂-C₇-alkylene, C₂-C₇-alkenylene or C₆-C₉-alkynylene chain may be substituted with 1 to 5 substituents independently selected from the group consisting of halogen, cyano, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkynyl and C₂-C₆-haloalkynyl; said substituents being identical or different from one another if more than one substituent is present;
R⁷ is selected from the group consisting of bromo, chloro, difluoromethyl, trifluoromethyl, nitro, cyano, OCH₃, OCHF₂, OCH₂F, OCH₂CF₃, S(═O)ₙCH₃, and S(═O)ₙCF₃;
R^{a} is selected from the group consisting of C₁-C₆-alkyl, C₂-C₆-alkenyl, C₂-C₆-alkinyl, C₃-C₈-cycloalkyl, C₁-C₆-alkoxy, C₁-C₆-alkylthio, C₁-C₆-alkylsulfinyl, C₁-C₆-alkylsulfonyl, wherein one or more CH₂ groups of the aforementioned radicals may be replaced by a C═O group, and/or the aliphatic and cycloaliphatic moieties of the aforementioned radicals may be unsubstituted, partially or fully halogenated and/or may carry 1 or 2 substituents selected from C₁-C₄ alkoxy;
   phenyl, benzyl, pyridyl and phenoxy, wherein the last four radicals may be unsubstituted, partially or fully halogenated and/or carry 1, 2 or 3 substituents selected from C₁-C₆-alkyl, C₁-C₆-haloalkyl, C₁-C₆-alkoxy, C₁-C₆-haloalkoxy, (C₁-C₆-alkoxy)carbonyl, C₁-C₆-alkylamino and di-(C₁-C₆-alkyl)amino,
R^{b} is selected from the group consisting of C₁-C₆-alkyl, C₂-C₆-alkenyl, C₂-C₆-alkinyl, C₃-C₈-cycloalkyl, C₁-C₆-alkoxy, C₁-C₆-alkylthio, C₁-C₆-alkylsulfinyl, C₁-C₆-alkylsulfonyl, wherein one or more CH₂ groups of the aforementioned radicals may be replaced by a C═O group, and/or the aliphatic and cycloaliphatic moieties of the aforementioned radicals may be unsubstituted, partially or fully halogenated and/or may carry 1 or 2 substituents selected from C₁-C₄-alkoxy;
   phenyl, benzyl, pyridyl and phenoxy, wherein the last four radicals may be unsubstituted, partially or fully halogenated and/or carry 1, 2 or 3 substituents selected from C₁-C₆-alkyl, C₁-C₆-haloalkyl, C₁-C₆-alkoxy, C₁-C₆-haloalkoxy and (C₁-C₆-alkoxy)carbonyl;
R^{c}, R^{d} are, independently from one another and independently of each occurrence, selected from the group consisting of hydrogen, cyano, C₁-C₆-alkyl, C₂-C₆-alkenyl, C₂-C₆-alkinyl, C₃-C₈-cycloalkyl, wherein one or more CH₂ groups of the aforementioned radicals may be replaced by a C═O group, and/or the aliphatic and cycloaliphatic moieties of the aforementioned radicals may be unsubstituted, partially or fully halogenated and/or may carry 1 or 2 radicals selected from C₁-C₄-alkoxy; C₁-C₆-alkoxy, C₁-C₆-haloalkoxy, C₁-C₆-alkylthio, C₁-C₆-alkylsulfinyl, C₁-C₆-alkylsulfonyl, C₁-C₆-haloalkylthio, phenyl, benzyl, pyridyl and phenoxy, wherein the four last mentioned radicals may be unsubstituted, partially or fully halogenated and/or carry 1, 2 or 3 substituents selected from C₁-C₆-alkyl, C₁-C₆-haloalkyl, C₁-C₆-alkoxy, C₁-C₆ haloalkoxy and (C₁-C₆-alkoxy)carbonyl; or
   R^{c} and R^{d}, together with the nitrogen atom to which they are bound, may form a 3-, 4-, 5-, 6- or 7-membered saturated, partially unsaturated or fully unsaturated heterocyclic ring which may additionally contain 1 or 2 further heteroatoms or heteroatom groups selected from N, O, S, NO, SO and SO₂, as ring members, where the heterocyclic ring may optionally be substituted with halogen, C₁-C₄-haloalkyl, C₁-C₄-alkoxy or C₁-C₄-haloalkoxy;
R^{e} is independently selected from the group consisting of halogen, cyano, nitro, —OH, —SH, —SCN, C₁-C₆-alkyl, C₂-C₆-alkenyl, C₂-C₆-alkinyl, C₃-C₈-cycloalkyl, wherein one or more CH₂ groups of the aforementioned radicals may be replaced by a C═O group, and/or the aliphatic and cycloaliphatic moieties of the aforementioned radicals may be unsubstituted, partially or fully halogenated and/or may carry 1 or 2 radicals selected from C₁-C₄ alkoxy;
   C₁-C₆-alkoxy, C₁-C₆-haloalkoxy, C₁-C₆-alkylthio, C₁-C₆-alkylsulfinyl, C₁-C₆-alkylsulfonyl, C₁-C₆-haloalkylthio, —OR^{a}, —NR^{c}R^{d}, —S(O)ₙR^{a}, —S(O)ₙNR^{c}R^{d}, —C(═O)R^{a}, —C(═O)NR^{c}R^{d}, —C(═O)OR^{b}, —C(═S)R^{a}, —C(═S)NR^{c}R^{d}, —C(═S)OR^{b}, —C(═S)SR^{b}, —C(═NR^{c})R^{b}, —C(═NR^{c})NR^{c}R^{d}, phenyl, benzyl, pyridyl and phenoxy, wherein the last four radicals may be unsubstituted, partially or fully halogenated and/or carry 1, 2 or 3 substituents selected from C₁-C₆-alkyl, C₁-C₆-haloalkyl, C₁-C₆-alkoxy and C₁-C₆-haloalkoxy; or
   two vicinal radicals R^{e} together form a group ═O, ═CH(C₁-C₄-alkyl), ═C(C₁-C₄-alkyl)C₁-C₄-alkyl, ═N(C₁-C₆-alkyl) or ═NO(C₁-C₆-alkyl);
R^{f} is independently selected from the group consisting of halogen, cyano, nitro, —OH, —SH, —SCN, C₁-C₆-alkyl, C₂-C₆-alkenyl, C₂-C₆-alkinyl, C₃-C₈-cycloalkyl, wherein one or more CH₂ groups of the aforementioned radicals may be replaced by a C═O group, and/or the aliphatic and cycloaliphatic moieties of the aforementioned radicals may be unsubstituted, partially or fully halogenated and/or may carry 1 or 2 radicals selected from C₁-C₄ alkoxy;
   C₁-C₆-alkoxy, C₁-C₆-haloalkoxy, C₁-C₆-alkylthio, C₁-C₆-alkylsulfinyl, C₁-C₆-alkylsulfonyl, C₁-C₆-haloalkylthio, —OR^{a}, —NR^{c}R^{d}, —S(O)ₙR^{a}, —S(O)ₙNR^{c}R^{d}, —C(═O)R^{a}, —C(═O)NR^{c}R^{d}, —C(═O)OR^{b}, —C(═S)R^{a}, —C(═S)NR^{c}R^{d}, —C(═S)OR^{b}, —C(═S)SR^{b}, —C(═NR^{c})R^{b}, and —C(═NR^{c})NR^{c}R^{d};
k is 0 or 1;
n is 0, 1 or 2;
or a stereoisomer, salt, tautomer or N-oxide, or a polymorphic crystalline form, a co-crystal
or a solvate of a compound or a stereoisomer, salt, tautomer or N-oxide thereof;
   are, alone or in combination with other selected pesticidal compounds (II), highly suitable for addressing such needs in agriculture. It has been found that these objects as mentioned above are in part or in whole achieved by soil application techniques and seed treatment methods for the control, the protection and the combat from soil insects. This relates especially to seeds and to the resulting plant's roots and shoots.

Thus, in one embodiment, the invention relates to the use of these compounds of formula I (or a stereoisomer, salt, tautomer or N-oxide, or a polymorphic crystalline form, a co-crystal or a solvate of a compound or a stereoisomer, salt, tautomer or N-oxide thereof) for controlling and/or combating animal pests in soil application methods and seed treatment methods.

In one embodiment, the invention relates to said use for controlling and/or combating animal pests in soil application methods and seed treatment methods.

Furthermore, in this context, it has also been found that the compounds of formula I and their mixtures with other pesticides, are especially suitable for the protection of seeds from soil insects and of the resulting plant's roots and shoots from soil and foliar insects.

In seed and soil treatment, there are certain pests which represent a big threat to plants during the stage from shoot/seedling to a small plant. Some pests which are especially known to represent a risk for the shoot/seedling or small plant, include rootworms, wireworms (e.g. in potatoe crop protection) and maggots like seedcorn maggot (e.g. Delia platura), western corn rootworm, black cutworm, mites, spider mites. These are only some examples; there are more pests specifically interesting in seed and soil treatment, which the person skilled in the art knows. Thus, the compounds of formula I or their agriculturally acceptable salts, and/or their mixtures with other selected pesticides, are highly suitable for methods for controlling and/or combating insects, acarids and/or nematodes, and especially spider mites, by soil application methods. According to the present invention, the compounds of formula I and/or their mixtures are used for controlling arthropods, especially insects and arachnids, more especially (spider) mites, and/or nematodes by soil application methods such as drenching, drip application, dip application or soil injection or by seed treatment.

Seed treatment methods comprise e.g contacting the seeds before sowing and/or after pregermination with comprising the compounds of formula I and their mixtures with other pesticides.

### Compounds of Formula I

WO 2007/006670, describes N-thio-anthranilamide compounds with a sulfilimine or sulfoximine group and their use as pesticides. PCT/EP2012/065650, PCT/EP2012/065651, and the unpublished applications U.S. 61/578,267, U.S. 61/593,897 and U.S. 61/651,050 describe certain N-Thio-anthranilamide compounds and their use as pesticides.

PCT/EP2012/065648, PCT/EP2012/065649 and EP11189973.8 describe processes for the synthesis of N-Thio-anthranilamide compounds.

However, although the anthranilamide compounds of formula (I) themselves and their combined application with other insecticides are known to have shown activity against certain crop damaging insect pests, the compounds of formula I and some of their selected mixtures with pesticidally active compounds (II) have not yet been described for solving discussed problems as mentioned above.

Especially, their surprisingly excellent applicability for soil application techniques as well as seed treatment, and their extraordinary activity against soil-living pests have not been described previously.

The organic moieties mentioned in the above definitions of the variables are—like the term halogen—collective terms for individual listings of the individual group members. The prefix Cₙ-Cₘ indicates in each case the possible number of carbon atoms in the group.

The term halogen denotes in each case fluorine, bromine, chlorine or iodine, in particular fluorine, chlorine or bromine.

The term “partially or fully halogenated” will be taken to mean that 1 or more, e.g. 1, 2, 3, 4 or 5 or all of the hydrogen atoms of a given radical have been replaced by a halogen atom, in particular by fluorine or chlorine. A partially or fully halogenated radical is termed below also “haloradical”. For example, partially or fully halogenated alkyl is also termed haloalkyl.

The term “alkyl” as used herein (and in the alkyl moieties of other groups comprising an alkyl group, e.g. alkoxy, alkylcarbonyl, alkylthio, alkylsulfinyl, alkylsulfonyl and alkoxyalkyl) denotes in each case a straight-chain or branched alkyl group having usually from 1 to 12 or 1 to 10 carbon atoms, frequently from 1 to 6 carbon atoms, preferably 1 to 4 carbon atoms and in particular from 1 to 3 carbon atoms. Examples of C₁-C₄-alkyl are methyl, ethyl, n-propyl, isopropyl, n-butyl, 2-butyl (sec-butyl), isobutyl and tert-butyl. Examples for C₁-C₆-alkyl are, apart those mentioned for C₁-C₄-alkyl, n-pentyl, 1-methylbutyl, 2-methylbutyl, 3-methylbutyl, 2,2-dimethylpropyl, 1-ethylpropyl, n-hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl. Examples for C₁-C₁₀-alkyl are, apart those mentioned for C₁-C₆-alkyl, n-heptyl, 1-methylhexyl, 2-methylhexyl, 3-methylhexyl, 4-methylhexyl, 5-methylhexyl, 1-ethylpentyl, 2-ethylpentyl, 3-ethylpentyl, n-octyl, 1-methyloctyl, 2-methylheptyl, 1-ethylhexyl, 2-ethylhexyl, 1,2-dimethylhexyl, 1-propylpentyl, 2-propylpentyl, nonyl, decyl, 2-propylheptyl and 3-propylheptyl.

The term “alkylene” (or alkanediyl) as used herein in each case denotes an alkyl radical as defined above, wherein one hydrogen atom at any position of the carbon backbone is replaced by one further binding site, thus forming a bivalent moiety.

The term “haloalkyl” as used herein (and in the haloalkyl moieties of other groups comprising a haloalkyl group, e.g. haloalkoxy, haloalkylthio, haloalkylcarbonyl, haloalkylsulfonyl and haloalkylsulfinyl) denotes in each case a straight-chain or branched alkyl group having usually from 1 to 10 carbon atoms (“C₁-C₁₀-haloalkyl”), frequently from 1 to 6 carbon atoms (“C₁-C₆-haloalkyl”), more frequently 1 to 4 carbon atoms (“C₁-C₁₀-haloalkyl”), wherein the hydrogen atoms of this group are partially or totally replaced with halogen atoms. Preferred haloalkyl moieties are selected from C₁-C₄-haloalkyl, more preferably from C₁-C₂-haloalkyl, more preferably from halomethyl, in particular from C₁-C₂-fluoroalkyl. Halomethyl is methyl in which 1, 2 or 3 of the hydrogen atoms are replaced by halogen atoms. Examples are bromomethyl, chloromethyl, dichloromethyl, trichloromethyl, fluoromethyl, difluoromethyl, trifluoromethyl, chlorofluoromethyl, dichlorofluoromethyl, chlorodifluoromethyl and the like. Examples for C₁-C₂-fluoroalkyl are fluoromethyl, difluoromethyl, trifluoromethyl, 1-fluoroethyl, 2-fluoroethyl, 2,2-difluoroethyl, 2,2,2-trifluoroethyl, pentafluoroethyl, and the like. Examples for C₁-C₂-haloalkyl are, apart those mentioned for C₁-C₂-fluoroalkyl, chloromethyl, dichloromethyl, trichloromethyl, bromomethyl, chlorofluoromethyl, dichlorofluoromethyl, chlorodifluoromethyl, 1-chloroethyl, 2-chloroethyl, 2,2,-dichloroethyl, 2,2,2-trichloroethyl, 2-chloro-2-fluoroethyl, 2-chloro-2,2-difluoroethyl, 2,2-dichloro-2-fluoroethyl, 1-bromoethyl, and the like. Examples for C₁-C₄-haloalkyl are, apart those mentioned for C₁-C₂-haloalkyl, 1-fluoropropyl, 2-fluoropropyl, 3-fluoropropyl, 3,3-difluoropropyl, 3,3,3-trifluoropropyl, heptafluoropropyl, 1,1,1-trifluoroprop-2-yl, 3-chloropropyl, 4-chlorobutyl and the like.

The term “cycloalkyl” as used herein (and in the cycloalkyl moieties of other groups comprising a cycloalkyl group, e.g. cycloalkoxy and cycloalkylalkyl) denotes in each case a mono- or bicyclic cycloaliphatic radical having usually from 3 to 10 carbon atoms (“C₃-C₁₀-cycloalkyl”), preferably 3 to 8 carbon atoms (“C₃-C₈-cycloalkyl”) or in particular 3 to 6 carbon atoms (“C₃-C₆-cycloalkyl”). Examples of monocyclic radicals having 3 to 6 carbon atoms comprise cyclopropyl, cyclobutyl, cyclopentyl and cyclohexyl. Examples of monocyclic radicals having 3 to 8 carbon atoms comprise cyclopropyl, cyclobutyl, cyclopentyl, cyclohexyl, cycloheptyl and cyclooctyl. Examples of bicyclic radicals having 7 or 8 carbon atoms comprise bicyclo[2.1.1]hexyl, bicyclo[2.2.1]heptyl, bicyclo[3.1.1]heptyl, bicyclo[2.2.1]heptyl, bicyclo[2.2.2]octyl and bicyclo[3.2.1]octyl.

The term “cycloalkylene” (or cycloalkanediyl) as used herein in each case denotes an cycloalkyl radical as defined above, wherein one hydrogen atom at any position of the carbon backbone is replaced by one further binding site, thus forming a bivalent moiety.

The term “halocycloalkyl” as used herein (and in the halocycloalkyl moieties of other groups comprising an halocycloalkyl group, e.g. halocycloalkylmethyl) denotes in each case a mono- or bicyclic cycloaliphatic radical having usually from 3 to 10 carbon atoms, preferably 3 to 8 carbon atoms or in particular 3 to 6 carbon atoms, wherein at least one, e.g. 1, 2, 3, 4 or 5 of the hydrogen atoms are replaced by halogen, in particular by fluorine or chlorine. Examples are 1- and 2-fluorocyclopropyl, 1,2-, 2,2- and 2,3-difluorocyclopropyl, 1,2,2-trifluorocyclopropyl, 2,2,3,3-tetrafluorocyclpropyl, 1- and 2-chlorocyclopropyl, 1,2-, 2,2- and 2,3-dichlorocyclopropyl, 1,2,2-trichlorocyclopropyl, 2,2,3,3-tetrachlorocyclpropyl, 1-, 2- and 3-fluorocyclopentyl, 1,2-, 2,2-, 2,3-, 3,3-, 3,4-, 2,5-difluorocyclopentyl, 1-, 2- and 3-chlorocyclopentyl, 1,2-, 2,2-, 2,3-, 3,3-, 3,4-, 2,5-dichlorocyclopentyl and the like.

The term “cycloalkyl-alkyl” used herein denotes a cycloalkyl group, as defined above, which is bound to the remainder of the molecule via an alkylene group. The term “C₃-C₈-cycloalkyl-C₁-C₄-alkyl” refers to a C₃-C₈-cycloalkyl group as defined above which is bound to the remainder of the molecule via a C₁-C₄-alkyl group, as defined above. Examples are cyclopropylmethyl, cyclopropylethyl, cyclopropylpropyl, cyclobutylmethyl, cyclobutylethyl, cyclobutylpropyl, cyclopentylmethyl, cyclopentylethyl, cyclopentylpropyl, cyclohexylmethyl, cyclohexylethyl, cyclohexylpropyl, and the like.

The term “alkenyl” as used herein denotes in each case a monounsaturated straight-chain or branched hydrocarbon radical having usually 2 to 10 (“C₂-C₁₀-alkenyl”), preferably 2 to 6 carbon atoms (“C₂-C₆-alkenyl”), in particular 2 to 4 carbon atoms (“C₂-C₄-alkenyl”), and a double bond in any position, for example C₂-C₄-alkenyl, such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl or 2-methyl-2-propenyl; C₂-C₆-alkenyl, such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl, 1-ethyl-2-methyl-2-propenyl and the like, or C₂-C₁₀-alkenyl, such as the radicals mentioned for C₂-C₆-alkenyl and additionally 1-heptenyl, 2-heptenyl, 3-heptenyl, 1-octenyl, 2-octenyl, 3-octenyl, 4-octenyl, 1-nonenyl, 2-nonenyl, 3-nonenyl, 4-nonenyl, 1-decenyl, 2-decenyl, 3-decenyl, 4-decenyl, 5-decenyl and the positional isomers thereof.

The term “alkenylene” (or alkenediyl) as used herein in each case denotes an alkenyl radical as defined above, wherein one hydrogen atom at any position of the carbon backbone is replaced by one further binding site, thus forming a bivalent moiety.

The term “haloalkenyl” as used herein, which may also be expressed as “alkenyl which may be substituted by halogen”, and the haloalkenyl moieties in haloalkenyloxy, haloalkenylcarbonyl and the like refers to unsaturated straight-chain or branched hydrocarbon radicals having 2 to 10 (“C₂-C₁₀-haloalkenyl”) or 2 to 6 (“C₂-C₆-haloalkenyl”) or 2 to 4 (“C₂-C₄-haloalkenyl”) carbon atoms and a double bond in any position, where some or all of the hydrogen atoms in these groups are replaced by halogen atoms as mentioned above, in particular fluorine, chlorine and bromine, for example chlorovinyl, chloroallyl and the like.

The term “alkynyl” as used herein denotes unsaturated straight-chain or branched hydrocarbon radicals having usually 2 to 10 (“C₂-C₁₀-alkynyl”), frequently 2 to 6 (“C₂-C₆-alkynyl”), preferably 2 to 4 carbon atoms (“C₂-C₄-alkynyl”) and one or two triple bonds in any position, for example C₂-C₄-alkynyl, such as ethynyl, 1-propynyl, 2-propynyl, 1-butynyl, 2-butynyl, 3-butynyl, 1-methyl-2-propynyl and the like, C₂-C₆-alkynyl, such as ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl, 1-ethyl-1-methyl-2-propynyl and the like.

The term “alkynylene” (or alkynediyl) as used herein in each case denotes an alkynyl radical as defined above, wherein one hydrogen atom at any position of the carbon backbone is replaced by one further binding site, thus forming a bivalent moiety.

The term “haloalkynyl” as used herein, which is also expressed as “alkynyl which may be substituted by halogen”, refers to unsaturated straight-chain or branched hydrocarbon radicals having usually 3 to 10 carbon atoms (“C₂-C₁₀-haloalkynyl”), frequently 2 to 6 (“C₂-C₆-haloalkynyl”), preferably 2 to 4 carbon atoms (“C₂-C₄-haloalkynyl”), and one or two triple bonds in any position (as mentioned above), where some or all of the hydrogen atoms in these groups are replaced by halogen atoms as mentioned above, in particular fluorine, chlorine and bromine.

The term “alkoxy” as used herein denotes in each case a straight-chain or branched alkyl group usually having from 1 to 10 carbon atoms (“C₁-C₁₀-alkoxy”), frequently from 1 to 6 carbon atoms (“C₁-C₆-alkoxy”), preferably 1 to 4 carbon atoms (“C₁-C₄-alkoxy”), which is bound to the remainder of the molecule via an oxygen atom. C₁-C₂-Alkoxy is methoxy or ethoxy. C₁-C₄-Alkoxy is additionally, for example, n-propoxy, 1-methylethoxy (isopropoxy), butoxy, 1-methylpropoxy (sec-butoxy), 2-methylpropoxy (isobutoxy) or 1,1-dimethylethoxy (tert-butoxy). C₁-C₆-Alkoxy is additionally, for example, pentoxy, 1-methylbutoxy, 2-methylbutoxy, 3-methylbutoxy, 1,1-dimethylpropoxy, 1,2-dimethylpropoxy, 2,2-dimethylpropoxy, 1-ethylpropoxy, hexoxy, 1-methylpentoxy, 2-methylpentoxy, 3-methylpentoxy, 4-methylpentoxy, 1,1-dimethylbutoxy, 1,2-dimethylbutoxy, 1,3-dimethylbutoxy, 2,2-dimethylbutoxy, 2,3-dimethylbutoxy, 3,3-dimethylbutoxy, 1-ethylbutoxy, 2-ethylbutoxy, 1,1,2-trimethylpropoxy, 1,2,2-trimethylpropoxy, 1-ethyl-1-methylpropoxy or 1-ethyl-2-methylpropoxy. C₁-C₈-Alkoxy is additionally, for example, heptyloxy, octyloxy, 2-ethylhexyloxy and positional isomers thereof. C₁-C₁₀-Alkoxy is additionally, for example, nonyloxy, decyloxy and positional isomers thereof.

The term “haloalkoxy” as used herein denotes in each case a straight-chain or branched alkoxy group, as defined above, having from 1 to 10 carbon atoms (“C₁-C₁₀-haloalkoxy”), frequently from 1 to 6 carbon atoms (“C₁-C₆-haloalkoxy”), preferably 1 to 4 carbon atoms (“C₁-C₄-haloalkoxy”), more preferably 1 to 3 carbon atoms (“C₁-C₃-haloalkoxy”), wherein the hydrogen atoms of this group are partially or totally replaced with halogen atoms, in particular fluorine atoms. C₁-C₂-Haloalkoxy is, for example, OCH₂F, OCHF₂, OCF₃, OCH₂Cl, OCHCl₂, OCCl₃, chlorofluoromethoxy, dichlorofluoromethoxy, chlorodifluoromethoxy, 2-fluoroethoxy, 2-chloroethoxy, 2-bromoethoxy, 2-iodoethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-trichloroethoxy or OC₂F₅. C₁-C₄-Haloalkoxy is additionally, for example, 2-fluoropropoxy, 3-fluoropropoxy, 2,2-difluoropropoxy, 2,3-difluoropropoxy, 2-chloropropoxy, 3-chloropropoxy, 2,3-dichloropropoxy, 2-bromopropoxy, 3-bromopropoxy, 3,3,3-trifluoropropoxy, 3,3,3-trichloropropoxy, OCH₂—C₂F₅, OCF₂—C₂F₅, 1-(CH₂F)-2-fluoroethoxy, 1-(CH₂Cl)-2-chloroethoxy, 1-(CH₂Br)-2-bromoethoxy, 4-fluorobutoxy, 4-chlorobutoxy, 4-bromobutoxy or nonafluorobutoxy. C₁-C₆-Haloalkoxy is additionally, for example, 5-fluoropentoxy, 5-chloropentoxy, 5-bromopentoxy, 5-iodopentoxy, undecafluoropentoxy, 6-fluorohexoxy, 6-chlorohexoxy, 6-bromohexoxy, 6-iodohexoxy or dodecafluorohexoxy.

The term “alkoxyalkyl” as used herein denotes in each case alkyl usually comprising 1 to 6 carbon atoms, preferably 1 to 4 carbon atoms, wherein 1 carbon atom carries an alkoxy radical usually comprising 1 to 10, frequently 1 to 6, in particular 1 to 4, carbon atoms as defined above. “C₁-C₆-Alkoxy-C₁-C₆-alkyl” is a C₁-C₆-alkyl group, as defined above, in which one hydrogen atom is replaced by a C₁-C₆-alkoxy group, as defined above. Examples are CH₂OCH₃, CH₂—OC₂H₅, n-propoxymethyl, CH₂—OCH(CH₃)₂, n-butoxymethyl, (1-methylpropoxy)-methyl, (2-methylpropoxy)methyl, CH₂—OC(CH₃)₃, 2-(methoxy)ethyl, 2-(ethoxy)ethyl, 2-(n-propoxy)-ethyl, 2-(1-methylethoxy)-ethyl, 2-(n-butoxy)ethyl, 2-(1-methylpropoxy)-ethyl, 2-(2-methylpropoxy)-ethyl, 2-(1,1-dimethylethoxy)-ethyl, 2-(methoxy)-propyl, 2-(ethoxy)-propyl, 2-(n-propoxy)-propyl, 2-(1-methylethoxy)-propyl, 2-(n-butoxy)-propyl, 2-(1-methylpropoxy)-propyl, 2-(2-methylpropoxy)propyl, 2-(1,1-dimethylethoxy)-propyl, 3-(methoxy)-propyl, 3-(ethoxy)-propyl, 3-(n-propoxy)propyl, 3-(1-methylethoxy)-propyl, 3-(n-butoxy)-propyl, 3-(1-methylpropoxy)-propyl, 3-(2-methylpropoxy)-propyl, 3-(1,1-dimethylethoxy)-propyl, 2-(methoxy)-butyl, 2-(ethoxy)-butyl, 2-(n-propoxy)-butyl, 2-(1-methylethoxy)-butyl, 2-(n-butoxy)-butyl, 2-(1-methylpropoxy)-butyl, 2-(2-methyl-propoxy)-butyl, 2-(1,1-dimethylethoxy)-butyl, 3-(methoxy)-butyl, 3-(ethoxy)-butyl, 3-(n-propoxy)-butyl, 3-(1-methylethoxy)-butyl, 3-(n-butoxy)-butyl, 3-(1-methylpropoxy)-butyl, 3-(2-methylpropoxy)-butyl, 3-(1,1-dimethylethoxy)-butyl, 4-(methoxy)-butyl, 4-(ethoxy)-butyl, 4-(n-propoxy)-butyl, 4-(1-methylethoxy)-butyl, 4-(n-butoxy)-butyl, 4-(1-methylpropoxy)-butyl, 4-(2-methylpropoxy)-butyl, 4-(1,1-dimethylethoxy)-butyl and the like.

The term “haloalkoxy-alkyl” as used herein denotes in each case alkyl as defined above, usually comprising 1 to 6 carbon atoms, preferably 1 to 4 carbon atoms, wherein 1 carbon atom carries an haloalkoxy radical as defined above, usually comprising 1 to 10, frequently 1 to 6, in particular 1 to 4, carbon atoms as defined above. Examples are fluoromethoxymethyl, difluoromethoxymethyl, trifluoromethoxymethyl, 1-fluoroethoxymethyl, 2-fluoroethoxymethyl, 1,1-difluoroethoxymethyl, 1,2-difluoroethoxymethyl, 2,2-difluoroethoxymethyl, 1,1,2-trifluoroethoxymethyl, 1,2,2-trifluoroethoxymethyl, 2,2,2-trifluoroethoxymethyl, pentafluoroethoxymethyl, 1-fluoroethoxy-1-ethyl, 2-fluoroethoxy-1-ethyl, 1,1-difluoroethoxy-1-ethyl, 1,2-difluoroethoxy-1-ethyl, 2,2-difluoroethoxy-1-ethyl, 1,1,2-trifluoroethoxy-1-ethyl, 1,2,2-trifluoroethoxy-1-ethyl, 2,2,2-trifluoroethoxy-1-ethyl, pentafluoroethoxy-1-ethyl, 1-fluoroethoxy-2-ethyl, 2-fluoroethoxy-2-ethyl, 1,1-difluoroethoxy-2-ethyl, 1,2-difluoroethoxy-2-ethyl, 2,2-difluoroethoxy-2-ethyl, 1,1,2-trifluoroethoxy-2-ethyl, 1,2,2-trifluoroethoxy-2-ethyl, 2,2,2-trifluoroethoxy-2-ethyl, pentafluoroethoxy-2-ethyl, and the like.

The term “alkylthio” (also alkylsulfanyl or alkyl-S—)” as used herein denotes in each case a straight-chain or branched saturated alkyl group as defined above, usually comprising 1 to 10 carbon atoms (“C₁-C₁₀-alkylthio”), frequently comprising 1 to 6 carbon atoms (“C₁-C₆-alkylthio”), preferably 1 to 4 carbon atoms (“C₁-C₄-alkylthio”), which is attached via a sulfur atom at any position in the alkyl group. C₁-C₂-Alkylthio is methylthio or ethylthio. C₁-C₄-Alkylthio is additionally, for example, n-propylthio, 1-methylethylthio (isopropylthio), butylthio, 1-methylpropylthio (sec-butylthio), 2-methylpropylthio (isobutylthio) or 1,1-dimethylethylthio (tert-butylthio). C₁-C₆-Alkylthio is additionally, for example, pentylthio, 1-methylbutylthio, 2-methylbutylthio, 3-methylbutylthio, 1,1-dimethylpropylthio, 1,2-dimethylpropylthio, 2,2-dimethylpropylthio, 1-ethylpropylthio, hexylthio, 1-methylpentylthio, 2-methylpentylthio, 3-methylpentylthio, 4-methylpentylthio, 1,1-dimethylbutylthio, 1,2-dimethylbutylthio, 1,3-dimethylbutylthio, 2,2-dimethylbutylthio, 2,3-dimethylbutylthio, 3,3-dimethylbutylthio, 1-ethylbutylthio, 2-ethylbutylthio, 1,1,2-trimethylpropylthio, 1,2,2-trimethylpropylthio, 1-ethyl-1-methylpropylthio or 1-ethyl-2-methylpropylthio. C₁-C₈-Alkylthio is additionally, for example, heptylthio, octylthio, 2-ethylhexylthio and positional isomers thereof. C₁-C₁₀-Alkylthio is additionally, for example, nonylthio, decylthio and positional isomers thereof.

The term “haloalkylthio” as used herein refers to an alkylthio group as defined above wherein the hydrogen atoms are partially or fully substituted by fluorine, chlorine, bromine and/or iodine. C₁-C₂-Haloalkylthio is, for example, SCH₂F, SCHF₂, SCF₃, SCH₂Cl, SCHCl₂, SCCl₃, chlorofluoromethylthio, dichlorofluoromethylthio, chlorodifluoromethylthio, 2-fluoroethylthio, 2-chloroethylthio, 2-bromoethylthio, 2-iodoethylthio, 2,2-difluoroethylthio, 2,2,2-trifluoroethylthio, 2-chloro-2-fluoroethylthio, 2-chloro-2,2-difluoroethylthio, 2,2-dichloro-2-fluoroethylthio, 2,2,2-trichloroethylthio or SC₂F₅. C₁-C₄-Haloalkylthio is additionally, for example, 2-fluoropropylthio, 3-fluoropropylthio, 2,2-difluoropropylthio, 2,3-difluoropropylthio, 2-chloropropylthio, 3-chloropropylthio, 2,3-dichloropropylthio, 2-bromopropylthio, 3-bromopropylthio, 3,3,3-trifluoropropylthio, 3,3,3-trichloropropylthio, SCH₂—C₂F₅, SCF₂—C₂F₅, 1-(CH₂F)-2-fluoroethylthio, 1-(CH₂Cl)-2-chloroethylthio, 1-(CH₂Br)-2-bromoethylthio, 4-fluorobutylthio, 4-chlorobutylthio, 4-bromobutylthio or nonafluorobutylthio. C₁-C₆-Haloalkylthio is additionally, for example, 5-fluoropentylthio, 5-chloropentylthio, 5-brompentylthio, 5-iodopentylthio, undecafluoropentylthio, 6-fluorohexylthio, 6-chlorohexylthio, 6-bromohexylthio, 6-iodohexylthio or dodecafluorohexylthio.

The terms “alkylsulfinyl” and “S(O)ₙ-alkyl” (wherein n is 1) are equivalent and, as used herein, denote an alkyl group, as defined above, attached via a sulfinyl [S(O)] group. For example, the term “C₁-C₂-alkylsulfinyl” refers to a C₁-C₂-alkyl group, as defined above, attached via a sulfinyl [S(O)] group. The term “C₁-C₄-alkylsulfinyl” refers to a C₁-C₄-alkyl group, as defined above, attached via a sulfinyl [S(O)] group. The term “C₁-C₆-alkylsulfinyl” refers to a C₁-C₆-alkyl group, as defined above, attached via a sulfinyl [S(O)] group. C₁-C₂-alkylsulfinyl is methylsulfinyl or ethylsulfinyl. C₁-C₄-alkylsulfinyl is additionally, for example, n-propylsulfinyl, 1-methylethylsulfinyl (isopropylsulfinyl), butylsulfinyl, 1-methylpropylsulfinyl (sec-butylsulfinyl), 2-methylpropylsulfinyl (isobutylsulfinyl) or 1,1-dimethylethylsulfinyl (tert-butylsulfinyl). C₁-C₆-alkylsulfinyl is additionally, for example, pentylsulfinyl, 1-methylbutylsulfinyl, 2-methylbutylsulfinyl, 3-methylbutylsulfinyl, 1,1-dimethylpropylsulfinyl, 1,2-dimethylpropylsulfinyl, 2,2-dimethylpropylsulfinyl, 1-ethylpropylsulfinyl, hexylsulfinyl, 1-methylpentylsulfinyl, 2-methylpentylsulfinyl, 3-methylpentylsulfinyl, 4-methylpentylsulfinyl, 1,1-dimethylbutylsulfinyl, 1,2-dimethylbutylsulfinyl, 1,3-dimethylbutylsulfinyl, 2,2-dimethylbutylsulfinyl, 2,3-dimethylbutylsulfinyl, 3,3-dimethylbutylsulfinyl, 1-ethylbutylsulfinyl, 2-ethylbutylsulfinyl, 1,1,2-trimethylpropylsulfinyl, 1,2,2-trimethylpropylsulfinyl, 1-ethyl-1-methylpropylsulfinyl or 1-ethyl-2-methylpropylsulfinyl.

The terms “alkylsulfonyl” and “S(O)ₙ-alkyl” (wherein n is 2) are equivalent and, as used herein, denote an alkyl group, as defined above, attached via a sulfonyl [S(O)₂] group. The term “C₁-C₂-alkylsulfonyl” refers to a C₁-C₂-alkyl group, as defined above, attached via a sulfonyl [S(O)₂] group. The term “C₁-C₄-alkylsulfonyl” refers to a C₁-C₄-alkyl group, as defined above, attached via a sulfonyl [S(O)₂] group. The term “C₁-C₆-alkylsulfonyl” refers to a C₁-C₆-alkyl group, as defined above, attached via a sulfonyl [S(O)₂] group. C₁-C₂-alkylsulfonyl is methylsulfonyl or ethylsulfonyl. C₁-C₄-alkylsulfonyl is additionally, for example, n-propylsulfonyl, 1-methylethylsulfonyl (isopropylsulfonyl), butylsulfonyl, 1-methylpropylsulfonyl (sec-butylsulfonyl), 2-methylpropylsulfonyl (isobutylsulfonyl) or 1,1-dimethylethylsulfonyl (tert-butylsulfonyl). C₁-C₆-alkylsulfonyl is additionally, for example, pentylsulfonyl, 1-methylbutylsulfonyl, 2-methylbutylsulfonyl, 3-methylbutylsulfonyl, 1,1-dimethylpropylsulfonyl, 1,2-dimethylpropylsulfonyl, 2,2-dimethylpropylsulfonyl, 1-ethylpropylsulfonyl, hexylsulfonyl, 1-methylpentylsulfonyl, 2-methylpentylsulfonyl, 3-methylpentylsulfonyl, 4-methylpentylsulfonyl, 1,1-dimethylbutylsulfonyl, 1,2-dimethylbutylsulfonyl, 1,3-dimethylbutylsulfonyl, 2,2-dimethylbutylsulfonyl, 2,3-dimethylbutylsulfonyl, 3,3-dimethylbutylsulfonyl, 1-ethylbutylsulfonyl, 2-ethylbutylsulfonyl, 1,1,2-trimethylpropylsulfonyl, 1,2,2-trimethylpropylsulfonyl, 1-ethyl-1-methylpropylsulfonyl or 1-ethyl-2-methylpropylsulfonyl.

The term “alkylamino” as used herein denotes in each case a group —NHR, wherein R is a straight-chain or branched alkyl group usually having from 1 to 6 carbon atoms (“C₁-C₆-alkylamino”), preferably 1 to 4 carbon atoms (“C₁-C₄-alkylamino”). Examples of C₁-C₆-alkylamino are methylamino, ethylamino, n-propylamino, isopropylamino, n-butylamino, 2-butylamino, isobutylamino, tert-butylamino, and the like.

The term “dialkylamino” as used herein denotes in each case a group-NRR′, wherein R and R′, independently of each other, are a straight-chain or branched alkyl group each usually having from 1 to 6 carbon atoms (“di-(C₁-C₆-alkyl)-amino”), preferably 1 to 4 carbon atoms (“di-(C₁-C₄-alkyl)-amino”). Examples of a di-(C₁-C₆-alkyl)-amino group are dimethylamino, diethylamino, dipropylamino, dibutylamino, methyl-ethyl-amino, methyl-propyl-amino, methyl-isopropylamino, methyl-butyl-amino, methyl-isobutyl-amino, ethyl-propyl-amino, ethyl-isopropylamino, ethyl-butyl-amino, ethyl-isobutyl-amino, and the like.

The term “cycloalkylamino” as used herein denotes in each case a group —NHR, wherein R is a cycloalkyl group usually having from 3 to 8 carbon atoms (“C₃-C₈-cycloalkylamino”), preferably 3 to 6 carbon atoms (“C₃-C₆-cycloalkylamino”). Examples of C₃-C₈-cycloalkylamino are cyclopropylamino, cyclobutylamino, cyclopentylamino, cyclohexylamino, and the like.

The term “alkylaminosulfonyl” as used herein denotes in each case a straight-chain or branched alkylamino group as defined above, which is bound to the remainder of the molecule via a sulfonyl [S(O)₂] group. Examples of an alkylaminosulfonyl group are methylaminosulfonyl, ethylaminosulfonyl, n-propylaminosulfonyl, isopropylaminosulfonyl, n-butylaminosulfonyl, 2-butylaminosulfonyl, iso-butylaminosulfonyl, tert-butylaminosulfonyl, and the like.

The term “dialkylaminosulfonyl” as used herein denotes in each case a straight-chain or branched alkylamino group as defined above, which is bound to the remainder of the molecule via a sulfonyl [S(O)₂] group. Examples of an dialkylaminosulfonyl group are dimethylaminosulfonyl, diethylaminosulfonyl, dipropylaminosulfonyl, dibutylaminosulfonyl, methyl-ethylaminosulfonyl, methyl-propyl-aminosulfonyl, methyl-isopropylaminosulfonyl, methyl-butyl-aminosulfonyl, methyl-isobutyl-aminosulfonyl, ethyl-propyl-aminosulfonyl, ethyl-isopropylaminosulfonyl, ethyl-butyl-aminosulfonyl, ethyl-isobutyl-aminosulfonyl, and the like.

The suffix “-carbonyl” in a group denotes in each case that the group is bound to the remainder of the molecule via a carbonyl C═O group. This is the case e.g. in alkylcarbonyl, haloalkylcarbonyl, aminocarbonyl, alkylaminocarbonyl, dialkylaminocarbonyl, alkoxycarbonyl, haloalkoxycarbonyl.

The term “aryl” as used herein refers to a mono-, bi- or tricyclic aromatic hydrocarbon radical such as phenyl or naphthyl, in particular phenyl.

The term “het(ero)aryl” as used herein refers to a mono-, bi- or tricyclic heteroaromatic hydrocarbon radical, preferably to a monocyclic heteroaromatic radical, such as pyridyl, pyrimidyl and the like.

A saturated, partially unsaturated or unsaturated 3- to 8-membered ring system which contains 1 to 4 heteroatoms selected from oxygen, nitrogen, sulfur, is a ring system wherein two oxygen atoms must not be in adjacent positions and wherein at least 1 carbon atom must be in the ring system e.g. thiophene, furan, pyrrole, thiazole, oxazole, imidazole, isothiazole, isoxazole, pyrazole, 1,3,4-oxadiazole, 1,3,4-thiadiazole, 1,3,4-triazole, 1,2,4-oxadiazole, 1,2,4-thiadiazole, 1,2,4-triazole, 1,2,3-triazole, 1,2,3,4-tetrazole, benzo[b]thiophene, benzo[b]furan, indole, benzo[c]thiophene, benzo[c]furan, isoindole, benzoxazole, benzthiazole, benzimidazole, benzisoxazole, benzisothiazole, benzopyrazole, benzothiadiazole, benztriazole, dibenzofuran, dibenzothiophene, carbazole, pyridine, pyrazine, pyrimidine, pyridazine, 1,3,5-triazine, 1,2,4-triazine, 1,2,4,5-tetrazine, quinoline, isoquinoline, quinoxaline, quinazoline, cinnoline, 1,8-naphthyridine, 1,5-naphthyridine, 1,6-naphthyridine, 1,7-naphthyridine, phthalazine, pyridopyrimidine, purine, pteridine, 4H-quinolizine, piperidine, pyrrolidine, oxazoline, tetrahydrofuran, tetrahydropyran, isoxazolidine or thiazolidine, oxirane or oxetane.

A saturated, partially unsaturated or unsaturated 3- to 8-membered ring system which contains 1 to 4 heteroatoms selected from oxygen, nitrogen, sulfur also is e.g. a saturated, partially unsaturated or unsaturated 5- or 6-membered heterocycle which contains 1 to 4 heteroatoms selected from oxygen, nitrogen and sulfur, such as pyridine, pyrimidine, (1,2,4)-oxadiazole, (1,3,4)-oxadiazole, pyrrole, furan, thiophene, oxazole, thiazole, imidazole, pyrazole, isoxazole, 1,2,4-triazole, tetrazole, pyrazine, pyridazine, oxazoline, thiazoline, tetrahydrofuran, tetrahydropyran, morpholine, piperidine, piperazine, pyrroline, pyrrolidine, oxazolidine, thiazolidine; or

a saturated, partially unsaturated or unsaturated 5- or 6-membered heterocycle which contains 1 nitrogen atom and 0 to 2 further heteroatoms selected from oxygen, nitrogen and sulfur, preferably from oxygen and nitrogen, such as piperidine, piperazin and morpholine.

Preferably, this ring system is a saturated, partially unsaturated or unsaturated 3- to 6-membered ring system which contains 1 to 4 heteroatoms selected from oxygen, nitrogen, sulfur, wherein two oxygen atoms must not be in adjacent positions and wherein at least 1 carbon atom must be in the ring system.

Most preferably, this ring system is a radical of pyridine, pyrimidine, (1,2,4)-oxadiazole, 1,3,4-oxadiazole, pyrrole, furan, thiophene, oxazole, thiazole, imidazole, pyrazole, isoxazole, 1,2,4-triazole, tetrazole, pyrazine, pyridazine, oxazoline, thiazoline, tetrahydrofuran, tetrahydropyran, morpholine, piperidine, piperazine, pyrroline, pyrrolidine, oxazolidine, thiazolidine, oxirane or oxetane.

Preparation of the compounds of formula I can be accomplished according to standard methods of organic chemistry, e.g. by the methods or working examples described in WO 2007/006670, PCT/EP2012/065650 and PCT/EP2012/065651, without being limited to the routes given therein.

The preparation of the compounds of formula I above may lead to them being obtained as isomer mixtures. If desired, these can be resolved by the methods customary for this purpose, such as crystallization or chromatography, also on optically active adsorbate, to give the pure isomers.

Agronomically acceptable salts of the compounds I can be formed in a customary manner, e.g. by reaction with an acid of the anion in question.

### Preferences

The remarks made below as to preferred embodiments of the variables (substituents) of the compounds of formulae (I) are valid on their own as well as preferably in combination with each other, as well as in combination with the stereoisomers, tautomers, N-oxides or salts thereof, and, where applicable, as well as concerning the uses and methods according to the invention and the compositions according to the invention.

Preferred compounds according to the invention are compounds of formulae (I) or a stereoisomer, N-oxide or salt thereof, wherein the salt is an agriculturally or veterinarily acceptable salt. The compounds I of formula (I) and their examples include their tautomers, racemic mixtures, individual pure enantiomers and diastereomers and their optically active mixtures.

Preferred are methods and uses of compounds of formula (I), wherein the compound of formula I is a compound of formula IA:

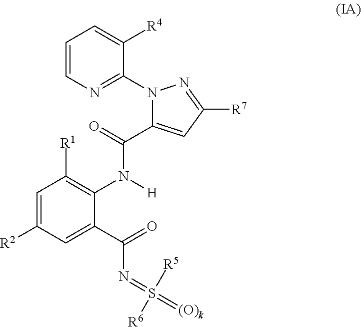

wherein
R⁴ is halogen, and
wherein the variables R¹, R², R⁷, R⁵, R⁶ and k are as defined herein.

Preferred are methods and uses of compounds of formula (I), in which the compound of formula I is a compound of formula IB:

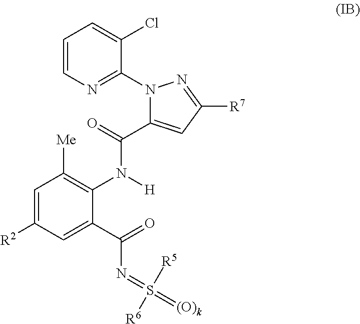

wherein
R² is selected from the group consisting of bromo, chloro, cyano;
R⁷ is selected from the group consisting of bromo, chloro, trifluoromethyl. OCHF₂, and
wherein the variables R², R⁷, R⁵, R⁶ and k are as defined herein.

Preferred are methods and uses of compounds of formula (I), in which the compound of formula I is a compound of formula IC:

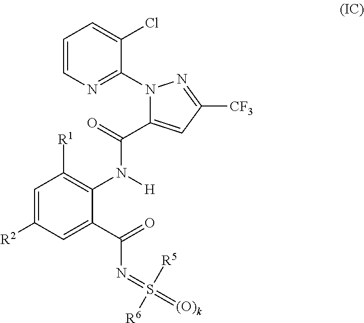

wherein
R¹ is selected from the group consisting of halogen and halomethyl;
R² is selected from the group consisting of bromo, chloro and cyano, and
wherein the variables R⁵, R⁶ and k are as defined herein.

Preferred are methods and uses of compounds of formula (I), in which the compound of formula I is a compound of formula ID:

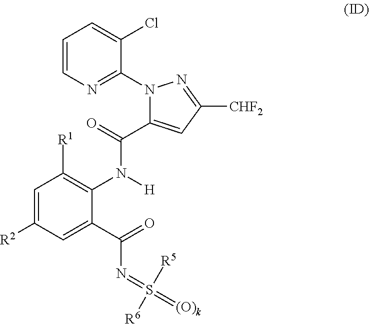

wherein
R¹ is selected from the group consisting of halogen, methyl and halomethyl;
R² is selected from the group consisting of bromo, chloro and cyano, and
wherein the variables R⁵, R⁶ and k are as defined herein.

Preferred are methods and uses of compounds of formula (I), in which R⁵, R⁶ are selected independently of one another from the group consisting of hydrogen, C₁-C₁₀-alkyl, C₃-C₈-cycloalkyl, wherein the aforementioned aliphatic and cycloaliphatic radicals may be substituted with 1 to 10 substituents R^{e}; or
R⁵ and R⁶ together represent a C₂-C₇-alkylene chain forming together with the sulfur atom to which they are attached a 3-, 4-, 5-, 6-, 7- or 8-membered saturated, partially unsaturated or fully unsaturated ring, wherein 1 to 4 of the CH₂ groups in the C₂-C₇-alkylene chain may be replaced by 1 to 4 groups independently selected from the group consisting of C═O, C═S, O, S, N, NO, SO, SO₂ and NH, and wherein the carbon and/or nitrogen atoms in the C₂-C₇-alkylene chain may be substituted with 1 to 5 substituents independently selected from the group consisting of halogen, cyano, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkynyl and C₂-C₆-haloalkynyl; said substituents being identical or different from one another if more than one substituent is present.

Preferred are methods and uses of compounds of formula (I), in which R⁵, R⁶ are selected independently of one another from the group consisting of hydrogen, C₁-C₁₀-alkyl, C₃-C₈-cycloalkyl, wherein the aforementioned aliphatic and cycloaliphatic radicals may be substituted with 1 to 10 substituents R^{e}.

Preferred are methods and uses of compounds of formula (I), in which R⁷ is selected from the group consisting of bromo, difluoromethyl, trifluoromethyl, cyano, OCHF₂, OCH₂F and OCH₂CF₃,

Preferred are methods and uses of compounds of formula (I), in which R⁷ is selected from the group consisting of bromo, difluoromethyl, trifluoromethyl and OCHF₂.

Preferred are methods and uses of compounds of formula (I), in which R^{e} is independently selected from the group consisting of halogen, cyano, —OH, —SH, —SCN, C₁-C₆-alkyl, C₂-C₆-alkenyl, C₂-C₆-alkinyl, C₃-C₈-cycloalkyl, wherein one or more CH₂ groups of the aforementioned radicals may be replaced by a C═O group, and/or the aliphatic and cycloaliphatic moieties of the aforementioned radicals may be unsubstituted, partially or fully halogenated and/or may carry 1 or 2 radicals selected from C₁-C₆-alkoxy, C₁-C₆-haloalkoxy, C₁-C₆-alkylthio, C₁-C₆-alkylsulfinyl, C₁-C₆-alkylsulfonyl, C₁-C₆-haloalkylthio, —OR^{a}, —NR^{c}R^{d}, —S(O)ₙR^{a}, —S(O)ₙNR^{c}R^{d}, —C(═O)R^{a}, —C(═O)NR^{c}R^{d}, —C(═O)OR^{b}, —C(═S)R^{a}, —C(═S)NR^{c}R^{d}, —C(═S)OR^{b}, —C(═S)SR^{b}, —C(═NR^{c})R^{b}, —C(═NR^{c})NR^{c}R^{d}, phenyl, benzyl, pyridyl and phenoxy, wherein the last four radicals may be unsubstituted, partially or fully halogenated and/or carry 1, 2 or 3 substituents selected from C₁-C₆-alkyl, C₁-C₆-haloalkyl, C₁-C₆-alkoxy and C₁-C₆-haloalkoxy.

Preferred are methods and uses of compounds of formula (I), in which R^{e} is independently selected from the group consisting of halogen, cyano, —OH, —SH, —SCN, C₁-C₆-alkyl, C₂-C₆-alkenyl, C₂-C₆-alkinyl, C₃-C₈-cycloalkyl, wherein one or more CH₂ groups of the aforementioned radicals may be replaced by a C═O group, and/or the aliphatic and cycloaliphatic moieties of the aforementioned radicals may be unsubstituted, partially or fully halogenated.

Preferred are methods and uses of compounds of formula (I) as described herein, in which in the compound of formula I

R⁵ and R⁶ are selected from methyl, ethyl, isopropyl, n-propyl, n-butyl, isobutyl, tert-butyl, cyclopropyl, cyclopropylmethyl.

Preferred are methods and uses of compounds of formula (I) as described herein, in which in the compound of formula I

R⁵ and R⁶ are identical.

In a particularly preferred embodiment, the methods and uses according to the invention comprise at least one compound of formula (IA)

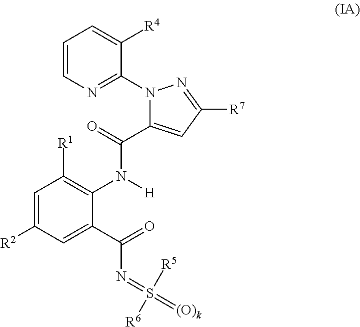

wherein
R⁴ is Cl,
R¹ is selected from the group consisting of Cl, Br, and methyl;
R² is selected from the group consisting of bromo and chloro;
R⁵, R⁶ are selected independently of one another from the group consisting of methyl, ethyl, n-propyl, isopropyl, n-butyl, isobutyl, tert-butyl.
R⁷ is selected from the group consisting of difluoromethyl, trifluoromethyl.

Examples of especially preferred anthranilamide compounds I of the present invention are of formula (IA-1)

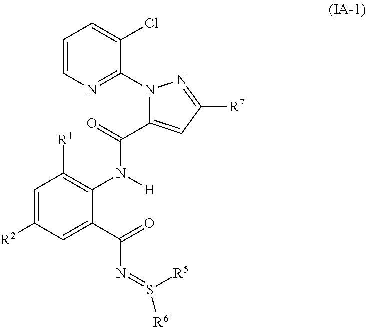

wherein R¹, R², R⁷, R⁵, R⁶ are as defined herein.

Examples of preferred compounds of formula I in the methods and uses according to the invention are compiled in tables 1 to 60 below. Moreover, the meanings mentioned below for the individual variables in the tables are per se, independently of the combination in which they are mentioned, a particularly preferred embodiment of the substituents in question.

Table 1 Compounds of the formula (IA-1) in which R¹ is F, R² is Cl, R⁷ is CF₃ and the combination of R⁵ and R⁶ for a compound corresponds in each case to one row of Table A;

Table 2 Compounds of the formula (IA-1) in which R¹ is Br, R² is Cl, R⁷ is CF₃ and the combination of R⁵ and R⁶ for a compound corresponds in each case to one row of Table A;

Table 3 Compounds of the formula (IA-1) in which R¹ is Cl, R² is Cl, R⁷ is CF₃ and the combination of R⁵ and R⁶ for a compound corresponds in each case to one row of Table A;

Table 4 Compounds of the formula (IA-1) in which R¹ is methyl, R² is Cl, R⁷ is CF₃ and the combination of R⁵ and R⁶ for a compound corresponds in each case to one row of Table A;

Table 5 Compounds of the formula (IA-1) in which R¹ is F, R² is Br, R⁷ is CF₃ and the combination of R⁵ and R⁶ for a compound corresponds in each case to one row of Table A;

Table 6 Compounds of the formula (IA-1) in which R¹ is Br, R² is Br, R⁷ is CF₃ and the combination of R⁵ and R⁶ for a compound corresponds in each case to one row of Table A;

Table 7 Compounds of the formula (IA-1) in which R¹ is Cl, R² is Br, R⁷ is CF₃ and the combination of R⁵ and R⁶ for a compound corresponds in each case to one row of Table A;

Table 8 Compounds of the formula (IA-1) in which R¹ is methyl, R² is Br, R⁷ is CF₃ and the combination of R⁵ and R⁶ for a compound corresponds in each case to one row of Table A;

Table 9 Compounds of the formula (IA-1) in which R¹ is F, R² is cyano, R⁷ is CF₃ and the combination of R⁵ and R⁶ for a compound corresponds in each case to one row of Table A;

Table 10 Compounds of the formula (IA-1) in which R¹ is Br, R² is cyano, R⁷ is CF₃ and the combination of R⁵ and R⁶ for a compound corresponds in each case to one row of Table A;

Table 11 Compounds of the formula (IA-1) in which R¹ is Cl, R² is cyano, R⁷ is CF₃ and the combination of R⁵ and R⁶ for a compound corresponds in each case to one row of Table A;

Table 12 Compounds of the formula (IA-1) in which R¹ is methyl, R² is cyano, R⁷ is CF₃ and the combination of R⁵ and R⁶ for a compound corresponds in each case to one row of Table A;

Table 13 Compounds of the formula (IA-1) in which R¹ is F, R² is Cl, R⁷ is CHF₂ and the combination of R⁵ and R⁶ for a compound corresponds in each case to one row of Table A;

Table 14 Compounds of the formula (IA-1) in which R¹ is Br, R² is Cl, R⁷ is CHF₂ and the combination of R⁵ and R⁶ for a compound corresponds in each case to one row of Table A;

Table 15 Compounds of the formula (IA-1) in which R¹ is Cl, R² is Cl, R⁷ is CHF₂ and the combination of R⁵ and R⁶ for a compound corresponds in each case to one row of Table A;

Table 16 Compounds of the formula (IA-1) in which R¹ is methyl, R² is Cl, R⁷ is CHF₂ and the combination of R⁵ and R⁶ for a compound corresponds in each case to one row of Table A;

Table 17 Compounds of the formula (IA-1) in which R¹ is F, R² is Br, R⁷ is CHF₂ and the combination of R⁵ and R⁶ for a compound corresponds in each case to one row of Table A;

Table 18 Compounds of the formula (IA-1) in which R¹ is Br, R² is Br, R⁷ is CHF₂ and the combination of R⁵ and R⁶ for a compound corresponds in each case to one row of Table A;

Table 19 Compounds of the formula (IA-1) in which R¹ is Cl, R² is Br, R⁷ is CHF₂ and the combination of R⁵ and R⁶ for a compound corresponds in each case to one row of Table A;

Table 20 Compounds of the formula (IA-1) in which R¹ is methyl, R² is Br, R⁷ is CHF₂ and the combination of R⁵ and R⁶ for a compound corresponds in each case to one row of Table A;

Table 21 Compounds of the formula (IA-1) in which R¹ is F, R² is cyano, R⁷ is CHF₂ and the combination of R⁵ and R⁶ for a compound corresponds in each case to one row of Table A;

Table 22 Compounds of the formula (IA-1) in which R¹ is Br, R² is cyano, R⁷ is CHF₂ and the combination of R⁵ and R⁶ for a compound corresponds in each case to one row of Table A;

Table 23 Compounds of the formula (IA-1) in which R¹ is Cl, R² is cyano, R⁷ is CHF₂ and the combination of R⁵ and R⁶ for a compound corresponds in each case to one row of Table A;

Table 24 Compounds of the formula (IA-1) in which R¹ is methyl, R² is cyano, R⁷ is CHF₂ and the combination of R⁵ and R⁶ for a compound corresponds in each case to one row of Table A;

Table 25 Compounds of the formula (IA-1) in which R¹ is F, R² is Cl, R⁷ is Br and the combination of R⁵ and R⁶ for a compound corresponds in each case to one row of Table A;

Table 26 Compounds of the formula (IA-1) in which R¹ is Br, R² is Cl, R⁷ is Br and the combination of R⁵ and R⁶ for a compound corresponds in each case to one row of Table A;

Table 27 Compounds of the formula (IA-1) in which R¹ is Cl, R² is Cl, R⁷ is Br and the combination of R⁵ and R⁶ for a compound corresponds in each case to one row of Table A;

Table 28 Compounds of the formula (IA-1) in which R¹ is methyl, R² is Cl, R⁷ is Br and the combination of R⁵ and R⁶ for a compound corresponds in each case to one row of Table A;

Table 29 Compounds of the formula (IA-1) in which R¹ is F, R² is Br, R⁷ is Br and the combination of R⁵ and R⁶ for a compound corresponds in each case to one row of Table A;

Table 30 Compounds of the formula (IA-1) in which R¹ is Br, R² is Br, R⁷ is Br and the combination of R⁵ and R⁶ for a compound corresponds in each case to one row of Table A;

Table 31 Compounds of the formula (IA-1) in which R¹ is Cl, R² is Br, R⁷ is Br and the combination of R⁵ and R⁶ for a compound corresponds in each case to one row of Table A;

Table 32 Compounds of the formula (IA-1) in which R¹ is methyl, R² is Br, R⁷ is Br and the combination of R⁵ and R⁶ for a compound corresponds in each case to one row of Table A;

Table 33 Compounds of the formula (IA-1) in which R¹ is F, R² is cyano, R⁷ is Br and the combination of R⁵ and R⁶ for a compound corresponds in each case to one row of Table A;

Table 34 Compounds of the formula (IA-1) in which R¹ is Br, R² is cyano, R⁷ is Br and the combination of R⁵ and R⁶ for a compound corresponds in each case to one row of Table A;

Table 35 Compounds of the formula (IA-1) in which R¹ is Cl, R² is cyano, R⁷ is Br and the combination of R⁵ and R⁶ for a compound corresponds in each case to one row of Table A;

Table 36 Compounds of the formula (IA-1) in which R¹ is methyl, R² is cyano, R⁷ is Br and the combination of R⁵ and R⁶ for a compound corresponds in each case to one row of Table A;

Table 37 Compounds of the formula (IA-1) in which R¹ is F, R² is Cl, R⁷ is Cl and the combination of R⁵ and R⁶ for a compound corresponds in each case to one row of Table A;

Table 38 Compounds of the formula (IA-1) in which R¹ is Br, R² is Cl, R⁷ is Cl and the combination of R⁵ and R⁶ for a compound corresponds in each case to one row of Table A;

Table 39 Compounds of the formula (IA-1) in which R¹ is Cl, R² is Cl, R⁷ is Cl and the combination of R⁵ and R⁶ for a compound corresponds in each case to one row of Table A;

Table 40 Compounds of the formula (IA-1) in which R¹ is methyl, R² is Cl, R⁷ is Cl and the combination of R⁵ and R⁶ for a compound corresponds in each case to one row of Table A;

Table 41 Compounds of the formula (IA-1) in which R¹ is F, R² is Br, R⁷ is Cl and the combination of R⁵ and R⁶ for a compound corresponds in each case to one row of Table A;

Table 42 Compounds of the formula (IA-1) in which R¹ is Br, R² is Br, R⁷ is Cl and the combination of R⁵ and R⁶ for a compound corresponds in each case to one row of Table A;

Table 43 Compounds of the formula (IA-1) in which R¹ is Cl, R² is Br, R⁷ is Cl and the combination of R⁵ and R⁶ for a compound corresponds in each case to one row of Table A;

Table 44 Compounds of the formula (IA-1) in which R¹ is methyl, R² is Br, R⁷ is Cl and the combination of R⁵ and R⁶ for a compound corresponds in each case to one row of Table A;

Table 45 Compounds of the formula (IA-1) in which R¹ is F, R² is cyano, R⁷ is Cl and the combination of R⁵ and R⁶ for a compound corresponds in each case to one row of Table A;

Table 46 Compounds of the formula (IA-1) in which R¹ is Br, R² is cyano, R⁷ is Cl and the combination of R⁵ and R⁶ for a compound corresponds in each case to one row of Table A;

Table 47 Compounds of the formula (IA-1) in which R¹ is Cl, R² is cyano, R⁷ is Cl and the combination of R⁵ and R⁶ for a compound corresponds in each case to one row of Table A;

Table 48 Compounds of the formula (IA-1) in which R¹ is methyl, R² is cyano, R⁷ is Cl and the combination of R⁵ and R⁶ for a compound corresponds in each case to one row of Table A;

Table 49 Compounds of the formula (IA-1) in which R¹ is F, R² is Cl, R⁷ is OCHF₂ and the combination of R⁵ and R⁶ for a compound corresponds in each case to one row of Table A;

Table 50 Compounds of the formula (IA-1) in which R¹ is Br, R² is Cl, R⁷ is OCHF₂ and the combination of R⁵ and R⁶ for a compound corresponds in each case to one row of Table A;

Table 51 Compounds of the formula (IA-1) in which R¹ is Cl, R² is Cl, R⁷ is OCHF₂ and the combination of R⁵ and R⁶ for a compound corresponds in each case to one row of Table A;

Table 52 Compounds of the formula (IA-1) in which R¹ is methyl, R² is Cl, R⁷ is OCHF₂ and the combination of R⁵ and R⁶ for a compound corresponds in each case to one row of Table A;

Table 53 Compounds of the formula (IA-1) in which R¹ is F, R² is Br, R⁷ is OCHF₂ and the combination of R⁵ and R⁶ for a compound corresponds in each case to one row of Table A;

Table 54 Compounds of the formula (IA-1) in which R¹ is Br, R² is Br, R⁷ is OCHF₂ and the combination of R⁵ and R⁶ for a compound corresponds in each case to one row of Table A;

Table 55 Compounds of the formula (IA-1) in which R¹ is Cl, R² is Br, R⁷ is OCHF₂ and the combination of R⁵ and R⁶ for a compound corresponds in each case to one row of Table A;

Table 56 Compounds of the formula (IA-1) in which R¹ is methyl, R² is Br, R⁷ is OCHF₂ and the combination of R⁵ and R⁶ for a compound corresponds in each case to one row of Table A;

Table 57 Compounds of the formula (IA-1) in which R¹ is F, R² is cyano, R⁷ is OCHF₂ and the combination of R⁵ and R⁶ for a compound corresponds in each case to one row of Table A;

Table 58 Compounds of the formula (IA-1) in which R¹ is Br, R² is cyano, R⁷ is OCHF₂ and the combination of R⁵ and R⁶ for a compound corresponds in each case to one row of Table A;

Table 59 Compounds of the formula (IA-1) in which R¹ is Cl, R² is cyano, R⁷ is OCHF₂ and the combination of R⁵ and R⁶ for a compound corresponds in each case to one row of Table A;

Table 60 Compounds of the formula (IA-1) in which R¹ is methyl, R² is cyano, R⁷ is OCHF₂ and the combination of R⁵ and R⁶ for a compound corresponds in each case to one row of Table A.

TABLE A

| | R⁵ | R⁶ |
|---|---|---|
| A-1 | CH₃ | CH₃ |
| A-2 | C₂H₅ | CH₃ |
| A-3 | CH═CH₂ | CH₃ |
| A-4 | CH₂CH₂CH₃ | CH₃ |
| A-5 | CH(CH₃)₂ | CH₃ |
| A-6 | CH₂CH₂CH₂CH₃ | CH₃ |
| A-7 | C(CH₃)₃ | CH₃ |
| A-8 | CH₂CH(CH₃)₂ | CH₃ |
| A-9 | CH(CH₃)CH₂CH₃ | CH₃ |
| A-10 | CH₂CH═CH₂ | CH₃ |
| A-11 | CH₂C≡CH | CH₃ |
| A-12 | CH(CH₃)CH═CH₂ | CH₃ |
| A-13 | CHF₂ | CH₃ |
| A-14 | CH₂Cl | CH₃ |
| A-15 | CH₂CH₂CN | CH₃ |
| A-16 | CH₂CH₂Cl | CH₃ |
| A-17 | c-C₃H₅ | CH₃ |
| A-18 | c-C₄H₇ | CH₃ |
| A-19 | c-C₅H₉ | CH₃ |
| A-20 | c-C₆H₁₁ | CH₃ |
| A-21 | CH₂—c-C₃H₅ | CH₃ |
| A-22 | CH(CH₃)—c-C₃H₅ | CH₃ |
| A-23 | CH₂—c-C₅H₉ | CH₃ |
| A-24 | CH₂—c-C₆H₁₁ | CH₃ |
| A-25 | C₆H₅ | CH₃ |
| A-26 | CH₃ | C₂H₅ |
| A-27 | C₂H₅ | C₂H₅ |
| A-28 | CH═CH₂ | C₂H₅ |
| A-29 | CH₂CH₂CH₃ | C₂H₅ |
| A-30 | CH(CH₃)₂ | C₂H₅ |
| A-31 | CH₂CH₂CH₂CH₃ | C₂H₅ |
| A-32 | C(CH₃)₃ | C₂H₅ |
| A-33 | CH₂CH(CH₃)₂ | C₂H₅ |
| A-34 | CH(CH₃)CH₂CH₃ | C₂H₅ |
| A-35 | CH₂CH═CH₂ | C₂H₅ |
| A-36 | CH₂C≡CH | C₂H₅ |
| A-37 | CH(CH₃)CH═CH₂ | C₂H₅ |
| A-38 | CHF₂ | C₂H₅ |
| A-39 | CH₂Cl | C₂H₅ |
| A-40 | CH₂CH₂CN | C₂H₅ |
| A-41 | CH₂CH₂Cl | C₂H₅ |
| A-42 | c-C₃H₅ | C₂H₅ |
| A-43 | c-C₄H₇ | C₂H₅ |
| A-44 | c-C₅H₉ | C₂H₅ |
| A-45 | c-C₆H₁₁ | C₂H₅ |
| A-46 | CH₂—c-C₃H₅ | C₂H₅ |
| A-47 | CH(CH₃)—c-C₃H₅ | C₂H₅ |
| A-48 | CH₂—c-C₅H₉ | C₂H₅ |
| A-49 | CH₂—c-C₆H₁₁ | C₂H₅ |
| A-50 | C₆H₅ | C₂H₅ |
| A-51 | CH₃ | CH═CH₂ |
| A-52 | C₂H₅ | CH═CH₂ |
| A-53 | CH═CH₂ | CH═CH₂ |
| A-54 | CH₂CH₂CH₃ | CH═CH₂ |
| A-55 | CH(CH₃)₂ | CH═CH₂ |
| A-56 | CH₂CH₂CH₂CH₃ | CH═CH₂ |
| A-57 | C(CH₃)₃ | CH═CH₂ |
| A-58 | CH₂CH(CH₃)₂ | CH═CH₂ |
| A-59 | CH(CH₃)CH₂CH₃ | CH═CH₂ |
| A-60 | CH₂CH═CH₂ | CH═CH₂ |
| A-61 | CH₂C≡CH | CH═CH₂ |
| A-62 | CH(CH₃)CH═CH₂ | CH═CH₂ |
| A-63 | CHF₂ | CH═CH₂ |
| A-64 | CH₂Cl | CH═CH₂ |
| A-65 | CH₂CH₂CN | CH═CH₂ |
| A-66 | CH₂CH₂Cl | CH═CH₂ |
| A-67 | c-C₃H₅ | CH═CH₂ |
| A-68 | c-C₄H₇ | CH═CH₂ |
| A-69 | c-C₅H₉ | CH═CH₂ |
| A-70 | c-C₆H₁₁ | CH═CH₂ |
| A-71 | CH₂—c-C₃H₅ | CH═CH₂ |
| A-72 | CH(CH₃)—c-C₃H₅ | CH═CH₂ |
| A-73 | CH₂—c-C₅H₉ | CH═CH₂ |
| A-74 | CH₂—c-C₆H₁₁ | CH═CH₂ |
| A-75 | C₆H₅ | CH═CH₂ |
| A-76 | CH₃ | CH₂CH₂CH₃ |
| A-77 | C₂H₅ | CH₂CH₂CH₃ |
| A-78 | CH═CH₂ | CH₂CH₂CH₃ |
| A-79 | CH₂CH₂CH₃ | CH₂CH₂CH₃ |
| A-80 | CH(CH₃)₂ | CH₂CH₂CH₃ |
| A-81 | CH₂CH₂CH₂CH₃ | CH₂CH₂CH₃ |
| A-82 | C(CH₃)₃ | CH₂CH₂CH₃ |
| A-83 | CH₂CH(CH₃)₂ | CH₂CH₂CH₃ |
| A-84 | CH(CH₃)CH₂CH₃ | CH₂CH₂CH₃ |
| A-85 | CH₂CH═CH₂ | CH₂CH₂CH₃ |
| A-86 | CH₂C≡CH | CH₂CH₂CH₃ |
| A-87 | CH(CH₃)CH═CH₂ | CH₂CH₂CH₃ |
| A-88 | CHF₂ | CH₂CH₂CH₃ |
| A-89 | CH₂Cl | CH₂CH₂CH₃ |
| A-90 | CH₂CH₂CN | CH₂CH₂CH₃ |
| A-91 | CH₂CH₂Cl | CH₂CH₂CH₃ |
| A-92 | c-C₃H₅ | CH₂CH₂CH₃ |
| A-93 | c-C₄H₇ | CH₂CH₂CH₃ |
| A-94 | c-C₅H₉ | CH₂CH₂CH₃ |
| A-95 | c-C₆H₁₁ | CH₂CH₂CH₃ |
| A-96 | CH₂—c-C₃H₅ | CH₂CH₂CH₃ |
| A-97 | CH(CH₃)—c-C₃H₅ | CH₂CH₂CH₃ |
| A-98 | CH₂—c-C₅H₉ | CH₂CH₂CH₃ |
| A-99 | CH₂—c-C₆H₁₁ | CH₂CH₂CH₃ |
| A-100 | C₆H₅ | CH₂CH₂CH₃ |
| A-101 | CH₃ | CH(CH₃)₂ |
| A-102 | C₂H₅ | CH(CH₃)₂ |
| A-103 | CH═CH₂ | CH(CH₃)₂ |
| A-104 | CH₂CH₂CH₃ | CH(CH₃)₂ |
| A-105 | CH(CH₃)₂ | CH(CH₃)₂ |
| A-106 | CH₂CH₂CH₂CH₃ | CH(CH₃)₂ |
| A-107 | C(CH₃)₃ | CH(CH₃)₂ |
| A-108 | CH₂CH(CH₃)₂ | CH(CH₃)₂ |
| A-109 | CH(CH₃)CH₂CH₃ | CH(CH₃)₂ |
| A-110 | CH₂CH═CH₂ | CH(CH₃)₂ |
| A-111 | CH₂C≡CH | CH(CH₃)₂ |
| A-112 | CH(CH₃)CH═CH₂ | CH(CH₃)₂ |
| A-113 | CHF₂ | CH(CH₃)₂ |
| A-114 | CH₂Cl | CH(CH₃)₂ |
| A-115 | CH₂CH₂CN | CH(CH₃)₂ |
| A-116 | CH₂CH₂Cl | CH(CH₃)₂ |
| A-117 | c-C₃H₅ | CH(CH₃)₂ |
| A-118 | c-C₄H₇ | CH(CH₃)₂ |
| A-119 | c-C₅H₉ | CH(CH₃)₂ |
| A-120 | c-C₆H₁₁ | CH(CH₃)₂ |
| A-121 | CH₂—c-C₃H₅ | CH(CH₃)₂ |
| A-122 | CH(CH₃)—c-C₃H₅ | CH(CH₃)₂ |
| A-123 | CH₂—c-C₅H₉ | CH(CH₃)₂ |
| A-124 | CH₂—c-C₆H₁₁ | CH(CH₃)₂ |
| A-125 | C₆H₅ | CH(CH₃)₂ |
| A-126 | CH₃ | CH₂CH₂CH₂CH₃ |
| A-127 | C₂H₅ | CH₂CH₂CH₂CH₃ |
| A-128 | CH═CH₂ | CH₂CH₂CH₂CH₃ |
| A-129 | CH₂CH₂CH₃ | CH₂CH₂CH₂CH₃ |
| A-130 | CH(CH₃)₂ | CH₂CH₂CH₂CH₃ |
| A-131 | CH₂CH₂CH₂CH₃ | CH₂CH₂CH₂CH₃ |
| A-132 | C(CH₃)₃ | CH₂CH₂CH₂CH₃ |
| A-133 | CH₂CH(CH₃)₂ | CH₂CH₂CH₂CH₃ |
| A-134 | CH(CH₃)CH₂CH₃ | CH₂CH₂CH₂CH₃ |
| A-135 | CH₂CH═CH₂ | CH₂CH₂CH₂CH₃ |
| A-136 | CH₂C≡CH | CH₂CH₂CH₂CH₃ |
| A-137 | CH(CH₃)CH═CH₂ | CH₂CH₂CH₂CH₃ |
| A-138 | CHF₂ | CH₂CH₂CH₂CH₃ |
| A-139 | CH₂Cl | CH₂CH₂CH₂CH₃ |
| A-140 | CH₂CH₂CN | CH₂CH₂CH₂CH₃ |
| A-141 | CH₂CH₂Cl | CH₂CH₂CH₂CH₃ |
| A-142 | c-C₃H₅ | CH₂CH₂CH₂CH₃ |
| A-143 | c-C₄H₇ | CH₂CH₂CH₂CH₃ |
| A-144 | c-C₅H₉ | CH₂CH₂CH₂CH₃ |
| A-145 | c-C₆H₁₁ | CH₂CH₂CH₂CH₃ |
| A-146 | CH₂—c-C₃H₅ | CH₂CH₂CH₂CH₃ |
| A-147 | CH(CH₃)—c-C₃H₅ | CH₂CH₂CH₂CH₃ |
| A-148 | CH₂—c-C₅H₉ | CH₂CH₂CH₂CH₃ |
| A-149 | CH₂—c-C₆H₁₁ | CH₂CH₂CH₂CH₃ |
| A-150 | C₆H₅ | CH₂CH₂CH₂CH₃ |
| A-151 | CH₃ | C(CH₃)₃ |
| A-152 | C₂H₅ | C(CH₃)₃ |
| A-153 | CH═CH₂ | C(CH₃)₃ |
| A-154 | CH₂CH₂CH₃ | C(CH₃)₃ |
| A-155 | CH(CH₃)₂ | C(CH₃)₃ |
| A-156 | CH₂CH₂CH₂CH₃ | C(CH₃)₃ |
| A-157 | C(CH₃)₃ | C(CH₃)₃ |
| A-158 | CH₂CH(CH₃)₂ | C(CH₃)₃ |
| A-159 | CH(CH₃)CH₂CH₃ | C(CH₃)₃ |
| A-160 | CH₂CH═CH₂ | C(CH₃)₃ |
| A-161 | CH₂C≡CH | C(CH₃)₃ |
| A-162 | CH(CH₃)CH═CH₂ | C(CH₃)₃ |
| A-163 | CHF₂ | C(CH₃)₃ |
| A-164 | CH₂Cl | C(CH₃)₃ |
| A-165 | CH₂CH₂CN | C(CH₃)₃ |
| A-166 | CH₂CH₂Cl | C(CH₃)₃ |
| A-167 | c-C₃H₅ | C(CH₃)₃ |
| A-168 | c-C₄H₇ | C(CH₃)₃ |
| A-169 | c-C₅H₉ | C(CH₃)₃ |
| A-170 | c-C₆H₁₁ | C(CH₃)₃ |
| A-171 | CH₂—c-C₃H₅ | C(CH₃)₃ |
| A-172 | CH(CH₃)—c-C₃H₅ | C(CH₃)₃ |
| A-173 | CH₂—c-C₅H₉ | C(CH₃)₃ |
| A-174 | CH₂—c-C₆H₁₁ | C(CH₃)₃ |
| A-175 | C₆H₅ | C(CH₃)₃ |
| A-176 | CH₃ | CH₂CH(CH₃)₂ |
| A-177 | C₂H₅ | CH₂CH(CH₃)₂ |
| A-178 | CH═CH₂ | CH₂CH(CH₃)₂ |
| A-179 | CH₂CH₂CH₃ | CH₂CH(CH₃)₂ |
| A-180 | CH(CH₃)₂ | CH₂CH(CH₃)₂ |
| A-181 | CH₂CH₂CH₂CH₃ | CH₂CH(CH₃)₂ |
| A-182 | C(CH₃)₃ | CH₂CH(CH₃)₂ |
| A-183 | CH₂CH(CH₃)₂ | CH₂CH(CH₃)₂ |
| A-184 | CH(CH₃)CH₂CH₃ | CH₂CH(CH₃)₂ |
| A-185 | CH₂CH═CH₂ | CH₂CH(CH₃)₂ |
| A-186 | CH₂C≡CH | CH₂CH(CH₃)₂ |
| A-187 | CH(CH₃)CH═CH₂ | CH₂CH(CH₃)₂ |
| A-188 | CHF₂ | CH₂CH(CH₃)₂ |
| A-189 | CH₂Cl | CH₂CH(CH₃)₂ |
| A-190 | CH₂CH₂CN | CH₂CH(CH₃)₂ |
| A-191 | CH₂CH₂Cl | CH₂CH(CH₃)₂ |
| A-192 | c-C₃H₅ | CH₂CH(CH₃)₂ |
| A-193 | c-C₄H₇ | CH₂CH(CH₃)₂ |
| A-194 | c-C₅H₉ | CH₂CH(CH₃)₂ |
| A-195 | c-C₆H₁₁ | CH₂CH(CH₃)₂ |
| A-196 | CH₂—c-C₃H₅ | CH₂CH(CH₃)₂ |
| A-197 | CH(CH₃)—c-C₃H₅ | CH₂CH(CH₃)₂ |
| A-198 | CH₂—c-C₅H₉ | CH₂CH(CH₃)₂ |
| A-199 | CH₂—c-C₆H₁₁ | CH₂CH(CH₃)₂ |
| A-200 | C₆H₅ | CH₂CH(CH₃)₂ |
| A-201 | CH₃ | CH(CH₃)CH₂CH₃ |
| A-202 | C₂H₅ | CH(CH₃)CH₂CH₃ |
| A-203 | CH═CH₂ | CH(CH₃)CH₂CH₃ |
| A-204 | CH₂CH₂CH₃ | CH(CH₃)CH₂CH₃ |
| A-205 | CH(CH₃)₂ | CH(CH₃)CH₂CH₃ |
| A-206 | CH₂CH₂CH₂CH₃ | CH(CH₃)CH₂CH₃ |
| A-207 | C(CH₃)₃ | CH(CH₃)CH₂CH₃ |
| A-208 | CH₂CH(CH₃)₂ | CH(CH₃)CH₂CH₃ |
| A-209 | CH(CH₃)CH₂CH₃ | CH(CH₃)CH₂CH₃ |
| A-210 | CH₂CH═CH₂ | CH(CH₃)CH₂CH₃ |
| A-211 | CH₂C≡CH | CH(CH₃)CH₂CH₃ |
| A-212 | CH(CH₃)CH═CH₂ | CH(CH₃)CH₂CH₃ |
| A-213 | CHF₂ | CH(CH₃)CH₂CH₃ |
| A-214 | CH₂Cl | CH(CH₃)CH₂CH₃ |
| A-215 | CH₂CH₂CN | CH(CH₃)CH₂CH₃ |
| A-216 | CH₂CH₂Cl | CH(CH₃)CH₂CH₃ |
| A-217 | c-C₃H₅ | CH(CH₃)CH₂CH₃ |
| A-218 | c-C₄H₇ | CH(CH₃)CH₂CH₃ |
| A-219 | c-C₅H₉ | CH(CH₃)CH₂CH₃ |
| A-220 | c-C₆H₁₁ | CH(CH₃)CH₂CH₃ |
| A-221 | CH₂—c-C₃H₅ | CH(CH₃)CH₂CH₃ |
| A-222 | CH(CH₃)—c-C₃H₅ | CH(CH₃)CH₂CH₃ |
| A-223 | CH₂—c-C₅H₉ | CH(CH₃)CH₂CH₃ |
| A-224 | CH₂—c-C₆H₁₁ | CH(CH₃)CH₂CH₃ |
| A-225 | C₆H₅ | CH(CH₃)CH₂CH₃ |
| A-226 | CH₃ | CH₂CH═CH₂ |
| A-227 | C₂H₅ | CH₂CH═CH₂ |
| A-228 | CH═CH₂ | CH₂CH═CH₂ |
| A-229 | CH₂CH₂CH₃ | CH₂CH═CH₂ |
| A-230 | CH(CH₃)₂ | CH₂CH═CH₂ |
| A-231 | CH₂CH₂CH₂CH₃ | CH₂CH═CH₂ |
| A-232 | C(CH₃)₃ | CH₂CH═CH₂ |
| A-233 | CH₂CH(CH₃)₂ | CH₂CH═CH₂ |
| A-234 | CH(CH₃)CH₂CH₃ | CH₂CH═CH₂ |
| A-235 | CH₂CH═CH₂ | CH₂CH═CH₂ |
| A-236 | CH₂C≡CH | CH₂CH═CH₂ |
| A-237 | CH(CH₃)CH═CH₂ | CH₂CH═CH₂ |
| A-238 | CHF₂ | CH₂CH═CH₂ |
| A-239 | CH₂Cl | CH₂CH═CH₂ |
| A-240 | CH₂CH₂CN | CH₂CH═CH₂ |
| A-241 | CH₂CH₂Cl | CH₂CH═CH₂ |
| A-242 | c-C₃H₅ | CH₂CH═CH₂ |
| A-243 | c-C₄H₇ | CH₂CH═CH₂ |
| A-244 | c-C₅H₉ | CH₂CH═CH₂ |
| A-245 | c-C₆H₁₁ | CH₂CH═CH₂ |
| A-246 | CH₂—c-C₃H₅ | CH₂CH═CH₂ |
| A-247 | CH(CH₃)—c-C₃H₅ | CH₂CH═CH₂ |
| A-248 | CH₂—c-C₅H₉ | CH₂CH═CH₂ |
| A-249 | CH₂—c-C₆H₁₁ | CH₂CH═CH₂ |
| A-250 | C₆H₅ | CH₂CH═CH₂ |
| A-251 | CH₃ | CH₂C≡CH |
| A-252 | C₂H₅ | CH₂C≡CH |
| A-253 | CH═CH₂ | CH₂C≡CH |
| A-254 | CH₂CH₂CH₃ | CH₂C≡CH |
| A-255 | CH(CH₃)₂ | CH₂C≡CH |
| A-256 | CH₂CH₂CH₂CH₃ | CH₂C≡CH |
| A-257 | C(CH₃)₃ | CH₂C≡CH |
| A-258 | CH₂CH(CH₃)₂ | CH₂C≡CH |
| A-259 | CH(CH₃)CH₂CH₃ | CH₂C≡CH |
| A-260 | CH₂CH═CH₂ | CH₂C≡CH |
| A-261 | CH2C≡CH | CH₂C≡CH |
| A-262 | CH(CH₃)CH═CH₂ | CH₂C≡CH |
| A-263 | CHF₂ | CH₂C≡CH |
| A-264 | CH₂Cl | CH₂C≡CH |
| A-265 | CH₂CH₂CN | CH₂C≡CH |
| A-266 | CH₂CH₂Cl | CH₂C≡CH |
| A-267 | c-C₃H₅ | CH₂C≡CH |
| A-268 | c-C₄H₇ | CH₂C≡CH |
| A-269 | c-C₅H₉ | CH₂C≡CH |
| A-270 | c-C₆H₁₁ | CH₂C≡CH |
| A-271 | CH₂—c-C₃H₅ | CH₂C≡CH |
| A-272 | CH(CH₃)—c-C₃H₅ | CH₂C≡CH |
| A-273 | CH₂—c-C₅H₉ | CH₂C≡CH |
| A-274 | CH₂—c-C₆H₁₁ | CH₂C≡CH |
| A-275 | C₆H₅ | CH₂C≡CH |
| A-276 | CH₃ | CH(CH₃)CH═CH₂ |
| A-277 | C₂H₅ | CH(CH₃)CH═CH₂ |
| A-278 | CH═CH₂ | CH(CH₃)CH═CH₂ |
| A-279 | CH₂CH₂CH₃ | CH(CH₃)CH═CH₂ |
| A-280 | CH(CH₃)₂ | CH(CH₃)CH═CH₂ |
| A-281 | CH₂CH₂CH₂CH₃ | CH(CH₃)CH═CH₂ |
| A-282 | C(CH₃)₃ | CH(CH₃)CH═CH₂ |
| A-283 | CH₂CH(CH₃)₂ | CH(CH₃)CH═CH₂ |
| A-284 | CH(CH₃)CH₂CH₃ | CH(CH₃)CH═CH₂ |
| A-285 | CH₂CH═CH₂ | CH(CH₃)CH═CH₂ |
| A-286 | CH₂C≡CH | CH(CH₃)CH═CH₂ |
| A-287 | CH(CH₃)CH═CH₂ | CH(CH₃)CH═CH₂ |
| A-288 | CHF₂ | CH(CH₃)CH═CH₂ |
| A-289 | CH₂Cl | CH(CH₃)CH═CH₂ |
| A-290 | CH₂CH₂CN | CH(CH₃)CH═CH₂ |
| A-291 | CH₂CH₂Cl | CH(CH₃)CH═CH₂ |
| A-292 | c-C₃H₅ | CH(CH₃)CH═CH₂ |
| A-293 | c-C₄H₇ | CH(CH₃)CH═CH₂ |
| A-294 | c-C₅H₉ | CH(CH₃)CH═CH₂ |
| A-295 | c-C₆H₁₁ | CH(CH₃)CH═CH₂ |
| A-296 | CH₂—c-C₃H₅ | CH(CH₃)CH═CH₂ |
| A-297 | CH(CH₃)—c-C₃H₅ | CH(CH₃)CH═CH₂ |
| A-298 | CH₂—c-C₅H₉ | CH(CH₃)CH═CH₂ |
| A-299 | CH₂—c-C₅H₉ | CH(CH₃)CH═CH₂ |
| A-300 | C₆H₅ | CH(CH₃)CH═CH₂ |
| A-301 | CH₃ | CHF₂ |
| A-302 | C₂H₅ | CHF₂ |
| A-303 | CH═CH₂ | CHF₂ |
| A-304 | CH₂CH₂CH₃ | CHF₂ |
| A-305 | CH(CH₃)₂ | CHF₂ |
| A-306 | CH₂CH₂CH₂CH₃ | CHF₂ |
| A-307 | C(CH₃)₃ | CHF₂ |
| A-308 | CH₂CH(CH₃)₂ | CHF₂ |
| A-309 | CH(CH₃)CH₂CH₃ | CHF₂ |
| A-310 | CH₂CH═CH₂ | CHF₂ |
| A-311 | CH₂C≡CH | CHF₂ |
| A-312 | CH(CH₃)CH═CH₂ | CHF₂ |
| A-313 | CHF₂ | CHF₂ |
| A-314 | CH₂Cl | CHF₂ |
| A-315 | CH₂CH₂CN | CHF₂ |
| A-316 | CH₂CH₂Cl | CHF₂ |
| A-317 | c-C₃H₅ | CHF₂ |
| A-318 | c-C₄H₇ | CHF₂ |
| A-319 | c-C₅H₉ | CHF₂ |
| A-320 | c-C₆H₁₁ | CHF₂ |
| A-321 | CH₂—c-C₃H₅ | CHF₂ |
| A-322 | CH(CH₃)—c-C₃H₅ | CHF₂ |
| A-323 | CH₂—c-C₅H₉ | CHF₂ |
| A-324 | CH₂—c-C₆H₁₁ | CHF₂ |
| A-325 | C₆H₅ | CHF₂ |
| A-326 | CH₃ | CH₂Cl |
| A-327 | C₂H₅ | CH₂Cl |
| A-328 | CH═CH₂ | CH₂Cl |
| A-329 | CH₂CH₂CH₃ | CH₂Cl |
| A-330 | CH(CH₃)₂ | CH₂Cl |
| A-331 | CH₂CH₂CH₂CH₃ | CH₂Cl |
| A-332 | C(CH₃)₃ | CH₂Cl |
| A-333 | CH₂CH(CH₃)₂ | CH₂Cl |
| A-334 | CH(CH₃)CH₂CH₃ | CH₂Cl |
| A-335 | CH₂CH═CH₂ | CH₂Cl |
| A-336 | CH₂C≡CH | CH₂Cl |
| A-337 | CH(CH₃)CH═CH₂ | CH₂Cl |
| A-338 | CHF₂ | CH₂Cl |
| A-339 | CH₂Cl | CH₂Cl |
| A-340 | CH₂CH₂CN | CH₂Cl |
| A-341 | CH₂CH₂Cl | CH₂Cl |
| A-342 | c-C₃H₅ | CH₂Cl |
| A-343 | c-C₄H₇ | CH₂Cl |
| A-344 | c-C₅H₉ | CH₂Cl |
| A-345 | c-C₆H₁₁ | CH₂Cl |
| A-346 | CH₂—c-C₃H₅ | CH₂Cl |
| A-347 | CH(CH₃)—c-C₃H₅ | CH₂Cl |
| A-348 | CH₂—c-C₅H₉ | CH₂Cl |
| A-349 | CH₂—c-C₆H₁₁ | CH₂Cl |
| A-350 | C₆H₅ | CH₂Cl |
| A-351 | CH₃ | CH₂CH₂CN |
| A-352 | C₂H₅ | CH₂CH₂CN |
| A-353 | CH═CH₂ | CH₂CH₂CN |
| A-354 | CH₂CH₂CH₃ | CH₂CH₂CN |
| A-355 | CH(CH₃)₂ | CH₂CH₂CN |
| A-356 | CH₂CH₂CH₂CH₃ | CH₂CH₂CN |
| A-357 | C(CH₃)₃ | CH₂CH₂CN |
| A-358 | CH₂CH(CH₃)₂ | CH₂CH₂CN |
| A-359 | CH(CH₃)CH₂CH₃ | CH₂CH₂CN |
| A-360 | CH₂CH═CH₂ | CH₂CH₂CN |
| A-361 | CH₂C≡CH | CH₂CH₂CN |
| A-362 | CH(CH₃)CH═CH₂ | CH₂CH₂CN |
| A-363 | CHF₂ | CH₂CH₂CN |
| A-364 | CH₂Cl | CH₂CH₂CN |
| A-365 | CH₂CH₂CN | CH₂CH₂CN |
| A-366 | CH₂CH₂Cl | CH₂CH₂CN |
| A-367 | c-C₃H₅ | CH₂CH₂CN |
| A-368 | c-C₄H₇ | CH₂CH₂CN |
| A-369 | c-C₅H₉ | CH₂CH₂CN |
| A-370 | c-C₆H₁₁ | CH₂CH₂CN |
| A-371 | CH₂—c-C₃H₅ | CH₂CH₂CN |
| A-372 | CH(CH₃)—c-C₃H₅ | CH₂CH₂CN |
| A-373 | CH₂—c-C₅H₉ | CH₂CH₂CN |
| A-374 | CH₂—c-C₅H₉ | CH₂CH₂CN |
| A-375 | C₆H₅ | CH₂CH₂CN |
| A-376 | CH₃ | CH₂CH₂Cl |
| A-377 | C₂H₅ | CH₂CH₂Cl |
| A-378 | CH═CH₂ | CH₂CH₂Cl |
| A-379 | CH₂CH₂CH₃ | CH₂CH₂Cl |
| A-380 | CH(CH₃)₂ | CH₂CH₂Cl |
| A-381 | CH₂CH₂CH₂CH₃ | CH₂CH₂Cl |
| A-382 | C(CH₃)₃ | CH₂CH₂Cl |
| A-383 | CH₂CH(CH₃)₂ | CH₂CH₂Cl |
| A-384 | CH(CH₃)CH₂CH₃ | CH₂CH₂Cl |
| A-385 | CH₂CH═CH₂ | CH₂CH₂Cl |
| A-386 | CH₂C≡CH | CH₂CH₂Cl |
| A-387 | CH(CH₃)CH═CH₂ | CH₂CH₂Cl |
| A-388 | CHF₂ | CH₂CH₂Cl |
| A-389 | CH₂Cl | CH₂CH₂Cl |
| A-390 | CH₂CH₂CN | CH₂CH₂Cl |
| A-391 | CH₂CH₂Cl | CH₂CH₂Cl |
| A-392 | c-C₃H₅ | CH₂CH₂Cl |
| A-393 | c-C₄H₇ | CH₂CH₂Cl |
| A-394 | c-C₅H₉ | CH₂CH₂Cl |
| A-395 | c-C₆H₁₁ | CH₂CH₂Cl |
| A-396 | CH₂—c-C₃H₅ | CH₂CH₂Cl |
| A-397 | CH(CH₃)—c-C₃H₅ | CH₂CH₂Cl |
| A-398 | CH₂—c-C₅H₉ | CH₂CH₂Cl |
| A-399 | CH₂—c-C₆H₁₁ | CH₂CH₂Cl |
| A-400 | C₆H₅ | CH₂CH₂Cl |
| A-401 | CH₃ | c-C₃H₅ |
| A-402 | C₂H₅ | c-C₃H₅ |
| A-403 | CH═CH₂ | c-C₃H₅ |
| A-404 | CH₂CH₂CH₃ | c-C₃H₅ |
| A-405 | CH(CH₃)₂ | c-C₃H₅ |
| A-406 | CH₂CH₂CH₂CH₃ | c-C₃H₅ |
| A-407 | C(CH₃)₃ | c-C₃H₅ |
| A-408 | CH₂CH(CH₃)₂ | c-C₃H₅ |
| A-409 | CH(CH₃)CH₂CH₃ | c-C₃H₅ |
| A-410 | CH₂CH═CH₂ | c-C₃H₅ |
| A-411 | CH₂C≡CH | c-C₃H₅ |
| A-412 | CH(CH₃)CH═CH₂ | c-C₃H₅ |
| A-413 | CHF₂ | c-C₃H₅ |
| A-414 | CH₂Cl | c-C₃H₅ |
| A-415 | CH₂CH₂CN | c-C₃H₅ |
| A-416 | CH₂CH₂Cl | c-C₃H₅ |
| A-417 | c-C₃H₅ | c-C₃H₅ |
| A-418 | c-C₄H₇ | c-C₃H₅ |
| A-419 | c-C₅H₉ | c-C₃H₅ |
| A-420 | c-C₆H₁₁ | c-C₃H₅ |
| A-421 | CH₂—c-C₃H₅ | c-C₃H₅ |
| A-422 | CH(CH₃)—c-C₃H₅ | c-C₃H₅ |
| A-423 | CH₂—c-C₅H₉ | c-C₃H₅ |
| A-424 | CH₂—c-C₆H₁₁ | c-C₃H₅ |
| A-425 | C₆H₅ | c-C₃H₅ |
| A-426 | CH₃ | c-C₄H₇ |
| A-427 | C₂H₅ | c-C₄H₇ |
| A-428 | CH═CH₂ | c-C₄H₇ |
| A-429 | CH₂CH₂CH₃ | c-C₄H₇ |
| A-430 | CH(CH₃)₂ | c-C₄H₇ |
| A-431 | CH₂CH₂CH₂CH₃ | c-C₄H₇ |
| A-432 | C(CH₃)₃ | c-C₄H₇ |
| A-433 | CH₂CH(CH₃)₂ | c-C₄H₇ |
| A-434 | CH(CH₃)CH₂CH₃ | c-C₄H₇ |
| A-435 | CH₂CH═CH₂ | c-C₄H₇ |
| A-436 | CH₂C≡CH | c-C₄H₇ |
| A-437 | CH(CH₃)CH═CH₂ | c-C₄H₇ |
| A-438 | CHF₂ | c-C₄H₇ |
| A-439 | CH₂Cl | c-C₄H₇ |
| A-440 | CH₂CH₂CN | c-C₄H₇ |
| A-441 | CH₂CH₂Cl | c-C₄H₇ |
| A-442 | c-C₃H₅ | c-C₄H₇ |
| A-443 | c-C₄H₇ | c-C₄H₇ |
| A-444 | c-C₅H₉ | c-C₄H₇ |
| A-445 | c-C₆H₁₁ | c-C₄H₇ |
| A-446 | CH₂—c-C₃H₅ | c-C₄H₇ |
| A-447 | CH(CH₃)—c-C₃H₅ | c-C₄H₇ |
| A-448 | CH₂—c-C₅H₉ | c-C₄H₇ |
| A-449 | CH₂—c-C₆H₁₁ | c-C₄H₇ |
| A-450 | C₆H₅ | c-C₄H₇ |
| A-451 | CH₃ | c-C₅H₉ |
| A-452 | C₂H₅ | c-C₅H₉ |
| A-453 | CH═CH₂ | c-C₅H₉ |
| A-454 | CH₂CH₂CH₃ | c-C₅H₉ |
| A-455 | CH(CH₃)₂ | c-C₅H₉ |
| A-456 | CH₂CH₂CH₂CH₃ | c-C₅H₉ |
| A-457 | C(CH₃)₃ | c-C₅H₉ |
| A-458 | CH₂CH(CH₃)₂ | c-C₅H₉ |
| A-459 | CH(CH₃)CH₂CH₃ | c-C₅H₉ |
| A-460 | CH₂CH═CH₂ | c-C₅H₉ |
| A-461 | CH₂C≡CH | c-C₅H₉ |
| A-462 | CH(CH₃)CH═CH₂ | c-C₅H₉ |
| A-463 | CHF₂ | c-C₅H₉ |
| A-464 | CH₂Cl | c-C₅H₉ |
| A-465 | CH₂CH₂CN | c-C₅H₉ |
| A-466 | CH₂CH₂Cl | c-C₅H₉ |
| A-467 | c-C₃H₅ | c-C₅H₉ |
| A-468 | c-C₄H₇ | c-C₅H₉ |
| A-469 | c-C₅H₉ | c-C₅H₉ |
| A-470 | c-C₆H₁₁ | c-C₅H₉ |
| A-471 | CH₂—c-C₃H₅ | c-C₅H₉ |
| A-472 | CH(CH₃)—c-C₃H₅ | c-C₅H₉ |
| A-473 | CH₂—c-C₅H₉ | c-C₅H₉ |
| A-474 | CH₂—c-C₆H₁₁ | c-C₅H₉ |
| A-475 | C₆H₅ | c-C₅H₉ |
| A-476 | CH₃ | c-C₆H₁₁ |
| A-477 | C₂H₅ | c-C₆H₁₁ |
| A-478 | CH═CH₂ | c-C₆H₁₁ |
| A-479 | CH₂CH₂CH₃ | c-C₆H₁₁ |
| A-480 | CH(CH₃)₂ | c-C₆H₁₁ |
| A-481 | CH₂CH₂CH₂CH₃ | c-C₆H₁₁ |
| A-482 | C(CH₃)₃ | c-C₆H₁₁ |
| A-483 | CH₂CH(CH₃)₂ | c-C₆H₁₁ |
| A-484 | CH(CH₃)CH₂CH₃ | c-C₆H₁₁ |
| A-485 | CH₂CH═CH₂ | c-C₆H₁₁ |
| A-486 | CH₂C≡CH | c-C₆H₁₁ |
| A-487 | CH(CH₃)CH═CH₂ | c-C₆H₁₁ |
| A-488 | CHF₂ | c-C₆H₁₁ |
| A-489 | CH₂Cl | c-C₆H₁₁ |
| A-490 | CH₂CH₂CN | c-C₆H₁₁ |
| A-491 | CH₂CH₂Cl | c-C₆H₁₁ |
| A-492 | c-C₃H₅ | c-C₆H₁₁ |
| A-493 | c-C₄H₇ | c-C₆H₁₁ |
| A-494 | c-C₅H₉ | c-C₆H₁₁ |
| A-495 | c-C₆H₁₁ | c-C₆H₁₁ |
| A-496 | CH₂—c-C₃H₅ | c-C₆H₁₁ |
| A-497 | CH(CH₃)—c-C₃H₅ | c-C₆H₁₁ |
| A-498 | CH₂—c-C₅H₉ | c-C₆H₁₁ |
| A-499 | CH₂—c-C₆H₁₁ | c-C₆H₁₁ |
| A-500 | C₆H₅ | c-C₆H₁₁ |
| A-501 | CH₃ | CH₂—c-C₃H₅ |
| A-502 | C₂H₅ | CH₂—c-C₃H₅ |
| A-503 | CH═CH₂ | CH₂—c-C₃H₅ |
| A-504 | CH₂CH₂CH₃ | CH₂—c-C₃H₅ |
| A-505 | CH(CH₃)₂ | CH₂—c-C₃H₅ |
| A-506 | CH₂CH₂CH₂CH₃ | CH₂—c-C₃H₅ |
| A-507 | C(CH₃)₃ | CH₂—c-C₃H₅ |
| A-508 | CH₂CH(CH₃)₂ | CH₂—c-C₃H₅ |
| A-509 | CH(CH₃)CH₂CH₃ | CH₂—c-C₃H₅ |
| A-510 | CH₂CH═CH₂ | CH₂—c-C₃H₅ |
| A-511 | CH₂C≡CH | CH₂—c-C₃H₅ |
| A-512 | CH(CH₃)CH═CH₂ | CH₂—c-C₃H₅ |
| A-513 | CHF₂ | CH₂—c-C₃H₅ |
| A-514 | CH₂Cl | CH₂—c-C₃H₅ |
| A-515 | CH₂CH₂CN | CH₂—c-C₃H₅ |
| A-516 | CH₂CH₂Cl | CH₂—c-C₃H₅ |
| A-517 | c-C₃H₅ | CH₂—c-C₃H₅ |
| A-518 | c-C₄H₇ | CH₂—c-C₃H₅ |
| A-519 | c-C₅H₉ | CH₂—c-C₃H₅ |
| A-520 | c-C₆H₁₁ | CH₂—c-C₃H₅ |
| A-521 | CH₂—c-C₃H₅ | CH₂—c-C₃H₅ |
| A-522 | CH(CH₃)—c-C₃H₅ | CH₂—c-C₃H₅ |
| A-523 | CH₂—c-C₅H₉ | CH₂—c-C₃H₅ |
| A-524 | CH₂—c-C₆H₁₁ | CH₂—c-C₃H₅ |
| A-525 | C₆H₅ | CH₂—c-C₃H₅ |
| A-526 | CH₃ | CH(CH₃)—c-C₃H₅ |
| A-527 | C₂H₅ | CH(CH₃)—c-C₃H₅ |
| A-528 | CH═CH₂ | CH(CH₃)—c-C₃H₅ |
| A-529 | CH₂CH₂CH₃ | CH(CH₃)—c-C₃H₅ |
| A-530 | CH(CH₃)₂ | CH(CH₃)—c-C₃H₅ |
| A-531 | CH₂CH₂CH₂CH₃ | CH(CH₃)—c-C₃H₅ |
| A-532 | C(CH₃)₃ | CH(CH₃)—c-C₃H₅ |
| A-533 | CH₂CH(CH₃)₂ | CH(CH₃)—c-C₃H₅ |
| A-534 | CH(CH₃)CH₂CH₃ | CH(CH₃)—c-C₃H₅ |
| A-535 | CH₂CH═CH₂ | CH(CH₃)—c-C₃H₅ |
| A-536 | CH₂C≡CH | CH(CH₃)—c-C₃H₅ |
| A-537 | CH(CH₃)CH═CH₂ | CH(CH₃)—c-C₃H₅ |
| A-538 | CHF₂ | CH(CH₃)—c-C₃H₅ |
| A-539 | CH₂Cl | CH(CH₃)—c-C₃H₅ |
| A-540 | CH₂CH₂CN | CH(CH₃)—c-C₃H₅ |
| A-541 | CH₂CH₂Cl | CH(CH₃)—c-C₃H₅ |
| A-542 | c-C₃H₅ | CH(CH₃)—c-C₃H₅ |
| A-543 | c-C₄H₇ | CH(CH₃)—c-C₃H₅ |
| A-544 | c-C₅H₉ | CH(CH₃)—c-C₃H₅ |
| A-545 | c-C₆H₁₁ | CH(CH₃)—c-C₃H₅ |
| A-546 | CH₂—c-C₃H₅ | CH(CH₃)—c-C₃H₅ |
| A-547 | CH(CH₃)—c-C₃H₅ | CH(CH₃)—c-C₃H₅ |
| A-548 | CH₂—c-C₅H₉ | CH(CH₃)—c-C₃H₅ |
| A-549 | CH₂—c-C₆H₁₁ | CH(CH₃)—c-C₃H₅ |
| A-550 | C₆H₅ | CH(CH₃)—c-C₃H₅ |
| A-551 | CH₃ | CH₂—c-C₅H₉ |
| A-552 | C₂H₅ | CH₂—c-C₅H₉ |
| A-553 | CH═CH₂ | CH₂—c-C₅H₉ |
| A-554 | CH₂CH₂CH₃ | CH₂—c-C₅H₉ |
| A-555 | CH(CH₃)₂ | CH₂—c-C₅H₉ |
| A-556 | CH₂CH₂CH₂CH₃ | CH₂—c-C₅H₉ |
| A-557 | C(CH₃)₃ | CH₂—c-C₅H₉ |
| A-558 | CH₂CH(CH₃)₂ | CH₂—c-C₅H₉ |
| A-559 | CH(CH₃)CH₂CH₃ | CH₂—c-C₅H₉ |
| A-560 | CH₂CH═CH₂ | CH₂—c-C₅H₉ |
| A-561 | CH₂C≡CH | CH₂—c-C₅H₉ |
| A-562 | CH(CH₃)CH═CH₂ | CH₂—c-C₅H₉ |
| A-563 | CHF₂ | CH₂—c-C₅H₉ |
| A-564 | CH₂Cl | CH₂—c-C₅H₉ |
| A-565 | CH₂CH₂CN | CH₂—c-C₅H₉ |
| A-566 | CH₂CH₂Cl | CH₂—c-C₅H₉ |
| A-567 | c-C₃H₅ | CH₂—c-C₅H₉ |
| A-568 | c-C₄H₇ | CH₂—c-C₅H₉ |
| A-569 | c-C₅H₉ | CH₂—c-C₅H₉ |
| A-570 | c-C₆H₁₁ | CH₂—c-C₅H₉ |
| A-571 | CH₂—c-C₃H₅ | CH₂—c-C₅H₉ |
| A-572 | CH(CH₃)—c-C₃H₅ | CH₂—c-C₅H₉ |
| A-573 | CH₂—c-C₅H₉ | CH₂—c-C₅H₉ |
| A-574 | CH₂—c-C₆H₁₁ | CH₂—c-C₅H₉ |
| A-575 | C₆H₅ | CH₂—c-C₅H₉ |
| A-576 | CH₃ | CH₂—c-C₆H₁₁ |
| A-577 | C₂H₅ | CH₂—c-C₆H₁₁ |
| A-578 | CH═CH₂ | CH₂—c-C₆H₁₁ |
| A-579 | CH₂CH₂CH₃ | CH₂—c-C₆H₁₁ |
| A-580 | CH(CH₃)₂ | CH₂—c-C₆H₁₁ |
| A-581 | CH₂CH₂CH₂CH₃ | CH₂—c-C₆H₁₁ |
| A-582 | C(CH₃)₃ | CH₂—c-C₆H₁₁ |
| A-583 | CH₂CH(CH₃)₂ | CH₂—c-C₆H₁₁ |
| A-584 | CH(CH₃)CH₂CH₃ | CH₂—c-C₆H₁₁ |
| A-585 | CH₂CH═CH₂ | CH₂—c-C₆H₁₁ |
| A-586 | CH₂C≡CH | CH₂—c-C₆H₁₁ |
| A-587 | CH(CH₃)CH═CH₂ | CH₂—c-C₆H₁₁ |
| A-588 | CHF₂ | CH₂—c-C₆H₁₁ |
| A-589 | CH₂Cl | CH₂—c-C₆H₁₁ |
| A-590 | CH₂CH₂CN | CH₂—c-C₆H₁₁ |
| A-591 | CH₂CH₂Cl | CH₂—c-C₆H₁₁ |
| A-592 | c-C₃H₅ | CH₂—c-C₆H₁₁ |
| A-593 | c-C₄H₇ | CH₂—c-C₆H₁₁ |
| A-594 | c-C₅H₉ | CH₂—c-C₆H₁₁ |
| A-595 | c-C₆H₁₁ | CH₂—c-C₆H₁₁ |
| A-596 | CH₂—c-C₃H₅ | CH₂—c-C₆H₁₁ |
| A-597 | CH(CH₃)—c-C₃H₅ | CH₂—c-C₆H₁₁ |
| A-598 | CH₂—c-C₅H₉ | CH₂—c-C₆H₁₁ |
| A-599 | CH₂—c-C₆H₁₁ | CH₂—c-C₆H₁₁ |
| A-600 | C₆H₅ | CH₂—c-C₆H₁₁ |
| A-601 | CH₃ | C₆H₅ |
| A-602 | C₂H₅ | C₆H₅ |
| A-603 | CH═CH₂ | C₆H₅ |
| A-604 | CH₂CH₂CH₃ | C₆H₅ |
| A-605 | CH(CH₃)₂ | C₆H₅ |
| A-606 | CH₂CH₂CH₂CH₃ | C₆H₅ |
| A-607 | C(CH₃)₃ | C₆H₅ |
| A-608 | CH₂CH(CH₃)₂ | C₆H₅ |
| A-609 | CH(CH₃)CH₂CH₃ | C₆H₅ |
| A-610 | CH₂CH═CH₂ | C₆H₅ |
| A-611 | CH₂C≡CH | C₆H₅ |
| A-612 | CH(CH₃)CH═CH₂ | C₆H₅ |
| A-613 | CHF₂ | C₆H₅ |
| A-614 | CH₂Cl | C₆H₅ |
| A-615 | CH₂CH₂CN | C₆H₅ |
| A-616 | CH₂CH₂Cl | C₆H₅ |
| A-617 | c-C₃H₅ | C₆H₅ |
| A-618 | c-C₄H₇ | C₆H₅ |
| A-619 | c-C₅H₉ | C₆H₅ |
| A-620 | c-C₆H₁₁ | C₆H₅ |
| A-621 | CH₂—c-C₃H₅ | C₆H₅ |
| A-622 | CH(CH₃)—c-C₃H₅ | C₆H₅ |
| A-623 | CH₂—c-C₅H₉ | C₆H₅ |
| A-624 | CH₂—c-C₅H₉ | C₆H₅ |
| A-625 | C₆H₅ | C₆H₅ |
| A-626 | CH₃ | CH₂—c-C₄H₇ |
| A-627 | C₂H₅ | CH₂—c-C₄H₇ |
| A-628 | CH═CH₂ | CH₂—c-C₄H₇ |
| A-629 | CH₂CH₂CH₃ | CH₂—c-C₄H₇ |
| A-630 | CH(CH₃)₂ | CH₂—c-C₄H₇ |
| A-631 | CH₂CH₂CH₂CH₃ | CH₂—c-C₄H₇ |
| A-632 | C(CH₃)₃ | CH₂—c-C₄H₇ |
| A-633 | CH₂CH(CH₃)₂ | CH₂—c-C₄H₇ |
| A-634 | CH(CH₃)CH₂CH₃ | CH₂—c-C₄H₇ |
| A-635 | CH₂CH═CH₂ | CH₂—c-C₄H₇ |
| A-636 | CH₂C≡CH | CH₂—c-C₄H₇ |
| A-637 | CH(CH₃)CH═CH₂ | CH₂—c-C₄H₇ |
| A-638 | CHF₂ | CH₂—c-C₄H₇ |
| A-639 | CH₂Cl | CH₂—c-C₄H₇ |
| A-640 | CH₂CH₂CN | CH₂—c-C₄H₇ |
| A-641 | CH₂CH₂Cl | CH₂—c-C₄H₇ |
| A-642 | c-C₃H₅ | CH₂—c-C₄H₇ |
| A-643 | c-C₄H₇ | CH₂—c-C₄H₇ |
| A-644 | c-C₅H₉ | CH₂—c-C₄H₇ |
| A-645 | c-C₆H₁₁ | CH₂—c-C₄H₇ |
| A-646 | CH₂—c-C₃H₅ | CH₂—c-C₄H₇ |
| A-647 | CH(CH₃)—c-C₃H₅ | CH₂—c-C₄H₇ |
| A-648 | CH₂—c-C₅H₉ | CH₂—c-C₄H₇ |
| A-649 | CH₂—c-C₆H₁₁ | CH₂—c-C₄H₇ |
| A-650 | C₆H₅ | CH₂—c-C₄H₇ |
| A-651 | CH₃ | CH₂CH₂—c-C₃H₅ |
| A-652 | C₂H₅ | CH₂CH₂—c-C₃H₅ |
| A-653 | CH═CH₂ | CH₂CH₂—c-C₃H₅ |
| A-654 | CH₂CH₂CH₃ | CH₂CH₂—c-C₃H₅ |
| A-655 | CH(CH₃)₂ | CH₂CH₂—c-C₃H₅ |
| A-656 | CH₂CH₂CH₂CH₃ | CH₂CH₂—c-C₃H₅ |
| A-657 | C(CH₃)₃ | CH₂CH₂—c-C₃H₅ |
| A-658 | CH₂CH(CH₃)₂ | CH₂CH₂—c-C₃H₅ |
| A-659 | CH(CH₃)CH₂CH₃ | CH₂CH₂—c-C₃H₅ |
| A-660 | CH₂CH═CH₂ | CH₂CH₂—c-C₃H₅ |
| A-661 | CH₂C≡CH | CH₂CH₂—c-C₃H₅ |
| A-662 | CH(CH₃)CH═CH₂ | CH₂CH₂—c-C₃H₅ |
| A-663 | CHF₂ | CH₂CH₂—c-C₃H₅ |
| A-664 | CH₂Cl | CH₂CH₂—c-C₃H₅ |
| A-665 | CH₂CH₂CN | CH₂CH₂—c-C₃H₅ |
| A-666 | CH₂CH₂Cl | CH₂CH₂—c-C₃H₅ |
| A-667 | c-C₃H₅ | CH₂CH₂—c-C₃H₅ |
| A-668 | c-C₄H₇ | CH₂CH₂—c-C₃H₅ |
| A-669 | c-C₅H₉ | CH₂CH₂—c-C₃H₅ |
| A-670 | c-C₆H₁₁ | CH₂CH₂—c-C₃H₅ |
| A-671 | CH₂—c-C₃H₅ | CH₂CH₂—c-C₃H₅ |
| A-672 | CH(CH₃)—c-C₃H₅ | CH₂CH₂—c-C₃H₅ |
| A-673 | CH₂—c-C₅H₉ | CH₂CH₂—c-C₃H₅ |
| A-674 | CH₂—c-C₆H₁₁ | CH₂CH₂—c-C₃H₅ |
| A-675 | C₆H₅ | CH₂CH₂—c-C₃H₅ |
| A-676 | CH₃ | CH₂(CH₂)₃CH₃ |
| A-677 | C₂H₅ | CH₂(CH₂)₃CH₃ |
| A-678 | CH═CH₂ | CH₂(CH₂)₃CH₃ |
| A-679 | CH₂CH₂CH₃ | CH₂(CH₂)₃CH₃ |
| A-680 | CH(CH₃)₂ | CH₂(CH₂)₃CH₃ |
| A-681 | CH₂CH₂CH₂CH₃ | CH₂(CH₂)₃CH₃ |
| A-682 | C(CH₃)₃ | CH₂(CH₂)₃CH₃ |
| A-683 | CH₂CH(CH₃)₂ | CH₂(CH₂)₃CH₃ |
| A-684 | CH(CH₃)CH₂CH₃ | CH₂(CH₂)₃CH₃ |
| A-685 | CH₂CH═CH₂ | CH₂(CH₂)₃CH₃ |
| A-686 | CH₂C≡CH | CH₂(CH₂)₃CH₃ |
| A-687 | CH(CH₃)CH═CH₂ | CH₂(CH₂)₃CH₃ |
| A-688 | CHF₂ | CH₂(CH₂)₃CH₃ |
| A-689 | CH₂Cl | CH₂(CH₂)₃CH₃ |
| A-690 | CH₂CH₂CN | CH₂(CH₂)₃CH₃ |
| A-691 | CH₂CH₂Cl | CH₂(CH₂)₃CH₃ |
| A-692 | c-C₃H₅ | CH₂(CH₂)₃CH₃ |
| A-693 | c-C₄H₇ | CH₂(CH₂)₃CH₃ |
| A-694 | c-C₅H₉ | CH₂(CH₂)₃CH₃ |
| A-695 | c-C₆H₁₁ | CH₂(CH₂)₃CH₃ |
| A-696 | CH₂—c-C₃H₅ | CH₂(CH₂)₃CH₃ |
| A-697 | CH(CH₃)—c-C₃H₅ | CH₂(CH₂)₃CH₃ |
| A-698 | CH₂—c-C₅H₉ | CH₂(CH₂)₃CH₃ |
| A-699 | CH₂—c-C₆H₁₁ | CH₂(CH₂)₃CH₃ |
| A-700 | C₆H₅ | CH₂(CH₂)₃CH₃ |
| A-701 | CH₃ | CH(CH₃)CH(CH₃)₂ |
| A-702 | C₂H₅ | CH(CH₃)CH(CH₃)₂ |
| A-703 | CH═CH₂ | CH(CH₃)CH(CH₃)₂ |
| A-704 | CH₂CH₂CH₃ | CH(CH₃)CH(CH₃)₂ |
| A-705 | CH(CH₃)₂ | CH(CH₃)CH(CH₃)₂ |
| A-706 | CH₂CH₂CH₂CH₃ | CH(CH₃)CH(CH₃)₂ |
| A-707 | C(CH₃)₃ | CH(CH₃)CH(CH₃)₂ |
| A-708 | CH₂CH(CH₃)₂ | CH(CH₃)CH(CH₃)₂ |
| A-709 | CH(CH₃)CH₂CH₃ | CH(CH₃)CH(CH₃)₂ |
| A-710 | CH₂CH═CH₂ | CH(CH₃)CH(CH₃)₂ |
| A-711 | CH₂C≡CH | CH(CH₃)CH(CH₃)₂ |
| A-712 | CH(CH₃)CH═CH₂ | CH(CH₃)CH(CH₃)₂ |
| A-713 | CHF₂ | CH(CH₃)CH(CH₃)₂ |
| A-714 | CH₂Cl | CH(CH₃)CH(CH₃)₂ |
| A-715 | CH₂CH₂CN | CH(CH₃)CH(CH₃)₂ |
| A-716 | CH₂CH₂Cl | CH(CH₃)CH(CH₃)₂ |
| A-717 | c-C₃H₅ | CH(CH₃)CH(CH₃)₂ |
| A-718 | c-C₄H₇ | CH(CH₃)CH(CH₃)₂ |
| A-719 | c-C₅H₉ | CH(CH₃)CH(CH₃)₂ |
| A-720 | c-C₆H₁₁ | CH(CH₃)CH(CH₃)₂ |
| A-721 | CH₂—c-C₃H₅ | CH(CH₃)CH(CH₃)₂ |
| A-722 | CH(CH₃)—c-C₃H₅ | CH(CH₃)CH(CH₃)₂ |
| A-723 | CH₂—c-C₅H₉ | CH(CH₃)CH(CH₃)₂ |
| A-724 | CH₂—c-C₆H₁₁ | CH(CH₃)CH(CH₃)₂ |
| A-725 | C₆H₅ | CH(CH₃)CH(CH₃)₂ |
| A-726 | CH₃ | CH₂(CH₂)₄CH₃ |
| A-727 | C₂H₅ | CH₂(CH₂)₄CH₃ |
| A-728 | C(CH₃)₃ | CH₂(CH₂)₄CH₃ |
| A-729 | CH₂(CH₂)₄CH₃ | CH₂(CH₂)₄CH₃ |
| A-730 | CH₃ | 2-EtHex |
| A-731 | C₂H₅ | 2-EtHex |
| A-732 | C(CH₃)₃ | 2-EtHex |
| A-733 | 2-EtHex | 2-EtHex |
| A-734 | CH₃ | CH₂CH₂OH |
| A-735 | C₂H₅ | CH₂CH₂OH |
| A-736 | C(CH₃)₃ | CH₂CH₂OH |
| A-737 | CH₂CH₂CH₂CH₃ | CH₂CH₂OH |
| A-738 | CH₂(CH₂)₃CH₃ | CH₂CH₂OH |
| A-739 | CH₂CH₂OH | CH₂CH₂OH |
| A-740 | CH₂—c-C₄H₇ | CH₂—c-C₄H₇ |
| A-741 | CH₂CH₂—c-C₃H₅ | CH₂CH₂—c-C₃H₅ |
| A-742 | CH(CH₃)CH(CH₃)₂ | CH(CH₃)CH(CH₃)₂ |
| A-743 | CH₂(CH₂)₃CH₃ | CH₂(CH₂)₃CH₃ |

| | |
|---|---|
| A-744 | (CH₂)₄ |
| A-745 | CH₂CH₂SCH₂ |

c-C₃H₅: cyclopropyl; c-C₄H₇: cyclobutyl; c-C₅H₉: cyclopentyl; c-C₆H₁₁: cyclohexyl; CH₂-c-C₃H₅: cyclopropylmethyl; CH(CH₃)-c-C₃H₅: 1-cyclopropylethyl; CH₂-c-C₅H₉: cyclopentylmethyl; CH₂-c-C₅H₉: cyclopentylmethyl; C₆H₅: phenyl; CH₂CH₂-c-C₃H₅: 2-cyclopropylethyl; CH₂-c-C₄H₇: 2-cyclobutylmethyl; 2-EtHex: CH₂CH(C₂H₅)(CH₂)₃CH₃

A group of especially preferred compounds of formula I are compounds I-1 to I-40 of formula IA-1 which are listed in the table C in the example section.

In one embodiment, a compound selected from the compounds I-1 to I-40 as defined in Table C in the Example Section at the end of the description, are preferred in the methods and uses according to the invention.

In one embodiment, a compound selected from compounds I-11, I-16, I-21, I-26, I-31 is the compound I in the methods and uses according to the invention, which are defined in accordance with Table C of the example section:

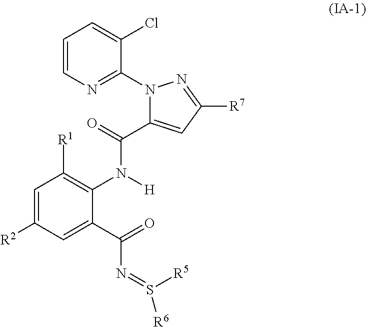

TABLE C′

| | R¹ | R² | R⁷ | R⁵ | R⁶ |
|---|---|---|---|---|---|
| I-11 | Me | Cl | CF₃ | C₂H₅ | C₂H₅ |
| I-16 | Cl | Cl | CF₃ | C₂H₅ | C₂H₅ |
| I-21 | Me | Cl | CF₃ | CH(CH₃)₂ | CH(CH₃)₂ |
| I-26 | Cl | Cl | CF₃ | CH(CH₃)₂ | CH(CH₃)₂ |
| I-31 | Br | Br | CF₃ | C₂H₅ | C₂H₅ |

In one embodiment, I-11 is the compound I in the methods and uses according to the invention.

In one embodiment, I-16 is the compound I in the methods and uses according to the invention.

In one embodiment, I-21 is the compound I in the methods and uses according to the invention.

In one embodiment, I-26 is the compound I in the methods and uses according to the invention.

In one embodiment, I-31 is the compound I in the methods and uses according to the invention.

### Compounds and Mixtures

The compounds of formula I as well as the terms “compounds for methods according to the (present) invention”, “compounds according to the (present) invention” or “compounds of formula (I)” or “compound(s) II”, which all compound(s) are applied in methods according to the present invention comprise the compound(s) as defined herein as well as a known stereoisomer, salt, tautomer or N-oxide thereof.

The term “composition(s) according to the invention” or “composition(s) of the present invention” encompasses composition(s) comprising at least one compound of formula I or mixtures of the compounds of formula I with other pesticidally active compound(s) II for being used and/or applied in methods according to the invention as defined above.

The compounds of formula I and/or their mixtures of the present invention, their stereoisomers, their salts or their N-oxides, are particularly useful for being applied in methods for soil application methods for controlling invertebrate pests, in particular for controlling insects, arthropods and nematodes and especially (spider) mites. Therefore, the invention relates to the use of compounds of formula I and/or their mixtures, for combating or controlling invertebrate pests, in particular invertebrate pests of the group of insects, arachnids or nematodes.

The present invention relates further to a composition comprising at least one compound of formula I and/or its mixtures, including a stereoisomer, salt, tautomer or N-oxide thereof, and at least one inert liquid and/or solid carrier. In particular, the invention relates to an agricultural composition comprising at least one compound of formula I and at least one liquid and/or solid carrier for use in the methods of the present invention.

Depending on the substitution pattern, the compounds of the formula (I) may have one or more centers of chirality, in which case they are present as mixtures of enantiomers or diastereomers. The invention provides both the pure enantiomers or pure diastereomers of the compounds of formula (I), and their mixtures and the use according to the invention of the pure enantiomers or pure diastereomers of the compound of formula (I) or its mixtures. Suitable compounds of the formula (I) also include all possible geometrical stereoisomers (cis/trans isomers) and mixtures thereof. Cis/trans isomers may be present with respect to an alkene, carbon-nitrogen double-bond, nitrogen-sulfur double bond or amide group. The term “stereoisomer(s)” encompasses both optical isomers, such as enantiomers or diastereomers, the latter existing due to more than one center of chirality in the molecule, as well as geometrical isomers (cis/trans isomers).

Salts of the compounds of the present invention are preferably agriculturally and veterinarily acceptable salts. They can be formed in a customary method, e.g. by reacting the compound with an acid if the compound of the present invention has a basic functionality or by reacting the compound with a suitable base if the compound of the present invention has an acidic functionality.

In general, suitable “agriculturally useful salts” or “agriculturally acceptable salts” are especially the salts of those cations or the acid addition salts of those acids whose cations and anions, respectively, do not have any adverse effect on the action of the compounds according to the present invention. Suitable cations are in particular the ions of the alkali metals, preferably lithium, sodium and potassium, of the alkaline earth metals, preferably calcium, magnesium and barium, and of the transition metals, preferably manganese, copper, zinc and iron, and also ammonium (NH₄⁺) and substituted ammonium in which one to four of the hydrogen atoms are replaced by C₁-C₄-alkyl, C₁-C₄-hydroxyalkyl, C₁-C₄-alkoxy, C₁-C₄-alkoxy-C₁-C₄-alkyl, hydroxy-C₁-C₄-alkoxy-C₁-C₄-alkyl, phenyl or benzyl. Examples of substituted ammonium ions comprise methylammonium, isopropylammonium, dimethylammonium, diisopropylammonium, trimethylammonium, tetramethylammonium, tetraethylammonium, tetrabutylammonium, 2-hydroxyethylammonium, 2-(2-hydroxyethoxyl)ethyl-ammonium, bis(2-hydroxyethyl)ammonium, benzyltrimethylammonium and benzyltriethylammonium, furthermore phosphonium ions, sulfonium ions, preferably tri(C₁-C₄-alkyl)sulfonium, and sulfoxonium ions, preferably tri(C₁-C₄-alkyl)sulfoxonium.

Anions of useful acid addition salts are primarily chloride, bromide, fluoride, hydrogen sulfate, sulfate, dihydrogen phosphate, hydrogen phosphate, phosphate, nitrate, hydrogen carbonate, carbonate, hexafluorosilicate, hexafluorophosphate, benzoate, and the anions of C₁-C₄-alkanoic acids, preferably formate, acetate, propionate and butyrate. They can be formed by reacting the compounds of the formulae I with an acid of the corresponding anion, preferably of hydrochloric acid, hydrobromic acid, sulfuric acid, phosphoric acid or nitric acid.

The compounds of the formula (I) may be present in the form of their N-oxides. The term “N-oxide” includes any compound of the present invention which has at least one tertiary nitrogen atom that is oxidized to an N-oxide moiety. N-oxides of compounds (I) can in particular be prepared by oxidizing the ring nitrogen atom(s) of the pyridine ring and/or the pyrazole ring with a suitable oxidizing agent, such as peroxo carboxylic acids or other peroxides. The person skilled in the art knows if and in which positions compounds of the formula (I) of the present invention may form N-oxides.

The compounds of the present invention may be amorphous or may exist in one ore more different crystalline states (polymorphs) which may have different macroscopic properties such as stability or show different biological properties such as activities. The present invention includes both amorphous and crystalline compounds of formula (I), their enantiomers or diastereomers, mixtures of different crystalline states of the respective compound of formula (I), its enantiomers or diastereomers, as well as amorphous or crystalline salts thereof.

The term “co-crystal” denotes a complex of the compounds according to the invention or a stereoisomer, salt, tautomer or N-oxide thereof, with one or more other molecules (preferably one molecule type), wherein usually the ratio of the compound according to the invention and the other molecule is a stoichiometric ratio.

The term “solvate” denotes a co-complex of the compounds according to the invention, or a stereoisomer, salt, tautomer or N-oxide thereof, with solvent molecules. The solvent is usually liquid. Examples of solvents are methanol, ethanol, toluol, xylol. A preferred solvent which forms solvates is water, which solvates are referred to as “hydrates”. A solvate or hydrate is usually characterized by the presence of a fixed number of n molecules solvent per m molecules compound according to the invention

The pesticidally active compounds II with which the compounds of formula I are combined with for the methods according to present invention are the following:

### Compounds (II)

In one embodiment of the invention, the compounds of formula I can be combined and used in mixture with another pesticidally active compound (II) and applied in agriculture. Preferably that other pesticidal active compound (II) is active against said soil-living arthropod pest. A skilled person is familiar with such compounds and knows which compounds are active against a specific target organism.

The compound (II) pesticides, together with which the compounds of formula I may be used according to the purpose of the present invention, and with which potential synergistic effects with regard to the method of uses might be produced, are selected and grouped according to the Mode of Action Classification from the Insecticide Resistance Action Committee (IRAC) and are

selected from group M consisting of
II-M.1 Acetylcholine esterase (AChE) inhibitors from the class of
II-M.1A carbamates, including aldicarb, alanycarb, bendiocarb, benfuracarb, butocarboxim, butoxycarboxim, carbaryl, carbofuran, carbosulfan, ethiofencarb, fenobucarb, formetanate, furathiocarb, isoprocarb, methiocarb, methomyl, metolcarb, oxamyl, pirimicarb, propoxur, thiodicarb, thiofanox, trimethacarb, XMC, xylylcarb and triazamate; or from the class of
II-M.1B organophosphates, including acephate, azamethiphos, azinphos-ethyl, azinphosmethyl, cadusafos, chlorethoxyfos, chlorfenvinphos, chlormephos, chlorpyrifos, chlorpyrifos-methyl, coumaphos, cyanophos, demeton-S-methyl, diazinon, dichlorvos/DDVP, dicrotophos, dimethoate, dimethylvinphos, disulfoton, EPN, ethion, ethoprophos, famphur, fenamiphos, fenitrothion, fenthion, fosthiazate, heptenophos, imicyafos, isofenphos, isopropyl O-(methoxyaminothio-phosphoryl) salicylate, isoxathion, malathion, mecarbam, methamidophos, methidathion, mevinphos, monocrotophos, naled, omethoate, oxydemeton-methyl, parathion, parathion-methyl, phenthoate, phorate, phosalone, phosmet, phosphamidon, phoxim, pirimiphos-methyl, profenofos, propetamphos, prothiofos, pyraclofos, pyridaphenthion, quinalphos, sulfotep, tebupirimfos, temephos, terbufos, tetrachlorvinphos, thiometon, triazophos, trichlorfon and vamidothion;
II-M.2 GABA-gated chloride channel antagonists such as:
II-M.2A cyclodiene organochlorine compounds, including endosulfan or chlordane; or
II-M.2B fiproles (phenylpyrazoles), including ethiprole, fipronil, flufiprole, pyrafluprole and pyriprole;
II-M.3 Sodium channel modulators from the class of
II-M.3A pyrethroids, including acrinathrin, allethrin, d-cis-trans allethrin, d-trans allethrin, bifenthrin, bioallethrin, bioallethrin S-cylclopentenyl, bioresmethrin, cycloprothrin, cyfluthrin, beta-cyfluthrin, cyhalothrin, lambda-cyhalothrin, gammacyhalothrin, cypermethrin, alpha-cypermethrin, beta-cypermethrin, thetacypermethrin, zeta-cypermethrin, cyphenothrin, deltamethrin, empenthrin, esfenvalerate, etofenprox, fenpropathrin, fenvalerate, flucythrinate, flumethrin, tau-fluvalinate, halfenprox, imiprothrin, meperfluthrin, metofluthrin, momfluorothrin, permethrin, phenothrin, prallethrin, profluthrin, pyrethrin (pyrethrum), resmethrin, silafluofen, tefluthrin, tetramethylfluthrin, tetramethrin, tralomethrin and transfluthrin; or
II-M.3B sodium channel modulators such as DDT or methoxychlor;
II-M.4 Nicotinic acetylcholine receptor agonists (nAChR) from the class of
II-M.4A neonicotinoids, including acetamiprid, chlothianidin, dinotefuran, imidacloprid, nitenpyram, thiacloprid and thiamethoxam; or the compounds
II-M.4A.1: 1-[(6-chloro-3-pyridinyl)methyl]-2,3,5,6,7,8-hexahydro-9-nitro-(5S,8R)-5,8-Epoxy-1H-imidazo[1,2-a]azepine; or
II-M.4A.2: 1-[(6-chloro-3-pyridyl)methyl]-2-nitro-1-[(E)-pentylideneamino]guanidine; or
II-M.4A.3: 1-[(6-chloro-3-pyridyl)methyl]-7-methyl-8-nitro-5-propoxy-3,5,6,7-tetrahydro-2H-imidazo[1,2-a]pyridine; or
II-M.4B nicotine.
II-M.5 Nicotinic acetylcholine receptor allosteric activators from the class of spinosyns, including spinosad or spinetoram;
II-M.6 Chloride channel activators from the class of avermectins and milbemycins, including abamectin, emamectin benzoate, ivermectin, lepimectin or milbemectin;
II-M.7 Juvenile hormone mimics, such as
II-M.7A juvenile hormone analogues as hydroprene, kinoprene and methoprene; or others as
II-M.7B fenoxycarb, or
II-M.7C pyriproxyfen;
II-M.8 miscellaneous non-specific (multi-site) inhibitors, including
II-M.8A alkyl halides as methyl bromide and other alkyl halides, or
II-M.8B chloropicrin, or
II-M.8C sulfuryl fluoride, or
II-M.8D borax, or
II-M.8E tartar emetic;
II-M.9 Selective homopteran feeding blockers, including
II-M.9B pymetrozine, or
II-M.9C flonicamid;
II-M.10 Mite growth inhibitors, including
II-M.10A clofentezine, hexythiazox and diflovidazin, or
II-M.10B etoxazole;
II-M.11 Microbial disruptors of insect midgut membranes, including bacillus thuringiensis or bacillus sphaericus and the insecticdal proteins they produce such as bacillus thuringiensis subsp. israelensis, bacillus sphaericus, bacillus thuringiensis subsp. aizawai, bacillus thuringiensis subsp. kurstaki and bacillus thuringiensis subsp. tenebrionis, or the Bt crop proteins: Cry1Ab, Cry1Ac, Cry1Fa, Cry2Ab, mCry3A, Cry3Ab, Cry3Bb and Cry34/35Ab1;
II-M.12 Inhibitors of mitochondrial ATP synthase, including
II-M.12A diafenthiuron, or
II-M.12B organotin miticides such as azocyclotin, cyhexatin or fenbutatin oxide, or
II-M.12C propargite, or
II-M.12D tetradifon;
II-M.13 Uncouplers of oxidative phosphorylation via disruption of the proton gradient, including chlorfenapyr, DNOC or sulfluramid;
II-M.14 Nicotinic acetylcholine receptor (nAChR) channel blockers, including nereistoxin analogues as bensultap, cartap hydrochloride, thiocyclam or thiosultap sodium;
II-M.15 Inhibitors of the chitin biosynthesis type 0, such as benzoylure including bistrifluron, chlorfluazuron, diflubenzuron, flucycloxuron, flufenoxuron, hexaflumuron, lufenuron, novaluron, noviflumuron, teflubenzuron or triflumuron;
II-M.16 Inhibitors of the chitin biosynthesis type 1, including buprofezin;
II-M.17 Moulting disruptors, Dipteran, including cyromazine;
II-M.18 Ecdyson receptor agonists such as diacylhydrazines, including methoxyfenozide, tebufenozide, halofenozide, fufenozide or chromafenozide;
II-M.19 Octopamin receptor agonists, including amitraz;
II-M.20 Mitochondrial complex III electron transport inhibitors, including
II-M.20A hydramethylnon, or
II-M.20B acequinocyl, or
II-M.20C fluacrypyrim;
II-M.21 Mitochondrial complex I electron transport inhibitors, including
II-M.21A METI acaricides and insecticides such as fenazaquin, fenpyroximate, pyrimidifen, pyridaben, tebufenpyrad or tolfenpyrad, or
II-M.21B rotenone;
II-M.22 Voltage-dependent sodium channel blockers, including
II-M.22A indoxacarb, or
II-M.22B metaflumizone; or
II-M.22C 1-[(E)-[2-(4-cyanophenyl)-1-[3-(trifluoromethyl)phenyl]ethylidene]amino]-3-[4-(difluoromethoxy)phenyl]urea;
II-M.23 Inhibitors of the acetyl CoA carboxylase, including Tetronic and Tetramic acid derivatives, including spirodiclofen, spiromesifen or spirotetramat;
II-M.24 Mitochondrial complex IV electron transport inhibitors, including
II-M.24A phosphine such as aluminium phosphide, calcium phosphide, phosphine or zinc phosphide, or
II-M.24B cyanide.
II-M.25 Mitochondrial complex II electron transport inhibitors, such as beta-ketonitrile derivatives, including cyenopyrafen or cyflumetofen;
II-M.26 Ryanodine receptor-modulators from the class of diamides, including flubendiamide, chlorantraniliprole (Rynaxypyr®), cyantraniliprole (Cyazypyr®), or the phthalamide compounds
II-M.26.1: (R)-3-Chlor-N1-{2-methyl-4-[1,2,2,2-tetrafluor-1-(trifluormethyl)ethyl]phenyl}-N2-(1-methyl-2-methylsulfonylethyl)phthalamid and
II-M.26.2: (S)-3-Chlor-N1-{2-methyl-4-[1,2,2,2-tetrafluor-1-(trifluormethyl)ethyl]phenyl}-N2-(1-methyl-2-methylsulfonylethyl)phthalamid, or the compound
II-M.26.3: 3-bromo-N-{2-bromo-4-chloro-6-[(1-cyclopropylethyl)carbamoyl]phenyl}-1-(3-chlorpyridin-2-yl)-1H-pyrazole-5-carboxamide (proposed ISO name: cyclaniliprole), or the compound
II-M.26.4: methyl-2-[3,5-dibromo-2-({[3-bromo-1-(3-chlorpyridin-2-yl)-1H-pyrazol-5-yl]carbonyl}amino)benzoyl]-1,2-dimethylhydrazinecarboxylate; or a compound selected from II-M.26.5a) to II-M.26.5d):
II-M.26.5a: N-[2-(5-amino-1,3,4-thiadiazol-2-yl)-4-chloro-6-methyl-phenyl]-5-bromo-2-(3-chloro-2-pyridyl)pyrazole-3-carboxamide;
II-M.26.5b: 5-chloro-2-(3-chloro-2-pyridyl)-N-[2,4-dichloro-6-[(1-cyano-1-methyl-ethyl)carbamoyl]phenyl]pyrazole-3-carboxamide;
II-M.26.5c: 5-bromo-N-[2,4-dichloro-6-(methylcarbamoyl)phenyl]-2-(3,5-dichloro-2-pyridyl)pyrazole-3-carboxamide;
II-M.26.5d: N-[2-(tert-butylcarbamoyl)-4-chloro-6-methyl-phenyl]-2-(3-chloro-2-pyridyl)-5-(fluoromethoxy)pyrazole-3-carboxamide; or
II-M.26.6: N2-(1-cyano-1-methyl-ethyl)-N1-(2,4-dimethylphenyl)-3-iodo-phthalamide; or
II-M.26.7: 3-chloro-N2-(1-cyano-1-methyl-ethyl)-N1-(2,4-dimethylphenyl)phthalamide;
II-M.X insecticidal active compounds of unknown or uncertain mode of action, including afidopyropen, azadirachtin, amidoflumet, benzoximate, bifenazate, bromopropylate, chinomethionat, cryolite, dicofol, flufenerim, flometoquin, fluensulfone, flupyradifurone, piperonyl butoxide, pyridalyl, pyrifluquinazon, sulfoxaflor, pyflubumide, or the compounds
II-M.X.1: 4-[5-(3,5-Dichloro-phenyl)-5-trifluoromethyl-4,5-dihydro-isoxazol-3-yl]-2-methyl-N-[(2,2,2-trifluoro-ethylcarbamoyl)-methyl]-benzamide, or the compound
II-M.X.2: cyclopropaneacetic acid, 1,1′-[(3S,4R,4aR,6S,6aS,12R,12aS,12bS)-4-[[(2-cyclopropylacetyl)oxy]methyl]-1,3,4,4a,5,6,6a,12,12a,12b-decahydro-12-hydroxy-4,6a,12b-trimethyl-11-oxo-9-(3-pyridinyl)-2H,11H-naphtho[2,1-b]pyrano[3,4-e]pyran-3,6-diyl]ester, or the compound
II-M.X.3: 11-(4-chloro-2,6-dimethylphenyl)-12-hydroxy-1,4-dioxa-9-azadispiro[4.2.4.2]-tetradec-11-en-10-one, or the compound
II-M.X.4 3-(4′-fluoro-2,4-dimethylbiphenyl-3-yl)-4-hydroxy-8-oxa-1-azaspiro[4.5]dec-3-en-2-one, or the compound
II-M.X.5: 1-[2-fluoro-4-methyl-5-[(2,2,2-trifluoroethyl)sufinyl]phenyl]-3-(trifluoromethyl)-1H-1,2,4-triazole-5-amine, or actives on basis of bacillus firmus (Votivo, I-1582), or
II-M.X.6: a compound selected from the group of
II-M.X.6a: (E/Z)—N-[1-[(6-chloro-3-pyridyl)methyl]-2-pyridylidene]-2,2,2-trifluoro-acetamide;
II-M.X.6b: (E/Z)—N-[1-[(6-chloro-5-fluoro-3-pyridyl)methyl]-2-pyridylidene]-2,2,2-trifluoro-acetamide;
II-M.X.6c: (E/Z)-2,2,2-trifluoro-N-[1-[(6-fluoro-3-pyridyl)methyl]-2-pyridylidene]acetamide;
II-M.X.6d: (E/Z)—N-[1-[(6-bromo-3-pyridyl)methyl]-2-pyridylidene]-2,2,2-trifluoro-acetamide;
II-M.X.6e: (E/Z)—N-[1-[1-(6-chloro-3-pyridyl)ethyl]-2-pyridylidene]-2,2,2-trifluoro-acetamide;
II-M.X.6f: (E/Z)—N-[1-[(6-chloro-3-pyridyl)methyl]-2-pyridylidene]-2,2-difluoro-acetamide;
II-M.X.6g: (E/Z)-2-chloro-N-[1-[(6-chloro-3-pyridyl)methyl]-2-pyridylidene]-2,2-difluoro-acetamide;
II-M.X.6h: (E/Z)—N-[1-[(2-chloropyrimidin-5-yl)methyl]-2-pyridylidene]-2,2,2-trifluoro-acetamide and II-M.X.6i: (E/Z)—N-[1-[(6-chloro-3-pyridyl)methyl]-2-pyridylidene]-2,2,3,3,3-pentafluoro-propanamide); or
II-M.X.7: triflumezopyrim; or
II-M.X.8: 4-[5-[3-chloro-5-(trifluoromethyl)phenyl]-5-(trifluoromethyl)-4H-isoxazol-3-yl]-N-[2-oxo-2-(2,2,2-trifluoroethylamino)ethyl]naphthalene-1-carboxamide, or
II-M.X.9: 3-[3-chloro-5-(trifluoromethyl)phenyl]-4-oxo-1-(pyrimidin-5-ylmethyl)pyrido[1,2-a]pyrimidin-1-ium-2-olate; or
II-M.X.10: 8-chloro-N-[2-chloro-5-methoxyphenyl)sulfonyl]-6-trifluoromethyl)-imidazo[1,2-a]pyridine-2-carboxamide; or
II-M.X.11: 4-[5-(3,5-dichlorophenyl)-5-(trifluoromethyl)-4H-isoxazol-3-yl]-2-methyl-N-(1-oxothietan-3-yl)benzamide; or
II-M.X.12: 5-[3-[2,6-dichloro-4-(3,3-dichloroallyloxyl)phenoxy]propoxy]-1H-pyrazole; or
II-M.Y Biopesticides, e.g.
II-M.Y-1: Microbial pesticides with insecticidal, acaricidal, molluscidal and/or nematicidal activity: Bacillus firmus, B. thuringiensis ssp. israelensis, B. t. ssp. galleriae, B. t. ssp. kurstaki, Beauveria bassiana, Burkholderia sp., Chromobacterium subtsugae, Cydia pomonella granulosis virus, Isaria fumosorosea, Lecanicillium longisporum, L. muscarium (formerly Verticillium lecanii), Metarhizium anisopliae, M. anisopliae var. acridum, Paecilomyces fumosoroseus, P. lilacinus, Paenibacillus poppiliae, Pasteuria spp., P. nishizawae, P. reneformis, P. usagae, Pseudomonas fluorescens, Steinernema feltiae, Streptomces galbus;
II-M.Y-2) Biochemical pesticides with insecticidal, acaricidal, molluscidal, pheromone and/or nematicidal activity: L-carvone, citral, (E,Z)-7,9-dodecadien-1-yl acetate, ethyl formate, (E,Z)-2,4-ethyl decadienoate (pear ester), (Z,Z,E)-7,11,13-hexadecatrienal, heptyl butyrate, isopropyl myristate, lavanulyl senecioate, 2-methyl 1-butanol, methyl eugenol, methyl jasmonate, (E,Z)-2,13-octadecadien-1-ol, (E,Z)-2,13-octadecadien-1-ol acetate, (E,Z)-3,13-octadecadien-1-ol, R-1-octen-3-ol, pentatermanone, potassium silicate, sorbitol actanoate, (E,Z,Z)-3,8,11-tetradecatrienyl acetate, (Z,E)-9,12-tetradecadien-1-yl acetate, Z-7-tetradecen-2-one, Z-9-tetradecen-1-yl acetate, Z-11-tetradecenal, Z-11-tetradecen-1-ol, Acacia negra extract, extract of grapefruit seeds and pulp, extract of Chenopodium ambrosiodae, Catnip oil, Neem oil, Quillay extract, Tagetes oil.

The commercially available compounds II of the group M listed above may be found in The Pesticide Manual, 15th Edition, C. D. S. Tomlin, British Crop Protection Council (2011) among other publications.

The quinoline derivative flometoquin is shown in WO2006/013896. The aminofuranone compounds flupyradifurone is known from WO 2007/115644. The sulfoximine compound sulfoxaflor is known from WO2007/149134. The pyrethroid momfluorothrin is known from U.S. Pat. No. 6,908,945. The pyrazole acaricide pyflubumide is known from WO2007/020986. The isoxazoline compound II-M.X.1 has been described in WO2005/085216, II-M.X.8 in WO2009/002809 and in WO2011/149749 and the isoxazoline II-M.X.9 in WO2013/050317. The pyripyropene derivative II-M.X.2 has been described in WO 2006/129714. The spiroketal-substituted cyclic ketoenol derivative II-M.X.3 is known from WO2006/089633 and the biphenyl-substituted spirocyclic ketoenol derivative II-M.X.4 from WO2008/067911. Triazoylphenylsulfide like II-M.X.5 have been described in WO2006/043635 and biological control agents on basis of bacillus firmus in WO2009/124707. The neonicotionids II-M4A.1 is known from WO20120/069266 and WO2011/06946, the II-M.4A.2 from WO2013/003977, the II-M4A.3. from WO2010/069266. The metaflumizone analogue II-M.22C is described in CN 10171577.

Cyantraniliprole (Cyazypyr) is known from e.g. WO 2004/067528. The phthalamides II-M.26.1 and II-M.26.2 are both known from WO 2007/101540. The anthranilamide II-M.26.3 has been described in WO 2005/077934. The hydrazide compound II-M.26.4 has been described in WO 2007/043677. The anthranilamide II-M.26.5a) is described in WO2011/085575, the II-M.26.5b) in WO2008/134969, the II-M.26.5c) in US2011/046186 and the II-M.26.5d in WO2012/034403.

The diamide compounds II-M.26.6 and II-M.26.7 can be found in CN102613183.

The compounds II-M.X.6a) to II-M.X.6i) listed in II-M.X.6 have been described in WO2012/029672.

The mesoionic antagonist compound II-M.X.8 was described in WO2012/092115, the nematicide II-M.X.9 in WO2013/055584 and the Pyridalyl-type analogue II-M.X.12 in WO2010/060379.

### Biopesticides

The biopesticides from group II-M.Y, and from group F.XIII) as described below, their preparation and their biological activity e.g. against harmful fungi, pests is known (e-Pesticide Manual V 5.2 (ISBN 978 1 901396 85 0) (2008-2011); http://www.epa.gov/opp00001/biopesticides/, see product lists therein; http://www.omri.org/omri-lists, see lists therein; Bio-Pesticides Database BPDB http://sitem.herts.ac.uk/aeru/bpdb/, see A to Z link therein). Many of these biopesticides are registered and/or are commercially available: aluminium silicate (SCREEN™ DUO from Certis LLC, USA), Ampelomyces quisqualis M-10 (e.g. AQ 10® from Intrachem Bio GmbH & Co. KG, Germany), Ascophyllum nodosum (Norwegian kelp, Brown kelp) extract (e.g. ORKA GOLD from Becker Underwood, South Africa), Aspergillus flavus NRRL 21882 (e.g. AFLAGUARD® from Syngenta, CH), Aureobasidium pullulans (e.g. BOTECTOR® from bio-ferm GmbH, Germany), Azospirillum brasilense XOH (e.g. AZOS from Xtreme Gardening, USA USA or RTI Reforestation Technologies International; USA), Bacillus amyloliquefaciens IT-45 (CNCM I 3800, NCBI 1091041) (e.g. RHIZOCELL C from ITHEC, France), B. amyloliquefaciens subsp. plantarum MBI600 (NRRL B-50595, deposited at United States Department of Agriculture) (e.g. INTEGRAL®, CLARITY, SUBTILEX NG from Becker Underwood, USA), B. pumilus QST 2808 (NRRL Accession No. B 30087) (e.g. SONATA® and BALLAD® Plus from AgraQuest Inc., USA), B. subtilis GB03 (e.g. KODIAK from Gustafson, Inc., USA), B. subtilis GB07 (EPIC from Gustafson, Inc., USA), B. subtilis QST-713 (NRRL-Nr. B 21661 in RHAPSODY®, SERENADE® MAX and SERENADE® ASO from Agra-Quest Inc., USA), B. subtilis var. amylolique [symbol] faciens FZB24 (e.g. TAEGRO® from Novozyme Biologicals, Inc., USA), B. subtilis var. amyloliquefaciens D747 (e.g. Double Nickel 55 from Certis LLC, USA), Bacillus thuringiensis ssp. kurstaki SB4 (e.g. BETA PRO® from Becker Underwood, South Africa), Beauveria bassiana GHA (BOTANIGARD® 22WGP from Laverlam Int. Corp., USA), B. bassiana 12256 (e.g. BIOEXPERT® SC from Live Systems Technology S.A., Colombia), B. bassiana PRPI 5339 (ARSEF number 5339 in the USDA ARS collection of entomopathogenic fungal cultures) (e.g. BROAD [symbol] BAND® from Becker Underwood, South Africa), Bradyrhizobium sp. (e.g. VAULT® from Becker Underwood, USA), B. japonicum (e.g. VAULT® from Becker Underwood, USA), Candida oleophila I-82 (e.g. ASPIRE® from Ecogen Inc., USA), Candida saitoana (e.g. BIO-CURE® (in mixture with lysozyme) and BIOCOAT® from Micro Flo Company, USA (BASF SE) and Arysta), Chitosan (e.g. ARMOUR-ZEN from BotriZen Ltd., NZ), Clonostachys rosea f. catenulata, also named Gliocladium catenulatum (e.g. isolate J1446: PRESTOP® from Verdera, Finland), Coniothyrium minitans CON/M/91-08 (e.g. Contans® WG from Prophyta, Germany), Cryphonectria parasitica (e.g. Endothia parasitica from CNICM, France), Cryptococcus albidus (e.g. YIELD PLUS® from Anchor Bio-Technologies, South Africa), Ecklonia maxima (kelp) extract (e.g. KELPAK SL from Kelp Products Ltd, South Africa), Fusarium oxysporum (e.g. BIOFOX® from S.I.A.P.A., Italy, FUSACLEAN® from Natural Plant Protection, France), Glomus intraradices (e.g. MYC 4000 from ITHEC, France), Glomus intraradices RTI-801 (e.g. MYKOS from Xtreme Gardening, USA or RTI Reforestation Technologies International; USA), grapefruit seeds and pulp extract (e.g. BC-1000 from Chemie S.A., Chile), Isaria fumosorosea Apopka-97 (ATCC 20874) (PFR-97™ from Certis LLC, USA), Lecanicillium muscarium (formerly Verticillium lecanii) (e.g. MYCOTAL from Koppert BV, Netherlands), Lecanicillium longisporum KV42 and KV71 (e.g. VERTALEC® from Koppert BV, Netherlands), Metarhizium anisopliae var. acridum IMI 330189 (deposited in European Culture Collections CABI) (e.g. GREEN MUSCLE® from Becker Underwood, South Africa), M. anisopliae FI-1045 (e.g. BIOCANE® from Becker Underwood Pty Ltd, Australia), M. anisopliae var. acridum FI-985 (e.g. GREEN GUARD® SC from Becker Underwood Pty Ltd, Australia), M. anisopliae F52 (e.g. MET52® Novozymes Biologicals BioAg Group, Canada), M. anisopliae ICIPE 69 (e.g. METATHRI [symbol] POL from ICIPE, Kenya), Metschnikowia fructicola (e.g. SHEMER® from Agrogreen, Israel), Microdochium dimerum (e.g. ANTIBOT® from Agrauxine, France), Neem oil (e.g. TRILOGY®, TRIACT® 70 EC from Certis LLC, USA), Paecilomyces fumosoroseus strain FE 9901 (e.g. NO FLY™ from Natural Industries, Inc., USA), P. lilacinus DSM 15169 (e.g. NEMATA® SC from Live Systems Technology S.A., Colombia), P. lilacinus BCP2 (e.g. PL GOLD from Becker Underwood BioAg SA Ltd, South Africa), mixture of Paenibacillus alvei NAS6G6 and Bacillus pumilis (e.g. BAC-UP from Becker Underwood South Africa), Penicillium bilaiae (e.g. JUMP START® from Novozymes Biologicals BioAg Group, Canada), Phlebiopsis gigantea (e.g. ROTSTOP® from Verdera, Finland), potassium silicate (e.g. Sil-MATRIX™ from Certis LLC, USA), Pseudozyma flocculosa (e.g. SPORODEX® from Plant Products Co. Ltd., Canada), Pythium oligandrum DV74 (e.g. POLYVERSUM® from Remeslo SSRO, Biopreparaty, Czech Rep.), Reynoutria sachlinensis extract (e.g. REGALIA® from Marrone BioInnovations, USA), Rhizobium leguminosarum bv. phaseolii (e.g. RHIZO-STICK from Becker Underwood, USA), R. I. trifolii (e.g. DORMAL from Becker Underwood, USA), R. I. bv. viciae (e.g. NODULATOR from Becker Underwood, USA), Sinorhizobium meliloti (e.g. DORMAL ALFALFA from Becker Underwood, USA; NITRAGIN® Gold from Novozymes Biologicals BioAg Group, Canada), Steinernema feltiae (NEMA-SHIELD® from BioWorks, Inc., USA), Streptomyces lydicus WYEC 108 (e.g. Actinovate® from Natural Industries, Inc., USA, U.S. Pat. No. 5,403,584), S. violaceusniger YCED-9 (e.g. DT-9® from Natural Industries, Inc., USA, U.S. Pat. No. 5,968,503), Talaromyces flavus V117b (e.g. PROTUS® from Prophyta, Germany), Trichoderma asperellum SKT-1 (e.g. ECO-HOPE® from Kumiai Chemical Industry Co., Ltd., Japan), T. atroviride LC52 (e.g. SENTINEL® from Agrimm Technologies Ltd, NZ), T. fertile JM41R (e.g. RICHPLUS™ from Becker Underwood Bio Ag SA Ltd, South Africa), T. harzianum T-22 (e.g. PLANTSHIELD® der Firma BioWorks Inc., USA), T. harzianum TH 35 (e.g. ROOT PRO® from Mycontrol Ltd., Israel), T. harzianum T-39 (e.g. TRICHODEX® and TRICHODERMA 2000® from Mycontrol Ltd., Israel and Makhteshim Ltd., Israel), T. harzianum and T. viride (e.g. TRICHOPEL from Agrimm Technologies Ltd, NZ), T. harzianum ICC012 and T. viride ICC080 (e.g. REMEDIER® WP from Isagro Ricerca, Italy), T. polysporum and T. harzianum (e.g. BINAB® from BINAB Bio-Innovation AB, Sweden), T. stromaticum (e.g. TRICOVAB® from C.E.P.L.A.C., Brazil), T. virens GL-21 (also named Gliocladium virens) (e.g. SOILGARD® from Certis LLC, USA), T. viride (e.g. TRIECO® from Ecosense Labs. (India) Pvt. Ltd., Indien, BIO-CURE® F from T. Stanes & Co. Ltd., Indien), T. viride TV1 (e.g. T. viride TV1 from Agribiotec srl, Italy), Ulocladium oudemansii HRU3 (e.g. BOTRY-ZEN® from Botry-Zen Ltd, NZ), Bacillus amyloliquefaciens AP-136 (NRRL B-50614), B. amyloliquefaciens AP-188 (NRRL B-50615), B. amyloliquefaciens AP-218 (NRRL B-50618), B. amyloliquefaciens AP-219 (NRRL B-50619), B. amyloliquefaciens AP-295 (NRRL B-50620), B. mojavensis AP-209 (No. NRRL B-50616), B. solisalsi AP-217 (NRRL B-50617), B. pumilus strain INR-7 (otherwise referred to as BU-F22 (NRRL B-50153) and BU-F33 (NRRL B-50185)), B. simplex ABU 288 (NRRL B-50340) and B. amyloliquefaciens subsp. plantarum MB1600 (NRRL B-50595) have been mentioned i.a. in US patent appl. 20120149571, WO 2012/079073. Beauveria bassiana DSM 12256 is known from US200020031495. Bradyrhizobium japonicum USDA is known from U.S. Pat. No. 7,262,151. Sphaerodes mycoparasitica IDAC 301008-01 (IDAC=International Depositary Authority of Canada Collection) is known from WO 2011/022809.

Bacillus amyloliquefaciens subsp. plantarum MBI600 having the accession number NRRL B-50595 is deposited with the United States Department of Agriculture on Nov. 10, 2011 under the strain designation Bacillus subtilis 1430. It has also been deposited at The National Collections of Industrial and Marine Bacteria Ltd. (NCIB), Torry Research Station, P.O. Box 31, 135 Abbey Road, Aberdeen, AB9 8DG, Scotland. under accession number 1237 on Dec. 22, 1986. Bacillus amyloliquefaciens MBI600 is known as plant growth-promoting rice seed treatment from Int. J. Microbiol. Res. ISSN 0975-5276, 3(2) (2011), 120-130 and further described e.g. in US 2012/0149571 A1. This strain MBI600 is commercially available as liquid formulation product Integral® (Becker-Underwood Inc., USA). Recently, the strain MBI 600 has been re-classified as Bacillus amyloliquefaciens subsp. plantarum based on polyphasic testing which combines classical microbiological methods relying on a mixture of traditional tools (such as culture-based methods) and molecular tools (such as genotyping and fatty acids analysis).

Thus, Bacillus subtilis MBI600 (or MBI 600 or MBI-600) is identical to Bacillus amyloliquefaciens subsp. plantarum MBI600, formerly Bacillus subtilis MBI600.

Metarhizium anisopliae IMI33 is commercially available from Becker Underwood as product Green Guard. M. anisopliae var acridium strain IMI 330189 (NRRL-50758) is commercially available from Becker Underwood as product Green Muscle.

Bacillus subtilis strain FB17 was originally isolated from red beet roots in North America (System Appl. Microbiol 27 (2004) 372-379). This Bacillus subtilis strain promotes plant health (US 2010/0260735 A1; WO 2011/109395 A2). B. subtilis FB17 has also been deposited at American Type Culture Collection (ATCC), Manassas, Va., USA, under accession number PTA-11857 on Apr. 26, 2011. Bacillus subtilis strain FB17 may also be referred to as UD1022 or UD10-22.

According to one embodiment of the inventive mixtures, the at least one biopesticide II is selected from the groups II-M.Y-1 to II-M.Y-2:
II-M.Y-1: Microbial pesticides with insecticidal, acaricidal, molluscidal and/or nematicidal activity: Bacillus firmus St 1582, B. thuringiensis ssp. israelensis SUM-6218, B. t. ssp. galleriae SDS-502, B. t. ssp. kurstaki, Beauveria bassiana GHA, B. bassiana H123, B. bassiana DSM 12256, B. bassiana PRPI 5339, Burkholderia sp. A396, Chromobacterium subtsugae PRAA4-1T, Cydia pomonella granulosis virus isolate V22, Isaria fumosorosea Apopka-97, Lecanicillium longisporum KV42, L. longisporum KV71, L. muscarium (formerly Verticillium lecanii), Metarhizium anisopliae FI-985, M. anisopliae FI-1045, M. anisopliae F52, M. anisopliae ICIPE 69, M. anisopliae var. acridum IMI 330189, Paecilomyces fumosoroseus FE 9901, P. lilacinus DSM 15169, P. lilacinus BCP2, Paenibacillus poppiliae Dutky-1940 (NRRL B-2309=ATCC 14706), P. poppiliae KLN 3, P. poppiliae Dutky 1, Pasteuria spp. Ph3, P. nishizawae PN-1, P. reneformis Pr-3, P. usagae, Pseudomonas fluorescens CL 145A, Steinernema feltiae, Streptomces galbus;
II-M.Y-2: Biochemical pesticides with insecticidal, acaricidal, molluscidal, pheromone and/or nematicidal activity: L-carvone, citral, (E,Z)-7,9-dodecadien-1-yl acetate, ethyl formate, (E,Z)-2,4-ethyl decadienoate (pear ester), (Z,Z,E)-7,11,13-hexadecatrienal, heptyl butyrate, isopropyl myristate, lavanulyl senecioate, 2-methyl 1-butanol, methyl eugenol, methyl jasmonate, (E,Z)-2,13-octadecadien-1-ol, (E,Z)-2,13-octadecadien-1-ol acetate, (E,Z)-3,13-octadecadien-1-ol, R-1-octen-3-ol, pentatermanone, potassium silicate, sorbitol actanoate, (E,Z,Z)-3,8,11-tetradecatrienyl acetate, (Z,E)-9,12-tetradecadien-1-yl acetate, Z-7-tetradecen-2-one, Z-9-tetradecen-1-yl acetate, Z-11-tetradecenal, Z-11-tetradecen-1-ol, Acacia negra extract, extract of grapefruit seeds and pulp, extract of Chenopodium ambrosiodae, Catnip oil, Neem oil, Quillay extract, Tagetes oil;

According to one embodiment of the inventive mixtures, the at least one biopesticide II is selected from group II-M.Y-1.

According to one embodiment of the inventive mixtures, the at least one biopesticide II is selected from II-M.Y-2.

According to one embodiment of the inventive mixtures, the at least one biopesticide II is Bacillus amyloliquefaciens subsp. plantarum MBI600. These mixtures are particularly suitable in soybean.

According to another embodiment of the inventive mixtures, the at least one biopesticide II is B. pumilus strain INR-7 (otherwise referred to as BU-F22 (NRRL B-50153) and BU-F33 (NRRL B-50185; see WO 2012/079073). These mixtures are particularly suitable in soybean and corn.

According to another embodiment of the inventive mixtures, the at least one biopesticide II is Bacillus pumilus, preferably B. pumilis strain INR-7 (otherwise referred to as BU-F22 (NRRL B-50153) and BU-F33 (NRRL B-50185). These mixtures are particularly suitable in soybean and corn.

According to another embodiment of the inventive mixtures, the at least one biopesticide II is Bacillus simplex, preferably B. simplex strain ABU 288 (NRRL B-50340). These mixtures are particularly suitable in soybean and corn.

According to another embodiment of the inventive mixtures, the at least one biopesticide II is selected from Trichoderma asperellum, T. atroviride, T. fertile, T. gamsii, T. harmatum; mixture of T. harzia [symbol] num and T. viride; mixture of T. polysporum and T. harzianum; T. stromaticum, T. virens (also named Gliocladium virens) and T. viride; preferably Trichoderma fertile, in particular T. fertile strain JM41R. These mixtures are particularly suitable in soybean and corn.

According to another embodiment of the inventive mixtures, the at least one biopesticide II is Sphaerodes mycoparasitica, preferably Sphaerodes mycoparasitica strain IDAC 301008-01 (also referred to as strain SMCD2220-01). These mixtures are particularly suitable in soybean and corn.

According to another embodiment of the inventive mixtures, the at least one biopesticide II is Beauveria bassiana, preferably Beauveria bassiana strain PPRI5339. These mixtures are particularly suitable in soybean and corn.

According to another embodiment of the inventive mixtures, the at least one biopesticide II is Metarhizium anisopliae or M. anisopliae var. acridium, preferably selected from M anisolpiae strain IMI33 and M. anisopliae var. acridium strain IMI 330189. These mixtures are particularly suitable in soybean and corn.

According to another embodiment of the inventive mixtures, Bradyrhizobium sp. (meaning any Bradyrhizobium species and/or strain) as biopesticide II is Bradyrhizobium japonicum (B. japonicum). These mixtures are particularly suitable in soybean. Preferably B. japonicum is not one of the strains TA-11 or 532c. B. japonicum strains were cultivated using media and fermentation techniques known in the art, e.g. in yeast extract-mannitol broth (YEM) at 27° C. for about 5 days.

References for various B. japonicum strains are given e.g. in U.S. Pat. No. 7,262,151 (B. japonicum strains USDA 110 (=IITA 2121, SEMIA 5032, RCR 3427, ARS 1-110, Nitragin 61A89; isolated from Glycine max in Florida in 1959, Serogroup 110; Appl Environ Microbiol 60, 940-94, 1994), USDA 31 (=Nitragin 61A164; isolated from Glycine max in Wisconsin in 1941, USA, Serogroup 31), USDA 76 (plant passage of strain USDA 74 which has been isolated from Glycine max in California, USA, in 1956, Serogroup 76), USDA 121 (isolated from Glycine max in Ohio, USA, in 1965), USDA 3 (isolated from Glycine max in Virginia, USA, in 1914, Serogroup 6) and USDA 136 (=CB 1809, SEMIA 586, Nitragin 61A136, RCR 3407; isolated from Glycine max in Beltsville, Md. in 1961; Appl Environ Microbiol 60, 940-94, 1994). USDA refers to United States Department of Agriculture Culture Collection, Beltsville, Md., USA (see e.g. Beltsville Rhizobium Culture Collection Catalog March 1987 ARS-30). Further suitable B. japonicum strain G49 (INRA, Angers, France) is described in Fernandez-Flouret, D. & Cleyet-Marel, J. C. (1987) C R Acad Agric Fr 73, 163-171), especially for soybean grown in Europe, in particular in France. Further suitable B. japonicum strain TA-11 (TA11 NOD+) (NRRL B-18466) is i.a. described in U.S. Pat. No. 5,021,076; Appl Environ Microbiol (1990) 56, 2399-2403 and commercially available as liquid inoculant for soybean (VAULT® NP, Becker Underwood, USA). Further B. japonicum strains as example for biopesticide II are described in US2012/0252672A. Further suitable and especially in Canada commercially available strain 532c (The Nitragin Company, Milwaukee, Wis., USA, field isolate from Wisconsin; Nitragin strain collection No. 61A152; Can J Plant Sci 70 (1990), 661-666).

Other suitable and commercially available B. japonicum strains (see e.g. Appl Environ Microbiol 2007, 73(8), 2635) are SEMIA 566 (isolated from North American inoculant in 1966 and used in Brazilian commercial inoculants from 1966 to 1978), SEMIA 586 (=CB 1809; originally isolated in Maryland, USA but received from Australia in 1966 and used in Brazilian inoculants in 1977), CPAC 15 (=SEMIA 5079; a natural variant of SEMIA 566 used in commercial inoculants since 1992) and CPAC 7 (=SEMIA 5080; a natural variant of SEMIA 586 used in commercial inoculants since 1992). These strains are especially suitable for soybean grown in Australia or South America, in particular in Brazil. Some of the abovementioned strains have been re-classified as a novel species Bradyrhizobium elkanii, e.g. strain USDA 76 (Can. J. Microbiol., 1992, 38, 501-505).

Another suitable and commercially available B. japonicum strain is E-109 (variant of strain USDA 138, see e.g. Eur. J. Soil Biol. 45 (2009) 28-35; Biol Fertil Soils (2011) 47:81-89, deposited at Agriculture Collection Laboratory of the Instituto de Microbiologia y Zoologia Agricola (IMYZA), Instituto Nacional de Tecnologi'a Agropecuaria (INTA), Castelar, Argentina). This strain is especially suitable for soybean grown in South America, in particular in Argentina.

The present invention also relates to mixtures, wherein the at least one biopesticide II is selected from Bradyrhizobium elkanii and Bradyrhizobium liaoningense (B. elkanii and B. liaoningense), more preferably from B. elkanii. These mixtures are particularly suitable in soybean. B. elkanii and liaoningense were cultivated using media and fermentation techniques known in the art, e.g. in yeast extract-mannitol broth (YEM) at 27° C. for about 5 days.

Suitable and commercially available B. elkanii strains are SEMIA 587 and SEMIA 5019 (=29W) (see e.g. Appl Environ Microbiol 2007, 73(8), 2635) and USDA 3254 and USDA 76 and USDA 94. Further commercially available B. elkanii strains are U-1301 and U-1302 (e.g. product Nitroagin® Optimize from Novozymes Bio As S.A., Brazil or NITRASEC for soybean from LAGE y Cia, Brazil). These strains are especially suitable for soybean grown in Australia or South America, in particular in Brazil.

The present invention also relates to mixtures, wherein the at least one biopesticide II is selected from Bradyrhizobium japonicum (B. japonicum) and further comprises a compound III, wherein compound III is selected from jasmonic acid or salts or derivatives thereof including cis-jasmone, preferably methyl-jasmonate or cis-jasmone.

The present invention also relates to mixtures, wherein biopesticide II is selected from Bradyrhizobium sp. (Arachis) (B. sp. Arachis) which shall describe the cowpea miscellany cross-inoculation group which includes inter alia indigenous cowpea bradyrhizobia on cowpea (Vigna unguiculata), siratro (Macroptilium atropurpureum), lima bean (Phaseolus lunatus), and peanut (Arachis hypogaea). This mixture comprising as biopesticide II B. sp. Arachis is especially suitable for use in peanut, Cowpea, Mung bean, Moth bean, Dune bean, Rice bean, Snake bean and Creeping vigna, in particular peanut.

Suitable and commercially available B. sp. (Arachis) strain is CB1015 (=IITA 1006, USDA 3446 presumably originally collected in India; from Australian Inoculants Research Group; see e.g. http://www.qaseeds.com.au/inoculant_applic.php; Beltsville Rhizobium Culture Collection Catalog March 1987 USDA-ARS ARS-30). These strains are especially suitable for peanut grown in Australia, North America or South America, in particular in Brazil. Further suitable strain is bradyrhizobium sp. PNL01 (Becker Underwood; ISO Rep Marita McCreary, QC Manager Padma Somasageran; IDENTIFICATION OF RHIZOBIA SPECIES THAT CAN ESTABLISH NITROGEN-FIXING NODULES IN CROTALARIA LONGIROSTRATA. Apr. 29, 2010, University of Massachusetts Amherst: http://www.wpi.edu/Pubs/E-project/Available/E-project-042810-163614/unrestricted/Bisson.Mason._Identification_of_Rhizobia_Species_That_can_Establish_Nitrogen-Fixing_Nodules_in_Crotalia_Longirostrata.pdf).

Suitable and commercially available Bradyrhizobium sp. (Arachis) strains especially for cowpea and peanut but also for soybean are Bradyrhizobium SEMIA 6144, SEMIA 6462 (=BR 3267) and SEMIA 6464 (=BR 3262) (deposited at FEPAGRO-MIRCEN, R. Gonçalves Dias, 570 Porto Alegre—RS, 90130-060, Brazil; see e.g. FEMS Microbiology Letters (2010) 303(2), 123-131; Revista Brasileira de Ciencia do Solo (2011) 35(3); 739-742, ISSN 0100-0683).

The present invention also relates to mixtures wherein the at least one biopesticide II is selected from Bradyrhizobium sp. (Arachis) and further comprises a compound III, wherein compound III is selected from jasmonic acid or salts or derivatives thereof including cis-jasmone, preferably methyl-jasmonate or cis-jasmone.

The present invention also relates to mixtures, wherein the at least one biopesticide II is selected from Bradyrhizobium sp. (Lupine) (also called B. lupini, B. lupines or Rhizobium lupini). This mixture is especially suitable for use in dry beans and lupins.

Suitable and commercially available B. lupini strain is LL13 (isolated from Lupinus iuteus nodules from French soils; deposited at INRA, Dijon and Angers, France; http://agriculture.gouv.fr/IMG/pdf/ch20060216.pdf). This strain is especially suitable for lupins grown in Australia, North America or Europe, in particular in Europe.

Further suitable and commercially available B. lupini strains WU425 (isolated in Esperance, Western Australia from a non-Australian legume Ornthopus compressus), WSM4024 (isolated from lupins in Australia by CRS during a 2005 survey) and WSM471 (isolated from Ornithopus pinnatus in Oyster Harbour, Western Australia) are described e.g. in Palta J. A. and Berger J. B. (eds), 2008, Proceedings 12th International Lupin Conference, 14-18 Sep. 2008, Fremantle, Western Australia. International Lupin Association, Canterbury, New Zealand, 47-50, ISBN 0-86476-153-8: http://www.lupins.org/pdf/conference/2008/Agronomy%20and%20Production/John%20Howieson%20and%20G%20OHara.pdf; Appl Environ Microbiol (2005) 71, 7041-7052 and Australian J. Exp. Agricult. (1996) 36(1), 63-70.

The present invention also relates to mixtures wherein the at least one biopesticide II is selected from Bradyrhizobium sp. (Lupine) (B. lupini) and further comprises a compound III, wherein compound III is selected from jasmonic acid or salts or derivatives thereof including cis-jasmone, preferably methyl-jasmonate or cis-jasmone.

The present invention also relates to mixtures, wherein the at least one biopesticide II is selected from Mesorhizobium sp. (meaning any Mesorhizobium species and/or strain), more preferably Mesorhizobium ciceri. These mixtures are particularly suitable in cowpea.

Suitable and commercially available M. sp. strains are e.g. M. ciceri CC1192 (=UPM 848, CECT 5549; from Horticultural Research Station, Gosford, Australia; collected in Israel from Cicer arietinum nodules; Can J Microbial (2002) 48, 279-284) and Mesorhizobium sp. strains WSM1271 (collected in Sardinia, Italy, from plant host Biserrula pelecinus), WSM 1497 (collected in Mykonos, Greece, from plant host Biserrula pelecinus), M. loti strains CC829 (commercial inoculant for Lotus pedunculatus and L. ulginosus in Australia, isolated from L. ulginosus nodules in USA) and SU343 (commercial inoculant for Lotus corniculatus in Australia; isolated from host nodules in USA) all of which are deposited at Western Australian Soil Microbiology (WSM) culture collection, Australia and/or CSIRO collection (CC), Canberra, Australian Capital Territory (see e.g. Soil Biol Biochem (2004) 36(8), 1309-1317; Plant and Soil (2011) 348(1-2), 231-243). Suitable and commercially available M. loti strains are e.g. M. loti CC829 for Lotus pedunculatus.

The present invention also relates to mixtures wherein the at least one biopesticide II is selected from Bradyrhizobium sp. (Lupine) (B. lupini) and further comprises a compound III, wherein compound III is selected from jasmonic acid or salts or derivatives thereof including cis-jasmone, preferably methyl-jasmonate or cis-jasmone.

The present invention also relates to mixtures wherein the at least one biopesticide II is selected from Mesorhizobium huakuii, also referred to as Rhizobium huakuii (see e.g. Appl. Environ. Microbiol. 2011, 77(15), 5513-5516). These mixtures are particularly suitable in Astralagus, e.g. Astalagus sinicus (Chinese milkwetch), Thermopsis, e.g. Thermopsis luinoides (Goldenbanner) and alike.

Suitable and commercially available M. huakuii strain is HN3015 which was isolated from Astralagus sinicus in a rice-growing field of Southern China (see e.g. World J. Microbiol. Biotechn. (2007) 23(6), 845-851, ISSN 0959-3993).

The present invention also relates to mixtures wherein the at least one biopesticide II is selected from Mesorhizobium huakuii and further comprises a compound III, wherein compound III is selected from jasmonic acid or salts or derivatives thereof including cis-jasmone, preferably methyl-jasmonate or cis-jasmone.

The present invention also relates to mixtures, wherein the at least one biopesticide II is selected from Azospirillum amazonense, A. brasilense, A. lipoferum, A. irakense, A. halopraeferens, more preferably from A. brasilense, in particular selected from A. brasilense strains BR 11005 (SP 245) and AZ39 which are both commercially used in Brazil and are obtainable from EM-BRAPA, Brazil. These mixtures are particularly suitable in soybean.

Humates are humic and fulvic acids extracted from a form of lignite coal and clay, known as leonardite. Humic acids are organic acids that occur in humus and other organically derived materials such as peat and certain soft coal. They have been shown to increase fertilizer efficiency in phosphate and micro-nutrient uptake by plants as well as aiding in the development of plant root systems.

Salts of jasmonic acid (jasmonate) or derivatives include without limitation the jasmonate salts potassium jasmonate, sodium jasmonate, lithium jasmonate, ammonium jasmonate, dimethylammonium jasmonate, isopropylammonium jasmonate, diolammonium jasmonate, diethtriethanolammonium jasmonate, jasmonic acid methyl ester, jasmonic acid amide, jasmonic acid methylamide, jasmonic acid-L-amino acid (amide-linked) conjugates (e.g., conjugates with L-isoleucine, L-valine, L-leucine, or L-phenylalanine), 12-oxo-phytodienoic acid, coronatine, coronafacoyl-L-serine, coronafacoyl-L-threonine, methyl esters of 1-oxo-indanoyl-isoleucine, methyl esters of 1-oxo-indanoyl-leucine, coronalon (2-[(6-ethyl-1-oxo-indane-4-carbonyl)-amino]-3-methyl-pentanoic acid methyl ester), linoleic acid or derivatives thereof and cis-jasmone, or combinations of any of the above.

According to one embodiment, the microbial pesticides embrace not only the isolated, pure cultures of the respective micro-organism as defined herein, but also its cell-free extract, its suspensions in a whole broth culture or as a metabolite-containing supernatant or a purified metabolite obtained from a whole broth culture of the microorganism or microorganism strain.

According to a further embodiment, the microbial pesticides embrace not only the isolated, pure cultures of the respective micro-organism as defined herein, but also a cell-free extract thereof or at least one metabolite thereof, and/or a mutant of the respective micro-organism having all the identifying characteristics thereof and also a cell-free extract or at least one metabolite of the mutant.

“Whole broth culture” refers to a liquid culture containing both cells and media.

“Supernatant” refers to the liquid broth remaining when cells grown in broth are removed by centrifugation, filtration, sedimentation, or other means well known in the art.

The term “metabolite” refers to any compound, substance or byproduct produced by a microorganism (such as fungi and bacteria) that has improves plant growth, water use efficiency of the plant, plant health, plant appearance, or the population of beneficial microorganisms in the soil around the plant activity.

The term “mutant” refers a microorganism obtained by direct mutant selection but also includes microorganisms that have been further mutagenized or otherwise manipulated (e.g., via the introduction of a plasmid). Accordingly, embodiments include mutants, variants, and or derivatives of the respective microorganism, both naturally occurring and artificially induced mutants. For example, mutants may be induced by subjecting the microorganism to known mutagens, such as N-methyl-nitrosoguanidine, using conventional methods.

According to the invention, the solid material (dry matter) of the biopesticides (with the exception of oils such as Neem oil, Tagetes oil, etc.) are considered as active components (e.g. to be obtained after drying or evaporation of the extraction medium or the suspension medium in case of liquid formulations of the microbial pesticides).

In accordance with the present invention, the weight ratios and percentages used herein for biological extract such as Quillay extract are based on the total weight of the dry content (solid material) of the respective extract(s).

For microbial pesticides, weight ratios and/or percentages refer to the total weight of a preparation of the respective biopesticide with at least 1×106 CFU/g (“colony forming units per gram total weight”), preferably with at least 1×108 CFU/g, even more preferably from 1×108 to 1×1012 CFU/g dry matter. Colony forming unit is measure of viable microbial cells, in particular fungal and bacterial cells. In addition, here CFU may also be understood as number of (juvenile) individual nematodes in case of (entomo-pathogenic) nematode biopesticides, such as Steinernema feltiae.

Herein, microbial pesticides may be supplied in any physiological state such as active or dormant. Such dormant active component may be supplied for example frozen, dried, or lyophilized or partly desiccated (procedures to produce these partly desiccated organisms are given in WO2008/002371) or in form of spores.

Microbial pesticides used as organism in an active state can be delivered in a growth medium without any additional additives or materials or in combination with suitable nutrient mixtures. According to a further embodiment, microbial pesticides are delivered and formulated in a dormant stage, more preferably in form of spores.

The total weight ratios of compositions, which comprise a microbial pesticide as component 2, can be determined based on the total weight of the solid material (dry matter) of component 1) and using the amount of CFU of component 2) to calculate the total weight of component 2) with the following equation that 1×10⁹ CFU equals one gram of total weight of component 2). According to one embodiment, the compositions, which comprise a microbial pesticide, comprise between 0.01 and 90% (w/w) of dry matter (solid material) of component 1) and from 1×10⁵ CFU to 1×10¹² CFU of component 2) per gram total weight of the composition.

According to another embodiment, the compositions, which comprise a microbial pesticide, comprise between 5 and 70% (w/w) of dry matter (solid material) of component 1) and from 1×10⁶ CFU to 1×10¹⁰ CFU of component 2) per gram total weight of the composition.

According to another embodiment, the compositions, wherein one component is a microbial pesticide, comprise between 25 and 70% (w/w) of dry matter (solid material) of component 1) and from 1×10⁷ CFU to 1×10⁹ CFU of component 2) per gram total weight of the composition. In the case of mixtures comprising a microbial pesticide, the application rates preferably range from about 1×10⁶ to 5×10¹⁵ (or more) CFU/ha. Preferably, the spore concentration is about 1×107 to about 1×1011 CFU/ha. In the case of (entomopathogenic) nematodes as microbial pesticides (e.g. Steinernema feltiae), the application rates preferably range inform about 1×10⁵ to 1×10¹² (or more), more preferably from 1×10⁸ to 1×10¹¹, even more preferably from 5×10⁸ to 1×10¹⁰ individuals (e.g. in the form of eggs, juvenile or any other live stages, preferably in an infective juvenile stage) per ha.

In the case of mixtures comprising microbial pesticides, the application rates with respect to plant propagation material preferably range from about 1×10⁶ to 1×10¹² (or more) CFU/seed. Preferably, the concentration is about 1×10⁶ to about 1×10¹¹ CFU/seed. In the case of microbial pesticides, the application rates with respect to plant propagation material also preferably range from about 1×10⁷ to 1×10¹⁴ (or more) CFU per 100 kg of seed, preferably from 1×10⁹ to about 1×10¹¹ CFU per 100 kg of seed.

In another embodiment of the invention, the compound (II) pesticides, together with which the compounds of formula I may be used according to the purpose of the present invention, and with which potential synergistic effects with regard to the method of uses might be produced, are selected from from group F consisting of
F.I) Respiration Inhibitors
F.I-1) Inhibitors of complex III at Qo site selected from the group of strobilurins including azoxystrobin, coumethoxystrobin, coumoxystrobin, dimoxystrobin, enestroburin, fluoxastrobin, kresoxim-methyl, mandestrobin, metominostrobin, orysastrobin, picoxystrobin, pyraclostrobin, pyrametostrobin, pyraoxystrobin, pyribencarb, triclopyricarb/chlorodincarb, trifloxystrobin, 2-[2-(2,5-dimethylphenoxymethyl)-phenyl]-3-methoxy-acrylic acid methyl ester and 2 (2-(3-(2,6-dichlorophenyl)-1-methyl-allylideneaminooxymethyl)-phenyl)-2-methoxyimino-N methyl-acetamide;
   oxazolidinediones and imidazolinones selected from famoxadone, fenamidone;
F.I-2) Inhibitors of complex II selected from the group of carboxamides, including carboxanilides selected from benodanil, benzovindiflupyr, bixafen, boscalid, carboxin, fenfuram, fenhexamid, fluopyram, flutolanil, furametpyr, isofetamid, isopyrazam, isotianil, mepronil, oxycarboxin, penflufen, penthiopyrad, sedaxane, tecloftalam, thifluzamide, tiadinil, 2-amino-4 methyl-thiazole-5-carboxanilide, N-(3′,4′,5′ trifluorobiphenyl-2 yl)-3-difluoromethyl-1-methyl-1H-pyrazole-4 carboxamide (fluxapyroxad), N-(4′-trifluoromethylthiobiphenyl-2-yl)-3 difluoromethyl-1-methyl-1H pyrazole-4-carboxamide, N-(2-(1,3,3-trimethylbutyl)-phenyl)-1,3-dimethyl-5 fluoro-1H-pyrazole-4 carboxamide, 3 (difluoromethyl)-1-methyl-N-(1,1,3-trimethyl [symbol] indan-4-yl)pyrazole-4-carboxamide, 3 (trifluoromethyl)-1-methyl-N-(1,1,3-trimethyl [symbol] indan-4-yl)pyrazole-4-carboxamide, 1,3-dimethyl-N-(1,1,3-trimethylindan-4-yl)pyr [symbol] azole-4-carboxamide, 3-(trifluorometh [symbol] yl)-1,5-dimethyl-N-(1,1,3-trimethylindan-4-yl) [symbol] pyrazole-4-carboxamide, 3-(difluoro [symbol] methyl)-1,5-dimethyl-N-(1,1,3-trimethylindan-4 yl)pyrazole-4-carboxamide, 3-(trifluorometh [symbol] yl)-1,5-dimethyl-N-(1,1,3-trimethylindan-4-yl) [symbol] pyrazole-4-carboxamide, 1,3,5-tri [symbol] methyl-N-(1,1,3-trimethylindan-4-yl)pyrazole-4-carboxamide N-(7-fluoro-1,1,3-trimethyl-indan-4-yl)-1,3-dimethyl-pyrazole-4-carbox [symbol] amide, N-[2-(2,4-dichlorophenyl)-2-methoxy-1-methyl-ethyl]-3-(difluoromethyl)-1-methyl-pyrazole-4-carboxamide;
F.I-3) Inhibitors of complex III at Qi site including cyazofamid, amisulbrom, [(3S,6S,7R,8R)-8-benzyl-3-[(3-acetoxy-4-methoxy-pyridine-2-carbonyl)amino]-6-methyl-4,9-dioxo-1,5-dioxonan-7-yl]2-methylpropanoate, [(3S,6S,7R,8R)-8-benzyl-3-[[3-(acetoxymethoxy)-4-methoxy-pyridine-2-carbonyl]amino]-6-methyl-4,9-dioxo-1,5-dioxonan-7-yl]2-methylpropanoate, [(3S,6S,7R,8R)-8-benzyl-3-[(3-isobutoxycarbonyloxy-4-methoxy-pyridine-2-carbonyl)amino]-6-methyl-4,9-dioxo-1,5-dioxonan-7-yl]2-methylpropanoate, [(3S,6S,7R,8R)-8-benzyl-3-[[3-(1,3-benzodioxol-5-ylmethoxy)-4-methoxy-pyridine-2-carbonyl]amino]-6-methyl-4,9-dioxo-1,5-dioxonan-7-yl]2-methylpropanoate, 3S,6S,7R,8R)-3-[[(3-hydroxy-4-methoxy-2-pyridinyl)carbonyl]amino]-6-methyl-4,9-dioxo-8-(phenylmethyl)-1,5-dioxonan-7-yl 2-methylpropanoate;
F.I-4) Other respiration inhibitors (complex I uncouplers), including diflumetorim; (5,8-difluoroquinazolin-4-yl)-{2-[2-fluoro-4-(4-trifluoromethylpyridin-2-yloxy)phenyl]-ethyl}-amine; tecnazen; ametoctradin; silthiofam;
   and including nitrophenyl derivates selected from binapacryl, dinobuton, dinocap, fluazinam, ferimzone; nitrthal-isopropyl,
   and including organometal compounds selected from fentin salts, including fentin-acetate, fentin chloride or fentin hydroxide;
F.II) Sterol biosynthesis inhibitors (SBI fungicides)
F.II-1) C14 demethylase inhibitors,
   including triazoles selected from azaconazole, bitertanol, bromuconazole, cyproconazole, difenoconazole, diniconazole, diniconazole-M, epoxiconazole, fenbuconazole, fluquinconazole, flusilazole, flutriafol, hexaconazole, imibenconazole, ipconazole, metconazole, myclobutanil, paclobutrazole, penconazole, propiconazole, prothioconazole, simeconazole, tebuconazole, tetraconazole, triadimefon, triadimenol, triticonazole, uniconazole, 1-[rel-(2S;3R)-3-(2-chlorophenyl)-2-(2,4-difluorophenyl)-oxiranylmethyl]-5-thiocyanato-1H[1,2,4]triazole, 2-[rel-(2S; 3R)-3-(2-chlorophenyl)-2-(2,4-difluorophenyl)oxiranylmethyl]-2H-[1,2,4]triazole-3-thiol, 2-[2-chloro-4-(4-chlorophenoxyl)phenyl]-1-(1,2,4-triazol-1-yl)pentan-2-ol, 1-[4-(4-chlorophenoxy)-2-(trifluoromethyl)phenyl]-1-cyclopropyl-2-(1,2,4-triazol-1-yl)ethanol, 2-[4-(4-chlorophenoxy)-2-(trifluoromethyl)phenyl]-1-(1,2,4-triazol-1-yl)butan-2-ol, 2-[2-chloro-4-(4-chlorophenoxyl)phenyl]-1-(1,2,4-triazol-1-yl)butan-2-ol, 2-[4-(4-chlorophenoxy)-2-(trifluoromethyl)phenyl]-3-methyl-1-(1,2,4-triazol-1-yl)butan-2-ol, 2-[4-(4-chlorophenoxy)-2-(trifluoromethyl)phenyl]-1-(1,2,4-triazol-1-yl)propan-2-ol, 2-[2-chloro-4-(4-chlorophenoxyl)phenyl]-3-methyl-1-(1,2,4-triazol-1-yl)butan-2-ol, 2-[4-(4-chlorophenoxy)-2-(trifluoromethyl)phenyl]-1-(1,2,4-triazol-1-yl)pentan-2-ol, 2-[4-(4-fluorophenoxy)-2-(trifluoromethyl)phenyl]-1-(1,2,4-triazol-1-yl)propan-2-ol; and including imidazoles selected from imazalil, pefurazoate, oxpoconazole, prochloraz, triflumizole;
   and including pyrimidines, pyridines and piperazines selected from fenarimol, nuarimol, pyrifenox, triforine, [3-(4-chloro-2-fluoro-phenyl)-5-(2,4-difluorophenyl)isoxazol-4-yl]-(3-pyridyl)methanol;
F.II-2) Delta14-reductase inhitors,
   including morpholines selected from aldimorph, dodemorph, dodemorphacetate, fenpropimorph, tridemorph;
   and including piperidines selected from fenpropidin, piperalin;
   and including spiroketalamines selected from spiroxamine;
F.II-3) Inhibitors of 3-keto reductase including hydroxyanilides selected from fenhexamid;
F.III) Nucleic acid synthesis inhibitors
F.III-1) RNA, DNA synthesis inhibitors,
   including phenylamides or acyl amino acid fungicides selected from benalaxyl, benalaxyl-M, kiralaxyl, metalaxyl, metalaxyl-M (mefenoxam), ofurace, oxadixyl;
   and including isoxazoles and iosothiazolones selected from hymexazole, octhilinone;
F.III-2) DNA topisomerase inhibitors selected from oxolinic acid;
F.III-3) Nucleotide metabolism inhibitors including hydroxy (2-amino)-pyrimidines selected from bupirimate;
F.IV) Inhibitors of cell division and or cytoskeleton
F.IV-1) Tubulin inhibitors:
   including benzimidazoles and thiophanates selected from benomyl, carbendazim, fuberidazole, thiabendazole, thiophanate-methyl;
   and including triazolopyrimidines selected from 5-chloro-7 (4-methylpiperidin-1-yl)-6-(2,4,6-trifluorophenyl)-[1,2,4]triazolo[1,5a]pyrimidine
F.IV-2) Other cell division inhibitors
   including benzamides and phenyl acetamides selected from diethofencarb, ethaboxam, pencycuron, fluopicolide, zoxamide;
F.IV-3) Actin inhibitors including benzophenones selected from metrafenone; pyriofenone;
F.V) Inhibitors of amino acid and protein synthesis
F.V-1) Methionine synthesis inhibitors including anilino-pyrimidines selected from cyprodinil, mepanipyrim, nitrapyrin, pyrimethanil;
F.V-2) Protein synthesis inhibitors including antibiotics selected from blasticidin-S, kasugamycin, kasugamycin hydrochloride-hydrate, mildiomycin, streptomycin, oxytetracyclin, polyoxine, validamycin A;
F.VI) Signal transduction inhibitors
F.VI-1) MAP/Histidine kinase inhibitors including dicarboximides selected from fluoroimid, iprodione, procymidone, vinclozolin;
   and including phenylpyrroles selected from fenpiclonil, fludioxonil;
F.VI-2) G protein inhibitors including quinolines selected from quinoxyfen;
F.VII) Lipid and membrane synthesis inhibitors
F.VII-1) Phospholipid biosynthesis inhibitors including organophosphorus compounds selected from edifenphos, iprobenfos, pyrazophos;
   and including dithiolanes selected from isoprothiolane;
F.VII-2) Lipid peroxidation
   including aromatic hydrocarbons selected from dicloran, quintozene, tecnazene, tolclofos-methyl, biphenyl, chloroneb, etridiazole;
F.VII-3) Carboxyl acid amides (CAA fungicides)
   including cinnamic or mandelic acid amides selected from dimethomorph, flumorph, mandiproamid, pyrimorph;
   and including valinamide carbamates selected from benthiavalicarb, iprovalicarb, pyribencarb, valifenalate and N-(1-(1-(4-cyano-phenyl)ethanesulfonyl)-but-2-yl) carbamic acid-(4-fluorophenyl) ester;
F.VII-4) Compounds affecting cell membrane permeability and fatty acides including carbamates selected from propamocarb, propamocarb-hydrochlorid,
F.VII-5) fatty acid amide hydrolase inhibitors: 1-[4-[4-[5-(2,6-difluorophenyl)-4,5-dihydro-3 isoxazolyl]-2-thiazolyl]-1-piperidinyl]-2-[5-methyl-3-(trifluoromethyl)-1H-pyrazol-1 yl]ethanone;
F.VIII) Inhibitors with Multi Site Action
F.VIII-1) Inorganic active substances selected from Bordeaux mixture, copper acetate, copper hydroxide, copper oxychloride, basic copper sulfate, sulfur;
F.VIII-2) Thio- and dithiocarbamates selected from ferbam, mancozeb, maneb, metam, methasulphocarb, metiram, propineb, thiram, zineb, ziram;
F.VIII-3) Organochlorine compounds including phthalimides, sulfamides, chloronitriles selected from anilazine, chlorothalonil, captafol, captan, folpet, dichlofluanid, dichlorophen, flusulfamide, hexachlorobenzene, pentachlorphenole and its salts, phthalide, tolylfluanid, N-(4-chloro-2-nitro-phenyl)-N-ethyl-4-methylbenzenesulfonamide;
F.VIII-4) Guanidines selected from guanidine, dodine, dodine free base, guazatine, guazatine-acetate, iminoctadine, iminoctadine-triacetate, iminoctadinetris(albesilate), dithianon, 2,6-dimethyl-1H,5H-[1,4]dithiino[2,3-c:5,6-c′]dipyrrole-1,3,5,7(2H,6H)-tetraone;
F.VIII-5) Ahtraquinones selected from dithianon;
F.IX) Cell wall synthesis inhibitors
F.IX-1) Inhibitors of glucan synthesis selected from validamycin, polyoxin B;
F.IX-2) Melanin synthesis inhibitors selected from pyroquilon, tricyclazole, carpropamide, dicyclomet, fenoxanil;
F.X) Plant defence inducers
F.X-1) Salicylic acid pathway selected from acibenzolar-S-methyl;
F.X-2) Others selected from probenazole, isotianil, tiadinil, prohexadione-calcium; including phosphonates selected from fosetyl, fosetyl-aluminum, phosphorous acid and its salts;
F.XI) Unknown mode of action:
   bronopol, chinomethionat, cyflufenamid, cymoxanil, dazomet, debacarb, diclomezine, difenzoquat, difenzoquat-methylsulfate, diphenylamin, fenpyrazamine, flumetover, flusulfamide, flutianil, methasulfocarb, nitrapyrin, nitrothalisopropyl, oxathiapiprolin, oxin-copper, proquinazid, tebufloquin, tecloftalam, triazoxide, 2-butoxy-6-iodo-3-propylchromen-4-one, N-(cyclopropylmethoxyimino-(6-difluoro-methoxy-2,3-difluoro-phenyl)-methyl)-2-phenyl acetamide, N′-(4-(4-chloro-3-trifluoromethyl-phenoxy)-2,5-dimethyl-phenyl)-N-ethyl-N methyl formamidine, N′ (4-(4-fluoro-3-trifluoromethyl-phenoxy)-2,5-dimethyl-phenyl)-N-ethyl-N-methyl formamidine, N′-(2-methyl-5-trifluoromethyl-4-(3-trimethylsilanyl-propoxy)-phenyl)-N-ethyl-N-methyl formamidine, N′-(5-difluoromethyl-2 methyl-4-(3-trimethylsilanyl-propoxy)phenyl)-N-ethyl-N-methyl formamidine, 2-{1-[2-(5-methyl-3-trifluoromethylpyrazole-1-yl)-acetyl]-piperidin-4-yl}-thiazole-4-carboxylic acid methyl-(1,2,3,4-tetrahydro-naphthalen-1-yl)-amide, 2-{1-[2-(5-methyl-3-trifluoromethylpyrazole-1-yl)-acetyl]-piperidin-4-yl}-thiazole-4-carboxylic acid methyl-(R)-1,2,3,4-tetrahydro-naphthalen-1-yl-amide, methoxy-acetic acid 6-tert-butyl-8-fluoro-2,3-dimethyl-quinolin-4-yl ester and N-Methyl-2-{1-[(5-methyl-3-trifluoromethyl-1H-pyrazol-1-yl)-acetyl]-piperidin-4-yl}-N-[(1R)-1,2,3,4-tetrahydronaphthalen-1-yl]-4-thiazolecarboxamide, 3-[5-(4-chloro-phenyl)-2,3-dimethyl-isoxazolidin-3 yl]-pyridine, pyrisoxazole, 5-amino-2-isopropyl-3-oxo-4-ortho-tolyl-2,3-dihydro-pyrazole-1 carbothioic acid S-allyl ester, N-(6-methoxy-pyridin-3-yl)cyclopropanecarboxylic acid amide, 5-chloro-1 (4,6-dimethoxy-pyrimidin-2-yl)-2-methyl-1H-benzoimidazole, 2-(4-chloro-phenyl)-N-[4-(3,4-dimethoxy-phenyl)-isoxazol-5-yl]-2-prop-2-ynyloxy-acetamide, ethyl (Z) 3 amino-2-cyano-3-phenyl-prop-2-enoate, tert-butyl N-[6-[[(Z)-[(1-methyltetrazol-5-yl)-phenyl-methylene]amino]oxymethyl]-2-pyridyl]carbamate, pentyl N-[6-[[(Z)-[(1-methyltetrazol-5-yl)-phenyl-methylene]amino]oxymethyl]-2-pyridyl]carbamate, 2-[2-[(7,8-difluoro-2-methyl-3-quinolyl)oxy]-6-fluoro-phenyl]propan-2-ol, 2-[2-fluoro-6-[(8-fluoro-2-methyl-3-quinolyl)oxy]phenyl]propan-2-ol, 3-(5-fluoro-3,3,4,4-tetramethyl-3,4-dihydroiso [symbol] quinolin-1-yl)quinoline, 3-(4,4-difluoro-3,3-dimethyl-3,4-dihydroisoquinolin-1-yl)-quinoline, 3-(4,4,5-trifluoro-3,3-dimethyl-3,4-dihydroisoquinolin-1-yl)quinoline;
F.XI) Growth regulators:
   abscisic acid, amidochlor, ancymidol, 6-benzylaminopurine, brassinolide, butralin, chlormequat (chlormequat chloride), choline chloride, cyclanilide, daminozide, dikegulac, dimethipin, 2,6-dimethylpuridine, ethephon, flumetralin, flurprimidol, fluthiacet, forchlorfenuron, gibberellic acid, inabenfide, indole-3-acetic acid, maleic hydrazide, mefluidide, mepiquat (mepiquat chloride), naphthaleneacetic acid, N 6 benzyladenine, paclobutrazol, prohexadione (prohexadione-calcium), prohydrojasmon, thidiazuron, triapenthenol, tributyl phosphorotrithioate, 2,3,5 tri iodobenzoic acid, trinexapac-ethyl and uniconazole;
F.XIII) Biopesticides
F.XIII-1) Microbial pesticides with fungicidal, bactericidal, viricidal and/or plant defense activator activity: Ampelomyces quisqualis, Aspergillus flavus, Aureobasidium pullulans, Bacillus amyloliquefaciens, B. mojavensis, B. pumilus, B. simplex, B. solisalsi, B. subtilis, B. subtilis var. amyloliquefaciens, Candida oleophila, C. saitoana, Clavibacter michiganensis (bacteriophages), Coniothyrium minitans, Cryphonectria parasitica, Cryptococcus albidus, Fusarium oxysporum, Clonostachys rosea f. catenulate (also named Gliocladium catenulatum), Gliocladium roseum, Metschnikowia fructicola, Microdochium dimerum, Paeni-bacillus polymyxa, Pantoea agglomerans, Phlebiopsis gigantea, Pseudozyma flocculosa, Pythium oligandrum, Sphaerodes mycoparasitica, Streptomyces lydicus, S. violaceusniger, Talaromyces flavus, Trichoderma asperellum, T. atroviride, T. fertile, T. gamsii, T. harmatum; mixture of T. harzianum and T. viride; mixture of T. polysporum and T. harzianum; T. stromaticum, T. virens (also named Gliocladium virens), T. viride, Typhula phacorrhiza, Ulocladium oudema, U. oudemansii, Verticillium dahlia, zucchini yellow mosaic virus (avirulent strain);
F.XIII-2) Biochemical pesticides with fungicidal, bactericidal, viricidal and/or plant defense activator activity: chitosan (hydrolysate), jasmonic acid or salts or derivatives thereof, laminarin, Menhaden fish oil, natamycin, Plum pox virus coat protein, Reynoutria sachlinensis extract, salicylic acid, tea tree oil;
F.XIII-3) Microbial pesticides with plant stress reducing, plant growth regulator, plant growth promoting and/or yield enhancing activity: Azospirillum amazonense A. brasilense, A. lipoferum, A. irakense, A. halopraeferens, Bradyrhizobium sp., B. japonicum, Glomus intraradices, Mesorhizobium sp., Paenibacillus alvei, Penicillium bilaiae, Rhizobium leguminosarum bv. phaseolii, R. I. trifolii, R. I. bv. viciae, Sinorhizobium meliloti;
F.XIII-4) Biochemical pesticides with plant stress reducing, plant growth regulator and/or plant yield enhancing activity: abscisic acid, aluminium silicate (kaolin), 3-decen-2-one, homobrassinlide, humates, lysophosphatidyl ethanolamine, polymeric polyhydroxy acid, Ascophyllum nodosum (Norwegian kelp, Brown kelp) extract and Ecklonia maxima (kelp) extract.

The commercially available compounds II of the group F listed above may be found in The Pesticide Manual, 15th Edition, C. D. S. Tomlin, British Crop Protection Council (2011) among other publications. Their preparation and their activity against harmful fungi is known (cf.: http://www.alanwood.net/pesticides/); these substances are commercially available. The compounds described by IUPAC nomenclature, their preparation and their fungicidal activity are also known (cf. Can. J. Plant Sci. 48(6), 587-94, 1968; EP A 141 317; EP-A 152 031; EP-A 226 917; EP A 243 970; EP A 256 503; EP-A 428 941; EP-A 532 022; EP-A 1 028 125; EP-A 1 035 122; EP A 1 201 648; EP A 1 122 244, JP 2002316902; DE 19650197; DE 10021412; DE 102005009458; U.S. Pat. No. 3,296,272; U.S. Pat. No. 3,325,503; WO 98/46608; WO 99/14187; WO 99/24413; WO 99/27783; WO 00/29404; WO 00/46148; WO 00/65913; WO 01/54501; WO 01/56358; WO 02/22583; WO 02/40431; WO 03/10149; WO 03/11853; WO 03/14103; WO 03/16286; WO 03/53145; WO 03/61388; WO 03/66609; WO 03/74491; WO 04/49804; WO 04/83193; WO 05/120234; WO 05/123689; WO 05/123690; WO 05/63721; WO 05/87772; WO 05/87773; WO 06/15866; WO 06/87325; WO 06/87343; WO 07/82098; WO 07/90624, WO 11/028657).

The biopesticides of group F.XIII are disclosed above in the paragraphs about biopesticides from group II-M.Y.

### Formulations

The invention also relates to agrochemical compositions suitable for applying in soil treatment methods comprising an auxiliary and at least one compound of formula (I) according to the invention.

An agrochemical composition comprises a pesticidally effective amount of a compound of formula (I). The term “effective amount” denotes an amount of the composition or of the compounds I alone or in combination with compound II, which is sufficient for controlling harmful pests on cultivated plants or in the protection of materials and which does not result in a substantial damage to the treated plants. Such an amount can vary in a broad range and is dependent on various factors, such as the animal pests species to be controlled, the treated cultivated plant or material, the climatic conditions and the specific compound I used.

The compound of formula (I), their N-oxides and salts can be converted into customary types of agrochemical compositions, e. g. solutions, emulsions, suspensions, dusts, powders, pastes, granules, pressings, capsules, and mixtures thereof. Examples for composition types are suspensions (e.g. SC, OD, FS), emulsifiable concentrates (e.g. EC), emulsions (e.g. EW, EO, ES, ME), capsules (e.g. CS, ZC), pastes, pastilles, wettable powders or dusts (e.g. WP, SP, WS, DP, DS), pressings (e.g. BR, TB, DT), granules (e.g. WG, SG, GR, FG, GG, MG), insecticidal articles (e.g. LN), as well as gel formulations for the treatment of plant propagation materials such as seeds (e.g. GF). These and further compositions types are defined in the “Catalogue of pesticide formulation types and international coding system”, Technical Monograph No. 2, 6th Ed. May 2008, CropLife International.

The compositions are prepared in a known manner, such as described by Mollet and Grubemann, Formulation technology, Wiley VCH, Weinheim, 2001; or Knowles, New developments in crop protection product formulation, Agrow Reports DS243, T&F Informa, London, 2005. Suitable auxiliaries are solvents, liquid carriers, solid carriers or fillers, surfactants, dispersants, emulsifiers, wetters, adjuvants, solubilizers, penetration enhancers, protective colloids, adhesion agents, thickeners, humectants, repellents, attractants, feeding stimulants, compatibilizers, bactericides, anti-freezing agents, anti-foaming agents, colorants, tackifiers and binders.

Suitable solvents and liquid carriers are water and organic solvents, such as mineral oil fractions of medium to high boiling point, e.g. kerosene, diesel oil; oils of vegetable or animal origin; aliphatic, cyclic and aromatic hydrocarbons, e. g. toluene, paraffin, tetrahydronaphthalene, alkylated naphthalenes; alcohols, e.g. ethanol, propanol, butanol, benzylalcohol, cyclo-hexanol; glycols; DMSO; ketones, e.g. cyclohexanone; esters, e.g. lactates, carbonates, fatty acid esters, gamma-butyrolactone; fatty acids; phosphonates; amines; amides, e.g. N-methylpyrrolidone, fatty acid dimethylamides; and mixtures thereof.

Suitable solid carriers or fillers are mineral earths, e.g. silicates, silica gels, talc, kaolins, limestone, lime, chalk, clays, dolomite, diatomaceous earth, bentonite, calcium sulfate, magnesium sulfate, magnesium oxide; polysaccharides, e.g. cellulose, starch; fertilizers, e.g. ammonium sulfate, ammonium phosphate, ammonium nitrate, ureas; products of vegetable origin, e.g. cereal meal, tree bark meal, wood meal, nutshell meal, and mixtures thereof.

Suitable surfactants are surface-active compounds, such as anionic, cationic, nonionic and amphoteric surfactants, block polymers, polyelectrolytes, and mixtures thereof. Such surfactants can be used as emulsifier, dispersant, solubilizer, wetter, penetration enhancer, protective colloid, or adjuvant. Examples of surfactants are listed in McCutcheon's, Vol. 1: Emulsifiers & Detergents, McCutcheon's Directories, Glen Rock, USA, 2008 (International Ed. or North American Ed.).

Suitable anionic surfactants are alkali, alkaline earth or ammonium salts of sulfonates, sulfates, phosphates, carboxylates, and mixtures thereof. Examples of sulfonates are alkylarylsulfonates, diphenylsulfonates, alpha-olefin sulfonates, lignine sulfonates, sulfonates of fatty acids and oils, sulfonates of ethoxylated alkylphenols, sulfonates of alkoxylated arylphenols, sulfonates of condensed naphthalenes, sulfonates of dodecyl- and tridecylbenzenes, sulfonates of naphthalenes and alkyl-naphthalenes, sulfosuccinates or sulfosuccinamates. Examples of sulfates are sulfates of fatty acids and oils, of ethoxylated alkylphenols, of alcohols, of ethoxylated alcohols, or of fatty acid esters. Examples of phosphates are phosphate esters. Examples of carboxylates are alkyl carboxylates, and carboxylated alcohol or alkylphenol ethoxylates.

Suitable nonionic surfactants are alkoxylates, N-substituted fatty acid amides, amine oxides, esters, sugar-based surfactants, polymeric surfactants, and mixtures thereof. Examples of alkoxylates are compounds such as alcohols, alkylphenols, amines, amides, arylphenols, fatty acids or fatty acid esters which have been alkoxylated with 1 to 50 equivalents. Ethylene oxide and/or propylene oxide may be employed for the alkoxylation, preferably ethylene oxide. Examples of N-substituted fatty acid amides are fatty acid glucamides or fatty acid alkanolamides. Examples of esters are fatty acid esters, glycerol esters or monoglycerides. Examples of sugar-based surfactants are sorbitans, ethoxylated sorbitans, sucrose and glucose esters or alkylpolyglucosides. Examples of polymeric surfactants are home- or copolymers of vinylpyrrolidone, vinylalcohols, or vinylacetate.

Suitable cationic surfactants are quaternary surfactants, for example quaternary ammonium compounds with one or two hydrophobic groups, or salts of long-chain primary amines. Suitable amphoteric surfactants are alkylbetains and imidazolines. Suitable block polymers are block polymers of the A-B or A-B-A type comprising blocks of polyethylene oxide and polypropylene oxide, or of the A-B-C type comprising alkanol, polyethylene oxide and polypropylene oxide. Suitable polyelectrolytes are polyacids or polybases. Examples of polyacids are alkali salts of polyacrylic acid or polyacid comb polymers. Examples of polybases are polyvinylamines or polyethyleneamines.

Suitable adjuvants are compounds, which have a neglectable or even no pesticidal activity themselves, and which improve the biological performance of the compound I on the target. Examples are surfactants, mineral or vegetable oils, and other auxiliaries. Further examples are listed by Knowles, Adjuvants and additives, Agrow Reports DS256, T&F Informa UK, 2006, chapter 5.

Suitable thickeners are polysaccharides (e.g. xanthan gum, carboxymethylcellulose), anorganic clays (organically modified or unmodified), polycarboxylates, and silicates.

Suitable bactericides are bronopol and isothiazolinone derivatives such as alkylisothiazolinones and benzisothiazolinones.

Suitable anti-freezing agents are ethylene glycol, propylene glycol, urea and glycerin.

Suitable anti-foaming agents are silicones, long chain alcohols, and salts of fatty acids.

Suitable colorants (e.g. in red, blue, or green) are pigments of low water solubility and water-soluble dyes. Examples are inorganic colorants (e.g. iron oxide, titan oxide, iron hexacyanoferrate) and organic colorants (e.g. alizarin-, azo- and phthalocyanine colorants).

Suitable tackifiers or binders are polyvinylpyrrolidons, polyvinylacetates, polyvinyl alcohols, polyacrylates, biological or synthetic waxes, and cellulose ethers.

The agrochemical compositions generally comprise between 0.01 and 95%, preferably between 0.1 and 90%, and in particular between 0.5 and 75%, by weight of active substance. The active substances are employed in a purity of from 90% to 100%, preferably from 95% to 100% (according to NMR spectrum).

The compounds and mixtures according to the invention are especially suitable for use in seed treatment. Solutions for seed treatment (LS), Suspoemulsions (SE), flowable concentrates (FS), powders for dry treatment (DS), water-dispersible powders for slurry treatment (WS), water-soluble pow-ders (SS), emulsions (ES), emulsifiable concentrates (EC) and gels (GF) are usually employed for the purposes of treatment of plant propagation materials, particularly seeds. The composi-tions in question give, after two-to-tenfold dilution, active substance concentrations of from 0.01 to 60% by weight, preferably from 0.1 to 40% by weight, in the ready-to-use preparations. Appli-cation can be carried out before or during sowing. Methods for applying compound I and com-positions thereof, respectively, on to plant propagation material, especially seeds include dress-ing, coating, pelleting, dusting, soaking and in-furrow application methods of the propagation material. Preferably, compound I or the compositions thereof, respectively, are applied on to the plant propagation material by a method such that germination is not induced, e. g. by seed dressing, pelleting, coating and dusting.

When employed in plant protection, the amounts of active substances applied are, depending on the kind of effect desired, from 0.001 to 2 kg per ha, preferably from 0.005 to 2 kg per ha, more preferably from 0.05 to 0.9 kg per ha, and in particular from 0.1 to 0.75 kg per ha.

In treatment of plant propagation materials such as seeds, e. g. by dusting, coating or drenching seed, amounts of active substance of from 0.1 to 1000 g, preferably from 1 to 1000 g, more preferably from 1 to 100 g and most preferably from 5 to 100 g, per 100 kilogram of plant propagation material (preferably seeds) are generally required.

When used in the protection of materials or stored products, the amount of active substance applied depends on the kind of application area and on the desired effect. Amounts customarily applied in the protection of materials are 0.001 g to 2 kg, preferably 0.005 g to 1 kg, of active substance per cubic meter of treated material.

Various types of oils, wetters, adjuvants, fertilizer, or micronutrients, and further pesticides (e.g. herbicides, insecticides, fungicides, growth regulators, safeners) may be added to the active substances or the compositions com [symbol] prising them as premix or, if appropriate not until immediately prior to use (tank mix). These agents can be admixed with the compositions according to the invention in a weight ratio of 1:100 to 100:1, preferably 1:10 to 10:1.

Usually, the agrochemical composition is made up with water, buffer, and/or further auxiliaries to the desired applica-tion concentration and the ready-to-use spray liquor or the agrochemical composition according to the invention is thus obtained.

According to one embodiment, individual components of the composition according to the invention such as parts of a kit or parts of a binary or ternary mixture may be mixed by the user himself in a spray tank and further auxiliaries may be added, if appropriate.

In a further embodiment, either individual components of the composition according to the invention or partially premixed components, e. g. components comprising compounds I and/or active compound II, may be mixed by the user in a spray tank and further auxiliaries and additives may be added, if appropriate.

In a further embodiment, either individual components of the composition according to the invention or partially premixed components, e. g. components comprising compounds I and/or active compound II, can be applied jointly (e.g. after tank mix) or consecutively.

### Applications

### Soil Treatment

The present invention relates to the methods by use on natural substrates (soil) or artificial (growth) substrates (e.g. rock wool, glass wool, quartz sand, gravel, expanded clay, vermiculite), in the open or in closed systems (e.g. greenhouses or under film mulch) and in annual crops (such as vegetables, spices, ornamentals) or perennial crops (such as citrus plants, fruits, tropical crops, spices, nuts, grapevines, conifers and ornamentals).

It has now been found that the problems associated with combating soil-living pests by pesticide treatment of the soil can be overcome by such application methods using compounds of the present invention.

The animal pest, i.e. the insects, arachnids and nematodes, the plant, the water or the soil in which the plant is growing can be contacted with the present compounds of formula I or composition(s) containing them by any application method known in the art. As such, “contacting” includes both direct contact (applying the compounds/compositions directly on the animal pest or plant) and indirect contact (applying the compounds/compositions to the locus of the animal pest or plant). When the plant is contacted, typically the tuber, bulbs or roots of the plant are contacted.

Soil application techniques and soil application methods according to the present invention, are methods wherein the active compound(s) are applied by drenching the soil, applied by drip irrigation, applied by soil injection.

Another soil application technique in the sense of the present invention is a method, wherein the active compound(s) are applied by dipping roots, tubers or bulbs.

An alternative method of soil application technique is that the active compound(s) are applied with drip application systems.

In the case of soil treatment or of application to the pests dwelling place or nest, the quantity of active ingredient ranges from 0.0001 to 500 g per 100 m², preferably from 0.001 to 20 g per 100 m².

### Seed Treatment

The compounds of formula I are especially also suitable for the treatment of seeds in order to protect the seed from insect pest, in particular from soil-living insect pests and the resulting plant's roots and shoots against soil pests and foliar insects.

The compounds of formula I are particularly useful for the protection of the seed from soil pests and the resulting plant's roots and shoots against soil pests and foliar insects. The protection of the resulting plant's roots and shoots is preferred. More preferred is the protection of resulting plant's shoots from piercing and sucking insects, wherein the protection from aphids is most preferred.

The present invention therefore comprises a method for the protection of seeds from insects, in particular from soil insects and of the seedling's roots and shoots from insects, in particular from soil and foliar insects, said method comprising contacting the seeds before sowing and/or after pregermination with a compound of the general formula I or a salt thereof. Particularly preferred is a method, wherein the plant's roots and shoots are protected, more preferably a method, wherein the plants shoots are protected form piercing and sucking insects, most preferably a method, wherein the plants shoots are protected from aphids.

Consequently, the present invention relates to methods for the protection of seeds, from soil insects and of the resulting plant's roots and shoots from soil and foliar insects wherein the seeds are contacted before sowing and/or after pregermination with the neonicotinoid insecticide cycloxaprid alone or in combination with a selected pesticidal active compound II.

The term seed embraces seeds and plant propagules of all kinds including but not limited to true seeds, seed pieces, suckers, corms, bulbs, fruit, tubers, grains, cuttings, cut shoots and the like and means in a preferred embodiment true seeds.

The term seed treatment comprises all suitable seed treatment techniques known in the art, such as seed dressing, seed coating, seed dusting, seed soaking and seed pelleting. The present invention also comprises seeds coated with or containing the active compound.

The term “coated with and/or containing” generally signifies that the active ingredient is for the most part on the surface of the propagation product at the time of application, although a greater or lesser part of the ingredient may penetrate into the propagation product, depending on the method of application. When the said propagation product is (re)planted, it may absorb the active ingredient.

In general, suitable seeds are seeds of cereals, root crops, oil crops, vegetables, spices, ornamentals, for example seed of durum and other wheat, barley, oats, rye, maize (fodder maize and sugar maize/sweet and field corn), soybeans, oil crops, crucifers, cotton, sunflowers, bananas, rice, oilseed rape, turnip rape, sugarbeet, fodder beet, eggplants, potatoes, grass, lawn, turf, fodder grass, tomatoes, leeks, pumpkin/squash, cabbage, iceberg lettuce, pepper, cucumbers, melons, Brassica species, melons, beans, peas, garlic, onions, carrots, tuberous plants such as potatoes, sugar cane, tobacco, grapes, petunias, geranium/pelargoniums, pansies and impatiens.

The seed treatment application of the active compound is carried out by spraying or by dusting the seeds before sowing of the plants and before emergence of the plants.

Compositions which are especially useful for seed treatment are e.g.:

A Soluble concentrates (SL, LS)

### D Emulsions (EW, EO, ES)

### E Suspensions (SC, OD, FS)

F Water-dispersible granules and water-soluble granules (WG, SG)
G Water-dispersible powders and water-soluble powders (WP, SP, WS)

### H Gel-Formulations (GF)

I Dustable powders (DP, DS)

Conventional seed treatment formulations include for example flowable concentrates FS, solutions LS, powders for dry treatment DS, water dispersible powders for slurry treatment WS, water-soluble powders SS and emulsion ES and EC and gel formulation GF. These formulations can be applied to the seed diluted or undiluted. Application to the seeds is carried out before sowing, either directly on the seeds or after having pregerminated the latter

In a preferred embodiment a FS formulation is used for seed treatment. Typically, a FS formulation may comprise 1-800 g/l of active ingredient, 1-200 g/l Surfactant, 0 to 200 g/l antifreezing agent, 0 to 400 g/l of binder, 0 to 200 g/l of a pigment and up to 1 liter of a solvent, preferably water.

Especially preferred FS formulations of compounds of formula I for seed treatment usually comprise from 0.1 to 80% by weight (1 to 800 g/l) of the active ingredient, from 0.1 to 20% by weight (1 to 200 g/l) of at least one surfactant, e.g. 0.05 to 5% by weight of a wetter and from 0.5 to 15% by weight of a dispersing agent, up to 20% by weight, e.g. from 5 to 20% of an anti-freeze agent, from 0 to 15% by weight, e.g. 1 to 15% by weight of a pigment and/or a dye, from 0 to 40% by weight, e.g. 1 to 40% by weight of a binder (sticker/adhesion agent), optionally up to 5% by weight, e.g. from 0.1 to 5% by weight of a thickener, optionally from 0.1 to 2% of an anti-foam agent, and optionally a preservative such as a biocide, antioxidant or the like, e.g. in an amount from 0.01 to 1% by weight and a filler/vehicle up to 100% by weight.

Seed Treatment formulations may additionally also comprise binders and optionally colorants.

Binders can be added to improve the adhesion of the active materials on the seeds after treatment. Suitable binders are homo- and copolymers from alkylene oxides like ethylene oxide or propylene oxide, polyvinylacetate, polyvinylalcohols, polyvinylpyrrolidones, and copolymers thereof, ethylene-vinyl acetate copolymers, acrylic homo- and copolymers, polyethyleneamines, polyethyleneamides and polyethyleneimines, polysaccharides like celluloses, tylose and starch, polyolefin homo- and copolymers like olefin/maleic anhydride copolymers, polyurethanes, polyesters, polystyrene homo and copolymers

Optionally, also colorants can be included in the formulation. Suitable colorants or dyes for seed treatment formulations are Rhodamin B, C.I. Pigment Red 112, C.I. Solvent Red 1, pigment blue 15:4, pigment blue 15:3, pigment blue 15:2, pigment blue 15:1, pigment blue 80, pigment yellow 1, pigment yellow 13, pigment red 112, pigment red 48:2, pigment red 48:1, pigment red 57:1, pigment red 53:1, pigment orange 43, pigment orange 34, pigment orange 5, pigment green 36, pigment green 7, pigment white 6, pigment brown 25, basic violet 10, basic violet 49, acid red 51, acid red 52, acid red 14, acid blue 9, acid yellow 23, basic red 10, basic red 108.

Examples of a gelling agent is carrageen (Satiagel®)

In the treatment of seed, the application rates of the compounds I are generally from 0.1 g to 10 kg per 100 kg of seed, preferably from 1 g to 5 kg per 100 kg of seed, more preferably from 1 g to 1000 g per 100 kg of seed and in particular from 1 g to 200 g per 100 kg of seed.

The invention therefore also relates to seed comprising a compound of the formula I, or an agriculturally useful salt of I, as defined herein. The amount of the compound I or the agriculturally useful salt thereof will in general vary from 0.1 g to 10 kg per 100 kg of seed, preferably from 1 g to 5 kg per 100 kg of seed, in particular from 1 g to 1000 g per 100 kg of seed. For specific crops such as lettuce the rate can be higher.

In general, “pesticidally effective amount” means the amount of active ingredient needed to achieve an observable effect on growth, including the effects of necrosis, death, retardation, prevention, and removal, destruction, or otherwise diminishing the occurrence and activity of the target organism. The pesticidally effective amount can vary for the various compounds/compositions used in the invention. A pesticidally effective amount of the compositions will also vary according to the prevailing conditions such as desired pesticidal effect and duration, weather, target species, locus, mode of application, and the like.

The compounds of formula I or the pesticidal compositions comprising them may be used to protect growing plants and crops from attack or infestation by animal pests, especially insects, acaridae or arachnids by contacting the plant/crop with a pesticidally effective amount of compounds of formula I. The term “crop” refers both to growing and harvested crops.

Thus, as with regards to the use and for the purpose of the present invention, vegetables are to be understood as meaning for example fruiting vegetables and inflorescences as vegetables, i.e. bell peppers, chillies, tomatoes, aubergines, cucumbers, pumpkins, courgettes, broad beans, climbing and dwarf beans, peas, artichokes and maize. Further also leafy vegetables like head-forming lettuce, chicory, endives, various types of cress, of rocket, lamb's lettuce, iceberg lettuce, leeks, spinach and chard. Furthermore tuber vegetables, root vegetables and stem vegetables, like celeriac/celery, beetroot, carrots, radish, horseradish, scorzonera, asparagus, beet for human consumption, palm hearts and bamboo shoots. Further also bulb vegetables like onions, leeks, fennel and garlic. Brassica vegetables such as cauliflower, broccoli, kohlrabi, red cabbage, white cabbage, curly kale, Savoy cabbage, Brussels sprouts and Chinese cabbage are also vegetable in the sense of the present application.

Regarding the use and for the purpose of the present invention, perennial crops are to be understood as meaning citrus, for example, oranges, grapefruits, tangerines, lemons, limes, Seville oranges, cumquats and satsumas. Also pome fruit such as, for example, apples, pears and quinces, and stone fruit such as, for example, peaches, nectarines, cherries, plums, quetsch, apricots. Further grapevines, hops, olives, tea and tropical crops such as, for example, mangoes, papayas, figs, pineapples, dates, bananas, durians, kaki fruit, coconuts, cacao, coffee, avocados lychees, maracujas, and. guavas. Furthermore soft fruit such as, for example, currants, gooseberries, raspberries, blackberries, blueberries, strawberries, cranberries, kiwi fruit and American cranberries. Almonds and nuts such as, for example, hazelnuts, walnuts, pistachios, cashew nuts, para nuts, pecan nuts, butternuts, chestnuts, hickory nuts, macadamia nuts and peanuts are also fruits in the sense of the present invention.

As with regard to the use and for the purpose of the present invention, ornamentals are understood as meaning annual and perennial plants, for example cut flowers such as, for example, roses, carnations, gerbera, lilies, marguerites, chrysanthemums, tulips, narcissus, anemones, poppies, amaryllis, dahlias, azaleas, hibiscus, but also for example border plants, pot plants and perennials such as, for example, roses, Tagetes, violas, geraniums, fuchsias, hibiscus, chrysanthemum, busy lizzie, cyclamen, African violet, sunflowers, begonias.

Furthermore for example also bushes and conifers such as, for example, ficus, rhododendron, firs, spruces, pines, yews, juniper, umbrella pines, oleander.

As regards the use, spices are understood as meaning annual and perennial plants such as, for example, aniseed, chilli pepper, paprika, pepper, vanilla, marjoram, thyme, cloves, juniper berries, cinnamon, tarragon, coriander, saffron, ginger.

Furthermore the compounds of the present invention and the compositions comprising them are particularly important in the control of a multitude of insects on various cultivated plants, such as cereal and oil crops, for example seed of durum and other wheat, barley, oats, rye, maize (fodder maize and sugar maize/sweet and field corn), soybeans, oil crops, crucifers, cotton, bananas, rice, oilseed rape, turnip rape, sugarbeet, fodder beet, eggplants, potatoes, grass, lawn, turf, fodder grass, sugar cane or tobacco.

The compounds of the invention can also be applied preventively to places at which occurrence of the pests is expected.

“Locus” means a habitat, breeding ground, plant, seed, soil, area, material or environment in which a pest or parasite is growing or may grow.

Plants which can be treated with compound(s) of formula I/inventive mixture(s) in-clude all genetically modified plants or transgenic plants, e.g. crops which tolerate the action of herbicides or fungicides or insecticides owing to breeding, including genetic engineering methods, or plants which have modified characteristics in comparison with existing plants, which can be generated for example by traditional breeding methods and/or the generation of mutants, or by recombinant procedures.

The term “plant propagation material” is to be understood to denote all the generative parts of the plant such as seeds and vegetative plant material such as cuttings and tubers (e. g. potatoes), which can be used for the multiplication of the plant. This in-cludes seeds, roots, fruits, tubers, bulbs, rhizomes, shoots, sprouts and other parts of plants. Seedlings and young plants, which are to be transplanted after germination or after emergence from soil, may also be included. These young plants or these plant propagation materials may be treated and protected, optionally also prophylactically, with a plant protection compound either at or before planting or transplanting, by a total or partial treatment and by immersion or pouring

The term “cultivated plants” refers to “modified plants” and “transgenic plants”. “Modi-fied plants” are those which have been modified by conventional breeding techniques. “Transgenic plants are those, which genetic material has been so modified by the use of recombinant DNA techniques that under natural circumstances cannot readily be obtained by cross breeding, mutations or natural recombination. Typically, one or more genes have been integrated into the genetic material of a genetically modified plant in order to improve certain properties of the plant. Such genetic modifications also include but are not limited to targeted post-transtional modification of protein(s), oligo- or poly-peptides e. g. by glycosylation or polymer additions such as prenylated, acetylated or farnesylated moieties or PEG moieties.

Preferred plants, from which “modified plants” and/or “transgenic plants” can be derived can be selected from the group consisting of cereals such as wheat, barley, rye and oat, alfalfa, apples, banana, beet, broccoli, broccoli, Brussels sprouts, cabbage, canola (rapeseed), carrot, cauliflower, cherries, chickpea, Chinese cabbage, Chinese mus-tard, collard, cotton, cranberries, creeping bentgrass, cucumber, eggplant, flax, grape, grapefruit, kale, kiwi, kohlrabi, maize (corn), melon, mizuna, mustard, papaya, pea-nut, pears, pepper, persimmons, pigeonpea, pineapple, plum, plum, potato, raspber-ry, rice, rutabaga, sorghum, soybean, squash, straw-berries, sugar beet, sugarcane, sunflower, sweet corn, tobacco, tomato, turnip, walnut, watermelon and winter squash,

more preferably from the group consisting of alfalfa, barley, canola (rapeseed), cotton, maize (corn), papaya, potato, rice, sorghum, soybean, squash, sugar beet, tomato and cereals such as wheat, barley, rye and oat, most preferably, the plant is selected from soybean, tomatoes and cereals such as wheat, barley, rye and oat, utmost preferably from soybean and cereals such as wheat, barley, rye and oat.

The cultivated plants are plants, which comprise at least one trait. The term “trait” refers to a property, which is present in the plant either by genetic engineering or by conventional breeding techniques. Examples of traits are

herbicide tolerance,
insecticide resistance by expression of bacertial toxins,
fungicidal resistance or viral resistance or bacterial resistance,
antibiotic resistance,
stress tolerance,
maturation alteration,
content modification of chemicals present in the cultivated plant compared to the corre-sponding wild-type plant,
modified nutrient uptake,
and male sterility.

Principally, cultivated plants may also comprise combinations of the aforementioned traits, e.g. they may be tolerant to the action of herbicides and express bacertial toxins.

Principally, all cultivated plants may also provide combinations of the aforementioned properties, e.g. they may be tolerant to the action of herbicides and express bacertial toxins.

In the detailed description below, the term “plant” refers to a cultivated plant.

Tolerance to herbicides can be obtained by creating insensitivity at the site of action of the herbicide by expression of a target enzyme which is resistant to herbicide; rapid metabolism (conjugation or degradation) of the herbicide by expression of enzymes which inactivate herbicide; or poor uptake and translocation of the herbicide. Examples are the expression of enzymes which are tolerant to the herbicide in comparison to wild type enzymes, such as the expression of 5-enolpyruvylshikimate-3-phosphate syn-thase (EPSPS), which is tolerant to glyphosate (see e.g. Heck et. al, Crop Sci. 45, 2005, 329-339; Funke et. al, PNAS 103, 2006, 13010-13015; U.S. Pat. No. 5,188,642, U.S. Pat. No. 4,940,835, U.S. Pat. No. 5,633,435, U.S. Pat. No. 5,804,425, U.S. Pat. No. 5,627,061), the expression of glutamine synthase which is tolerant to glufosinate and bialaphos (see e.g. U.S. Pat. No. 5,646,024, U.S. Pat. No. 5,561,236) and DNA constructs coding for dicamba-degrading enzymes (see e.g. U.S. Pat. No. 7,105,724). Gene constructs can be obtained, for example, from micro-organism or plants, which are tolerant to said herbicides, such as the Agrobacterium strain CP4 EPSPS which is resistant to glyphosate; Streptomyces bacteria which are resistance to glufosinate; Arabidopsis, Daucus carotte, Pseudomonoas sp. or Zea mais with chimeric gene sequences coging for HDDP (see e.g. WO1996/38567, WO 2004/55191); Arabidopsis thaliana which is resistant to protox inhibitors (see e.g. US2002/0073443).

Preferably, the herbicide tolerant plant can be selected from cereals such as wheat, barley, rye, oat; canola, sorghum, soybean, rice, oil seed rape, sugar beet, sugarcane, grapes, lentils, sunflowers, alfalfa, pome fruits; stone fruits; peanuts; coffee; tea; straw-berries; turf; vegetables, such as tomatoes, potatoes, cucurbits and lettuce, more pref-erably, the plant is selected from soybean, tomatoes and cereals such as wheat, bar-ley, rye and oat, most preferably from soybean and cereals such as wheat, barley, rye and oat.

Examples of commercial available transgenic plants with tolerance to herbicides, are the corn varieties “Roundup Ready Corn”, “Roundup Ready 2” (Monsanto), “Agrisure GT”, “Agrisure GT/CB/LL”, “Agrisure GT/RW”, “Agrisure 3000GT” (Syngenta), “Yield-Gard VT Rootworm/RR2” and “YieldGard VT Triple” (Monsanto) with tolerance to glyphosate; the corn varieties “Liberty Link” (Bayer), “Herculex I”, “Herculex RW”, “Her-culex Xtra” (Dow, Pioneer), “Agrisure GT/CB/LL” and “Agrisure CB/LL/RW” (Syngenta) with tolerance to glufosinate; the soybean varieties “Roundup Ready Soybean” (Mon-santo) and “Optimum GAT” (DuPont, Pioneer) with tolerance to glyphosate; the cotton varieties “Roundup Ready Cotton” and “Roundup Ready Flex” (Monsanto) with toler-ance to glyphosate; the cotton variety “FiberMax Liberty Link” (Bayer) with tolerance to glufosinate; the cotton variety “BXN” (Calgene) with tolerance to bromoxynil; the canola varieties “Navigator” und “Compass” (Rhone-Poulenc) with bromoxynil tolerance; the canola varierty “Roundup Ready Canola” (Monsanto) with glyphosate tolerance; the canola variety “InVigor” (Bayer) with glufosinate tolerance; the rice variety “Liberty Link Rice” (Bayer) with glulfosinate tolerance and the alfalfa variety “Roundup Ready Alfalfa” with glyphosate tolerance. Further transgenic plants with herbicide are commonly known, for instance alfalfa, apple, eucalyptus, flax, grape, lentils, oil seed rape, peas, potato, rice, sugar beet, sunflower, tobacco, tomatom turf grass and wheat with toler-ance to glyphosate (see e.g. U.S. Pat. No. 5,188,642, U.S. Pat. No. 4,940,835, U.S. Pat. No. 5,633,435, U.S. Pat. No. 5,804,425, U.S. Pat. No. 5,627,061); beans, soybean, cotton, peas, potato, sunflower, tomato, tobacco, corn, sorghum and sugarcane with tolerance to dicamba (see e.g. U.S. Pat. No. 7,105,724 and U.S. Pat. No. 5,670,454); pepper, apple, tomato, hirse, sunflower, tobacco, potato, corn, cucumber, wheat and sorghum with tolerance to 2,4-D (see e.g. U.S. Pat. No. 6,153,401, U.S. Pat. No. 6,100,446, WO2005107437, U.S. Pat. No. 5,608,147 and U.S. Pat. No. 5,670,454); sugarbeet, potato, tomato and tobacco with tolerance to gluphosinate (see e.g. U.S. Pat. No. 5,646,024, U.S. Pat. No. 5,561,236); canola, barley, cot-ton, lettuce, melon, millet, oats, potato, rice, rye, sorghum, soybean, sugarbeet, sun-flower, tobacco, tomato and wheat with tolerance to acetolactate synthase (ALS) inhib-iting herbicides, such as triazolopyrimidine sulfonamides, sulfonylureas and imidazoli-nones (see e.g. U.S. Pat. No. 5,013,659, WO2006060634, U.S. Pat. No. 4,761,373, U.S. Pat. No. 5,304,732, U.S. Pat. No. 6,211,438, U.S. Pat. No. 6,211,439 and U.S. Pat. No. 6,222,100); cereal, sugar cane, rice, corn, tobacco, soybean, cotton, rapeseed, sugar beet and potato with tolerance to HPPD inhibitor herbicides (see e.g. WO2004/055191, WO199638567, WO1997049816 and U.S. Pat. No. 6,791,014); wheat, soybean, cotton, sugar beet, rape, rice, sorghum and sugar cane with tolerance to protoporphy-rinogen oxidase (PPO) inhibitor herbicides (see e.g. US2002/0073443, US20080052798, Pest Management Science, 61, 2005, 277-285). The methods of producing such transgenic plants are generally known to the person skilled in the art and are described, for example, in the publications mentioned above.

Plants, which are capable of synthesising one or more selectively acting bacterial tox-ins, comprise for example at least one toxin from toxin-producing bacteria, especially those of the genus Bacillus, in particular plants capable of synthesising one or more insecticidal proteins from Bacillus cereus or Bacillus popliae; or insecticidal proteins from Bacillus thuringiensis, such as delta.-endotoxins, e.g. CryIA(b), CryIA(c), CryIF, CryIF(a2), CryIIA(b), CryIIIA, CryIIIB(b1) or Cry9c, or vegetative insecticidal proteins (VIP), e.g. VIP1, VIP2, VIP3 or VIP3A; or insecticidal proteins of bacteria colonising nematodes, for example Photorhabdus spp. or Xenorhabdus spp., such as Photorhab-dus luminescens, Xenorhabdus nematophilus; toxins produced by animals, such as scorpion toxins, arachnid toxins, wasp toxins and other insect-specific neurotoxins; toxins produced by fungi, such as Streptomycetes toxins, plant lectins, such as pea lectins, barley lectins or snowdrop lectins; agglutinins; proteinase inhibitors, such as trypsine inhibitors, serine protease inhibitors, patatin, cystatin, papain inhibitors; ribo-some-inactivating proteins (RIP), such as ricin, maize-RIP, abrin, luffin, saporin or bry-odin; steroid metabolism enzymes, such as 3-hydroxysteroidoxidase, ecdysteroid-UDP-glycosyl-transferase, cholesterol oxidases, ecdysone inhibitors, HMG-COA-reductase, ion channel blockers, such as blockers of sodium or calcium channels, ju-venile hormone esterase, diuretic hormone receptors, stilbene synthase, bibenzyl syn-thase, chitinases and glucanases.

In the context of the present invention there are to be understood by .delta.-endotoxins, for example CryIA(b), CryIA(c), CryIF, CryIF(a2), CryIIA(b), CryIIIA, CryIIIB(b1) or Cry9c, or vegetative insecticidal proteins (VIP), for example VIP1, VIP2, VIP3 or VIP3A, expressly also hybrid toxins, truncated toxins and modified toxins. Hybrid toxins are produced recombinantly by a new combination of different domains of those proteins (see, for example, WO 02/15701). An example for a truncated toxin is a truncated CryIA(b), which is expressed in the Bt11 maize from Syngenta Seed SAS, as described below. In the case of modified toxins, one or more amino acids of the naturally occurring toxin are replaced. In such amino acid replacements, preferably non-naturally present protease recognition sequences are inserted into the toxin, such as, for example, in the case of CryIIIA055, a cathepsin-D-recognition sequence is inserted into a CryIIIA toxin (see WO 03/018810).

Examples of such toxins or transgenic plants capable of synthesising such toxins are disclosed, for example, in EP-A-0 374 753, WO 93/07278, WO 95/34656, EP-A-0 427 529, EP-A-451 878 and WO 03/052073.

The processes for the preparation of such transgenic plants are generally known to the person skilled in the art and are described, for example, in the publications mentioned above. CryI-type deoxyribonucleic acids and their preparation are known, for example, from WO 95/34656, EP-A0 367 474, EP-A-0 401 979 and WO 90/13651.

The toxin contained in the transgenic plants imparts to the plants tolerance to harmful insects. Such insects can occur in any taxonomic group of insects, but are especially commonly found in the beetles (Coleoptera), two-winged insects (Diptera) and butter-flies (Lepidoptera).

Preferably, the plant capable of expression of bacterial toxins is selected from cereals such as wheat, barley, rye, oat; canola, sorghum, soybean, rice, oil seed rape, sugar beet, sugarcane, grapes, lentils, sunflowers, alfalfa, pome fruits; stone fruits; peanuts; coffee; tea; strawberries; turf; vegetables, such as tomatoes, potatoes, cucurbits and lettuce, more preferably, the plant is selected from soybean, tomatoes and cereals such as wheat, barley, rye and oat, most preferably from soybean, maize and cereals such as wheat, barley, rye and oat.

Examples of commercial available transgenic plants capable of expression of bacterial toxins are the corn varieties “YieldGard corn rootworm” (Monsanto), “YieldGard VT” (Monsanto), “Herculex RW” (Dow, Pioneer), “Herculex Rootworm” (Dow, Pioneer) and “Agrisure CRW” (Syngenta) with resistance against corn rootworm; the corn varieties “YieldGard corn borer” (Monsanto), “YieldGard VT Pro” (Monsanto), “Agrisure CB/LL” (Syngenta), “Agrisure 3000GT” (Syngenta), “Hercules I”, “Hercules II” (Dow, Pioneer), “KnockOut” (Novartis), “NatureGard” (Mycogen) and “StarLink” (Aventis) with re-sistance against corn borer, the corn varieties “Herculex I” (Dow, Pioneer) and “Hercu-lex Xtra” (Dow, Pioneer) with resistance against western bean cutworm, corn borer, black cutworm and fall armyworm; the corn variety “YieldGard Plus” (Monsanto) with resistance against corn borer and corn rootworm; the cotton variety “Bollgard I”” (Mon-santo) with resistance against tobacco budworm; the cotton varieties “Bollgard II” (Monsanto), “WideStrike” (Dow) and “VipCot” (Syngenta) with resistance against tobac-co budworm, cotton bollworm, fall armyworm, beet armyworm, cabbage looper, soy-bean lopper and pink bollworm; the potato varieties “NewLeaf”, “NewLeaf Y” and “NewLeaf Plus” (Monsanto) with tobacco horn-worm resistance and the eggplant varie-ties “Bt brinjal”, “Dumaguete Long Purple”, “Mara” with resistance against brinjal fruit and shoot borer, bruit borer and cotton bollworm (see e.g. U.S. Pat. No. 5,128,130). Further trans-genic plants with insecticide resistance are commonly known, such as yellow stem-borer resistant rice (see e.g. Molecular Breeding, Volume 18, 2006, Number 1), lepi-dopteran resistant lettuce (see e.g. U.S. Pat. No. 5,349,124), resistant soybean (see e.g. U.S. Pat. No. 7,432,421) and rice with resistance against Lepidopterans, such as rice stemborer, rice skipper, rice cutworm, rice caseworm, rice leaffolder and rice armyworm (see e.g. WO2001021821). The methods of producing such transgenic plants are generally known to the person skilled in the art and are described, for example, in the publications mentioned above.

Preferably, plants, which are capable of synthesising antipathogenic substances are selected from soybean, tomatoes and cereals such as wheat, barley, rye and oat, most preferably from soybean and cereals such as wheat, barley, rye and oat.

Plants, which are capable of synthesising antipathogenic substances having a selec-tive action are for example plants expressing the so-called “pathogenesis-related pro-teins” (PRPs, see e.g. EP-A-0 392 225) or so-called “antifungal proteins” (AFPs, see e.g. U.S. Pat. No. 6,864,068). A wide range of antifungal proteins with activity against plant patho-genic fungi have been isolated from certain plant species and are common knowledge. Examples of such antipathogenic substances and transgenic plants capable of synthe-sising such antipathogenic substances are known, for example, from EP-A-0 392 225, WO93/05153, WO 95/33818, and EP-A-0 353 191. Transgenic plants which are re-sistant against fungicidal, viral and bacterial pathogens are produced by introducing plant resistance genes. Numerous resistant genes have been identified, isolated and were used to improve plant resistant, such as the N gene which was intro-duced into tobacco lines that are susceptible to Tobacco Mosaic Virus (TMV) in order to produce TMV-resistant tobacco plants (see e.g. U.S. Pat. No. 5,571,706), the Prf gene, which was intro-duced into plants to obtain enhanced pathogen resistance (see e.g. WO 199802545) and the Rps2 gene from Arabidopsis thaliana, which was used to create resistance to bacterial pathogens including Pseudomonas syringae (see e.g. WO 199528423). Plants exhibiting systemic acquired resistance response were obtained by introducing a nucleic acid molecule encoding the TIR domain of the N gene (see e.g. U.S. Pat. No. 6,630,618). Further examples of known resistance genes are the Xa21 gene, which has been introduced into a number of rice cultivars (see e.g. U.S. Pat. No. 5,952,485, U.S. Pat. No. 5,977,434, WO1999/09151, WO1996/22375), the Rcg1 gene for colletotrichum resistance (see e.g. US2006/225152), the prp1 gene (see e.g. U.S. Pat. No. 5,859,332, WO 2008017706), the ppv-cp gene to introduce resistance against plum pox virus (see e.g. US PP15,154Ps), the P1 gene (see e.g. U.S. Pat. No. 5,968,828), genes such as Blb1, Blb2, Blb3 and RB2 to introduce resistance against phytophthora infestans in potato (see e.g. U.S. Pat. No. 7,148,397), the LRPKmI gene (see e.g. WO1999064600), the P1 gene for potato virus Y resistance (see e.g. U.S. Pat. No. 5,968,828), the HA5-1 gene (see e.g. U.S. Pat. No. 5,877,403 and U.S. Pat. No. 6,046,384), the PIP gene to introduce a broad resistant to viruses, such as potato virus X (PVX), potato virus Y (PVY), potato leafroll virus (PLRV) (see e.g. EP0707069) and genes such as Arabidop-sis NI16, ScaM4 and ScaM5 genes to obtain fungicidal resistance (see e.g. U.S. Pat. No. 6,706,952 and EP1018553). The methods of producing such transgenic plants are generally known to the person skilled in the art and are described, for example, in the publications mentioned above.

Antipathogenic substances which can be expressed by such transgenic plants include, for example, ion channel blockers, such as blockers for sodium and calcium channels, for example the viral KP1, KP4 or KP6 toxins; stilbene synthases; bibenzyl synthases; chitinases; glucanases; the so-called “pathogenesis-related proteins”” (PRPs; see e.g. EP-A-0 392 225); antipathogenic substances produced by microorganisms, for exam-ple peptide antibiotics or heterocyclic antibiotics (see e.g. WO 95/33818) or protein or polypeptide factors involved in plant pathogen defense (so-called ““plant disease re-sistance genes””, as described in WO 03/000906).

Antipatogenic substances produced by the plants are able to protect the plants against a variety of pathogens, such as fungi, viruses and bacteria. Useful plants of elevated interest in connection with present invention are cereals, such as wheat, barley, rye and oat; soybean; maize; rice; oil seed rape; pome fruits; stone fruits; peanuts; coffee; tea; strawberries; turf; vines and vegetables, such as tomatoes, potatoes, cucurbits, papaya, melon, lenses and lettuce, more preferably selected from soybean, tomatoes and cereals such as wheat, barley, rye and oat, most preferably from soybean and cereals such as wheat, barley, rye and oat.

Transgenic plants with resistance against fungal pathogens, are, for examples, soy-beans with resistance against asian soybean rust (see e.g. WO 2008017706); plants such as alfalfa, corn, cotton, sugar beet, oileed, rape, tomato, soybean, wheat, potato and tobacco with resistance against phytophtora infestants (see e.g. U.S. Pat. No. 5,859,332, U.S. Pat. No. 7,148,397, EP1334979); corn with resistance against leaf blights, ear rots and stalk rots (such as anthracnose leaf bligh, anthracnose stalk rot, diplodia ear rot, fusarium verticilioides, gibberella zeae and top dieback, see e.g. US2006/225152); apples with resistance against apple scab (venturia inaequalis, see e.g. WO1999064600); plants such as rice, wheat, barley, rye, corn, oats, potato, melon, soybean and sorghum with resistance against fursarium diseases, such as fusarium graminearum, fusarium spo-rotrichioides, fusarium lateritium, fusarium pseudograminearum fusarium sambucinum, fusarium culmorum, fusarium poae, fusarium acuminatum, fusarium equiseti (see e.g. U.S. Pat. No. 6,646,184, EP1477557); plants, such as corn, soybean, cereals (in particular wheat, rye, barley, oats, rye, rice), tobacco, sorghum, sugarcane and potatoes with broad fun-gicidal resistance (see e.g. U.S. Pat. No. 5,689,046, U.S. Pat. No. 6,706,952, EP1018553 and U.S. Pat. No. 6,020,129).

Transgenic plants with resistance against bacterial pathogens and which are covered by the present invention, are, for examples, rice with resistance against xylella fastidi-osa (see e.g. U.S. Pat. No. 6,232,528); plants, such as rice, cotton, soybean, potato, sorghum, corn, wheat, barley, sugarcane, tomato and pepper, with resistance against bacterial blight (see e.g. WO2006/42145, U.S. Pat. No. 5,952,485, U.S. Pat. No. 5,977,434, WO1999/09151, WO1996/22375); tomato with resistance against pseudomonas syringae (see e.g. Can. J. Plant Path., 1983, 5: 251-255).

Transgenic plants with resistance against viral pathogens, are, for examples, stone fruits, such as plum, almond, apricot, cherry, peach, nectarine, with resistance against plum pox virus (PPV, see e.g. US PP15,154Ps, EP0626449); potatoes with resistance against potato virus Y (see e.g. U.S. Pat. No. 5,968,828); plants such as potato, tomato, cucumber and leguminosaes which are resistant against tomato spotted wilt virus (TSWV, see e.g. EP0626449, U.S. Pat. No. 5,973,135); corn with resistance against maize streak virus (see e.g. U.S. Pat. No. 6,040,496); papaya with resistance against papaya ring spot virus (PRSV, see e.g. S5877403, U.S. Pat. No. 6,046,384); cucurbitaceae, such as cucumber, melon, watermelon and pumpkin, and solanaceae, such as potato, tobacco, tomato, eggplant, paprika and pepper, with resistance against cucumber mosaic virus (CMV, see e.g. U.S. Pat. No. 6,849,780); cucurbitaceae, such as cucumber, melon, watermelon and pumpkin, with resistance against watermelon mosaic virus and zucchini yellow mosaic virus (see e.g. U.S. Pat. No. 6,015,942); potatoes with resistance against potato leafroll virus (PLRV, see e.g. U.S. Pat. No. 5,576,202); potatoes with a broad resistance to viruses, such as potato virus X (PVX), potato virus Y (PVY), potato leafroll virus (PLRV) (see e.g. EP0707069).

Plants which are resistant to antibiotics, such as kanamycin, neomycin and ampicillin. The naturally occurring bacterial nptII gene expresses the enzyme that blocks the ef-fects of the antibiotics kanamycin and neomycin. The ampicillin resistance gene ampR (also known as blaTEM1) is derived from the bacterium Salmonella paratyphi and is used as a marker gene in the transformation of micro-organisms and plants. It is re-sponsible for the synthesis of the enzyme betalactamase, which neutralises antibiotics in the penicillin group, including ampicillin. Transgenic plants with resistance against antibiotics, are, for examples potatoe, tomato, flax, canola, oilseed rape, rape seed and corn (see e.g. Plant Cell Reports, 20, 2001, 610-615. Trends in Plant Science, 11, 2006, 317-319. Plant Molecular Biology, 37, 1998, 287-296. Mol Gen Genet., 257, 1998, 606-13). Plant Cell Reports, 6, 1987, 333-336. Federal Register (USA), Vol. 60, No. 113, 1995, page 31139. Federal Register (USA), Vol. 67, No. 226, 2002, page 70392. Federal Register (USA), Vol. 63, No. 88, 1998, page 25194. Federal Register (USA), Vol. 60, No. 141, 1995, page 37870. Canadian Food Inspection Agency, FD/OFB-095-264-A, October 1999, FD/OFB-099-127-A, October 1999. Preferably, the plant is selected from soybean, tomatoes and cereals, such as wheat, barley, rye and oat, most preferably from soybean and cereals such as wheat, barley, rye and oat.

Plants which are tolerant to stress conditions (see e.g. WO 200004173, WO2007131699, CA2521729 and US20080229448) are plants, which show increased tolerance to abiotic stress conditions such as drought, high salinity, high light intensi-ties, high UV irradiation, chemical pollution (such as high heavy metal concentration), low or high temperatures, limited supply of nutrients (i.e. nitrogen, phosphorous) and population stress. Preferably, transgenic plants with resistance to stress conditions, are selected from rice, corn, soybean, sugarcane, alfalfa, wheat, tomato, potato, barley, rapeseed, beans, oats, sorghum and cotton with tolerance to drought (see e.g. WO2005048693, WO2008002480 and WO 2007030001); corn, soybean, wheat, cotton, rice, rapeseed and alfalfa with tolerance to low temperatures (see e.g. U.S. Pat. No. 4,731,499 and WO2007112122); rice, cotton, potato, soybean, wheat, barley, rye, sorghum, alfalfa, grape, tomato, sunflower and tobacco with tolerance to high salinity (see e.g. U.S. Pat. No. 7,256,326, U.S. Pat. No. 7,034,139, WO/2001/030990). The methods of producing such transgen-ic plants are generally known to the person skilled in the art and are described, for ex-ample, in the publications mentioned above. Preferably, the plant is selected from soy-bean, tomatoes and cereals such as wheat, barley, rye and oat, most preferably from soybean and cereals such as wheat, barley, rye and oat.

Altered maturation properties, are for example delayed ripening, delayed softening and early maturity. Preferably, transgenic plants with modified maturation properties, are, selected from tomato, melon, raspberry, strawberry, muskmelon, pepper and papaya with delayed ripening (see e.g. U.S. Pat. No. 5,767,376, U.S. Pat. No. 7,084,321, U.S. Pat. No. 6,107,548, U.S. Pat. No. 5,981,831, WO1995035387, U.S. Pat. No. 5,952,546, U.S. Pat. No. 5,512,466, WO1997001952, wo1992/008798, Plant Cell. 1989, 53-63. Plant Molecular Biology, 50, 2002). The methods of producing such transgenic plants are generally known to the person skilled in the art and are described, for example, in the publications mentioned above. Preferably, the plant is selected from fruits, such as tomato, vine, melon, papaya, banana, pepper, raspberry and strawberry; stone fruits, such as cherry, apricot and peach; pome fruits, such as apple and pear; and citrus fruits, such as citron, lime, orange, pomelo, grapefruit, and mandarin, more preferably from tomato, vine, apple, banana, orange and strawberry, most preferably tomatoes.

Content modification is synthesis of modified chemical compounds (if compared to the corresponding wildtype plant) or synthesis of enhanced amounts of chemical (if com-pounds compared to the corresponding wildtype plant) and corresponds to an in-creased or reduced amount of vitamins, amino acids, proteins and starch, different oils and a reduced amount of nicotine. Commercial examples are the soybean varieties “Vistive II” and “Visitive Ill” with low-linolenic/medium oleic content; the corn variety “Mavera high-value corn” with in-creased lysine content; and the soybean variety “Mavera high value soybean” with yielding 5% more protein compared to conventional varieties when processed into soy-bean meal. Further transgenic plants with altered content are, for example, potato and corn with modified amylopectin content (see e.g. U.S. Pat. No. 6,784,338, US20070261136); cano-la, corn, cotton, grape, catalpa, cattail, rice, soybean, wheat, sunflower, balsam pear and vernonia with a modified oil content (see e.g. U.S. Pat. No. 7,294,759, U.S. Pat. No. 7,157,621, U.S. Pat. No. 5,850,026, U.S. Pat. No. 6,441,278, U.S. Pat. Nos. 6,380,462, 6,365,802, U.S. Pat. No. 6,974,898, WO2001079499, US 20060075515 and U.S. Pat. No. 7,294,759); sunflower with increased fatty acid content (see e.g. U.S. Pat. No. 6,084,164); soybeans with modified allergens content (so called “hypoallergenic soy-bean, see e.g. U.S. Pat. No. 6,864,362); tobacco with reduced nicotine content (see e.g. US20060185684, WO2005000352 and WO2007064636); canola and soybean with increased lysine content (see e.g. Bio/Technology 13, 1995, 577-582); corn and soy-bean with altered composition of methionine, leucine, isoleucine and valine (see e.g. U.S. Pat. No. 6,946,589, U.S. Pat. No. 6,905,877); soybean with enhanced sulfur amino acid content (see e.g. EP0929685, WO1997041239); tomato with increased free amino acid contents, such as asparagine, aspartic acid, serine, threonine, alanine, histidine and glutamic acid (see e.g. U.S. Pat. No. 6,727,411); corn with enhanced amino acid content (see e.g. WO05077117); potato, corn and rice with modified starch content (see e.g. WO1997044471 and U.S. Pat. No. 7,317,146); tomato, corn, grape, alfalfa, apple, beans and peas with modified flavonoid content (see e.g. WO0004175); corn, rice, sorghum, cotton, soybeans with altered content of phenolic compounds (see e.g. US20080235829). The methods of producing such transgenic plants are generally known to the person skilled in the art and are described, for example, in the publications mentioned above. Preferably, the plant is selected from soybean, tomatoes and cereals such as wheat, barley, rye and oat, most preferably from soybean and cereals such as wheat, barley, rye and oat.

Enhanced nutrient utilization is e.g. assimilation or metabolism of nitrogen or phospho-rous. Preferably, transgenic plants with enhanced nitrogen assimilatory and utilization capacities are selected from for example, canola, corn, wheat, sunflower, rice, tobacco, soybean, cotton, alfalfa, tomato, wheat, potato, sugar beet, sugar cane and rapeseed (see e.g. WO1995009911, WO1997030163, U.S. Pat. No. 6,084,153, U.S. Pat. No. 5,955,651 and U.S. Pat. No. 6,864,405). Plants with improved phosphorous uptake are, for example, tomato and potatoe (see e.g. U.S. Pat. No. 7,417,181). The methods of producing such transgenic plants are generally known to the person skilled in the art and are described, for example, in the publications mentioned above. Preferably, the plant is selected from soybean, toma-toes and cereals such as wheat, barley, rye and oat, most preferably from soybean and cereals such as wheat, barley,

Transgenic plants with male steriliy are preferably selected from canola, corn, tomato, rice, Indian mustard, wheat, soybean and sunflower (see e.g. U.S. Pat. No. 6,720,481, U.S. Pat. No. 6,281,348, U.S. Pat. No. 5,659,124, U.S. Pat. No. 6,399,856, U.S. Pat. No. 7,345,222, U.S. Pat. No. 7,230,168, U.S. Pat. No. 6,072,102, EP1135982, WO2001092544 and WO1996040949). The methods of producing such transgenic plants are generally known to the person skilled in the art and are described, for example, in the publications mentioned above. Preferably, the plant is selected from soybean, tomatoes and cereals such as wheat, most preferably from soybean and cereals such as wheat, barley.

Plants, which produce higher quality fiber are e.g. transgenic cotton plants. The such improved quality of the fiber is related to improved micronaire of the fiber, increased strength, improved staple length, improved length uniformity and color of the fibers (see e.g. WO 1996/26639, U.S. Pat. No. 7,329,802, U.S. Pat. No. 6,472,588 and WO 2001/17333). The methods of producing such transgenic plants are generally known to the person skilled in the art and are described, for example, in the publications mentioned above.

As set forth above, cultivated plants may comprise one or more traits, e.g. selected from the group consisting of herbicide tolerance, insecticide resistance, fungicidal re-sistance, viral re-sistance, bacterial resistance, stress tolerance, maturation alteration, content modification, modified nutrient uptake and male sterility (see e.g. WO2005033319 and U.S. Pat. No. 6,376,754).

Examples of commercial available transgenic plants with two combined properties are the corn varieties “YieldGard Roundup Ready” and YieldGard Roundup Ready 2” (Monsanto) with glyphosate tolerance and resistance to corn borer; the corn variety “Agrisure CB/LL” (Syntenta) with glufosinate tolerance and corn borer resistance; the corn variety “Yield Gard VT Rootworm/RR2” with glyphosate tolerance and corn root-worm resistance; the corn variety “Yield Gard VT Triple” with glyphosate tolerance and resistance against corn rootworm and corn borer; the corn variety “Herculex I” with glufosinate tolerance and lepidopteran resistance (Cry1F), i.e. against western bean cutworm, corn borer, black cutworm and fall armyworm; the corn variety “YieldGard Corn Rootworm/Roundup Ready 2” (Monsanto) with glyphosate tolerance and corn rootworm resistance; the corn variety “Agrisure GT/RW” (Syngenta) with gluphosinate tolerance and lepidopteran resistance (Cry3A), i.e. against western corn rootworm, northern corn rootworm and Mexican corn rootworm; the corn variety “Herculex RW” (Dow, Pioneer) with glufosinate tolerance and lepidopteran resistance (Cry34/35Ab1), i.e. against western corn rootworm, northern corn rootworm and Mexican corn root-worm; the corn variety “Yield Gard VT Rootworm/RR2” with glyphosate tolerance and corn rootworm resistance; the soybean variety “Optimum GAT” (DuPont, Pioneer) with glyphosate tolerance and ALS herbicide tolerance; the corn variety “Mavera high-value corn” with glyphosate tolerance, resistance to corn rootworm and European corn borer and high lysine trait.

Examples of commercial available transgenic plants with three traits are the corn varie-ty “Herculex I/Roundup Ready 2” with glyphosate tolerance, gluphosinate tolerance and lepidopteran resistance (Cry1F), i.e. against western bean cutworm, corn borer, black cutworm and fall armyworm; the corn variety “YieldGard Plus/Roundup Ready 2” (Monsanto) with glyphosate tolerance, corn rootworm resistance and corn borer re-sistance; the corn variety “Agrisure GT/CB/LL” (Syngenta) with tolerance to glyphosate tolerance, tolerance to gluphosinate and corn borer resistance; the corn variety “Hercu-lex Xtra” (Dow, Pioneer) with glufosinate tolerance and lepidopteran resistance (Cry1F+Cry34/35Ab1), i.e. against western corn rootworm, northern corn rootworm, Mexican corn rootworm, western bean cutworm, corn borer, black cutworm and fall armyworm; the corn varieties “Agrisure CB/LL/RW” (Syngenta) with glufosinate toler-ance, corn borer resistance (Cry1Ab) and lepidopteran resistance (Cry3A), i.e. against western corn rootworm, northern corn rootworm and Mexican corn rootworm; the corn variety “Agrisure 3000GT” (Syngenta) with glyphosate tolerance+corn borer resistance (Cry1Ab) and lepidopteran resistance (Cry3A), i.e. against western corn rootworm, northern corn rootworm and Mexican corn rootworm. The methods of producing such transgenic plants are generally known to the person skilled in the art.

An example of a commercial available transgenic plant with four traits is “Hercules Quad-Stack” with glyphosate tolerance, glufosinate tolerance, corn borer resistance and corn rootworm resistance.

Preferably, the cultivated plants are plants, which comprise at least one trait selected from herbicide tolerance, insecticide resistance by expression of bacertial toxins, fungi-cidal resistance or viral resistance or bacterial resistance by expression of antipatho-genic substances, stress tolerance, content modification of chemicals present in the cultivated plant compared to the corresponding wild-type plant.

More preferably, the cultivated plants are plants, which comprise at least one trait se-lected from herbicide tolerance, insecticide resistance by expression of bacertial toxins, fungicidal resistance or viral resistance or bacterial resistance by expression of anti-pathogenic substances, content modification of chemicals present in the cultivated plant compared to the corresponding wild-type plant.

Most preferably, the cultivated plants are plants, which are tolerant to the action of herbicides and plants, which express bacterial toxins, which provides resistance against animal pests (such as insects or arachnids or nematodes), wherein the bacteri-al toxin is preferably a toxin from Bacillus thuriginensis. Herein, the cultivated plant is preferably selected from soybean, tomatoes and cereals such as wheat, barley, rye and oat, most preferably from soybean and cereals such as wheat, barley, rye and oat.

### Preferences

In one preferred method of soil application techniques, the active compound(s) are applied by drenching the soil.

In one preferred method of soil application techniques, the active compound(s) are applied by drip irrigation.

In one preferred method of soil application techniques, the active compound(s) are applied by soil injection.

In one preferred method of soil application techniques, the active compound(s) are applied by dipping roots, tubers or bulbs.

In one preferred method of soil application techniques, the active compound(s) are applied with drip application systems.

### Pests

The invention in particular relates to soil application methods for combating soil-living arthropod pests, and nematode pests, which comprises applying to the soil a pesticidally effective amount of a compound of the present invention.

The term “soil-living” means that the habitat, breeding ground, area or environment in which a pest or parasite is growing or may grow is the soil.

As stated above, in seed and soil treatment, there are certain pests which represent a big threat to plants during the stage from shoot/seedling to a small plant. There are pests which represent a threat, because they cause damage to plant roots, bulbs etc. There are pests which represent a threat, because, although they do not cause damage to roots and the such, they are merely developing in the soil so they can once again rise and become above-ground phytophagous or plant-eating pests. The soil-living pests especially include Coleoptera, which are beetles; Lepidoptera, which are moths and butterflies; Diptera, which are flies (especially Lycoriella, Sciara, Bradysia spp.); leafminers, cutworms, caterpillars, fungus gnats, mushroom flies, shore flies, black vine, carrot and strawberry-root weevils; sod webworms, wire- and potato-tuber worms; apple, carrot-rust fly, onion and cabbage maggots; and flea, June/May and cucumber beetle larvae.

Some pests which are especially known to represent a risk for the shoot/seedling or small plant, include rootworms, wireworms (e.g. in potatoe crop protection) and maggots like seedcorn maggot (e.g. Delia platura), western corn rootworm, black cutworm, mites, spider mites. These are only some examples; there are more pests specifically interesting in seed and soil treatment, which the person skilled in the art knows.

The use of the compounds according to the present invention extends to a wide range of different animal pests, especially soil living pests. The pests to be treated include but are not limited to, the following families, which include soil living pests:

insects from the order of the lepidopterans (Lepidoptera), for example Acronicta major, Adoxophyes orana, Aedia leucomelas, Agrotis spp. such as Agrotis fucosa, Agrotis segetum, Agrotis ypsilon; Alabama argillacea, Anticarsia gemmatalis, Anticarsia spp., Argyresthia conjugella, Autographa gamma, Barathra brassicae, Bucculatrix thurberiella, Bupalus piniarius, Cacoecia murinana, Cacoecia podana, Capua reticulana, Carpocapsa pomonella, Cheimatobia brumata, Chilo spp. such as Chilo suppressalis; Choristoneura fumiferana, Choristoneura occidentalis, Cirphis unipuncta, Clysia ambiguella, Cnaphalocerus spp., Cydia pomonella, Dendrolimus pini, Diaphania nitidalis, Diatraea grandiosella, Earias insulana, Elasmopalpus lignosellus, Ephestia cautella, Ephestia kuehniella, Eupoecilia ambiguella, Euproctis chrysorrhoea, Euxoa spp., Evetria bouliana, Feltia spp. such as Feltia subterranean; Galleria mellonella, Grapholitha funebrana, Grapholitha molesta, Helicoverpa spp. such as Helicoverpa armigera, Helicoverpa zea; Heliothis spp. such as Heliothis armigera, Heliothis virescens, Heliothis zea; Hellula undalis, Hibernia defoliaria, Hofmannophila pseudospretella, Homona magnanima, Hyphantria cunea, Hyponomeuta padella, Hyponomeuta malinellus, Keiferia lycopersicella, Lambdina fiscellaria, Laphygma spp. such as Laphygma exigua; Leucoptera coffeella, Leucoptera scitella, Lithocolletis blancardella, Lithophane antennata, Lobesia botrana, Loxagrotis albicosta, Loxostege sticticalis, Lymantria spp. such as Lymantria dispar, Lymantria monacha; Lyonetia clerkella, Malacosoma neustria, Mamestra spp. such as Mamestra brassicae; Mocis repanda, Mythimna separata, Orgyia pseudotsugata, Oria spp., Ostrinia spp. such as Ostrinia nubilalis; Oulema oryzae, Panolis flammea, Pectinophora spp. such as Pectinophora gossypiella; Peridroma saucia, Phalera bucephala, Phthorimaea spp. such as Phthorimaea operculella; Phyllocnistis citrella, Pieris spp. such as Pieris brassicae, Pieris rapae; Plathypena scabra, Plutella maculipennis, Plutella xylostella, Prodenia spp., Pseudaletia spp., Pseudoplusia includens, Pyrausta nubilalis, Rhyacionia frustrana, Scrobipalpula absoluta, Sitotroga cerealella, Sparganothis pilleriana, Spodoptera spp. such as Spodoptera frugiperda, Spodoptera littoralis, Spodoptera litura; Thaumatopoea pityocampa, Thermesia gemmatalis, Tinea pellionella, Tineola bisselliella, Tortrix viridana, Trichoplusia spp. such as Trichoplusia ni; Tuta absoluta, and Zeiraphera canadensis,
beetles (Coleoptera), for example Acanthoscehdes obtectus, Adoretus spp., Agelastica alni, Agrilus sinuatus, Agriotes spp. such as Agriotes fuscicollis, Agriotes lineatus, Agriotes obscurus; Amphimallus solstitialis, Anisandrus dispar, Anobium punctatum, Anomala rufocuprea, Anoplophora spp. such as Anoplophora glabripennis; Anthonomus spp. such as Anthonomus grandis, Anthonomus pomorum; Anthrenus spp., Aphthona euphoridae, Apogonia spp., Athous haemorrhoidalis, Atomaria spp. such as Atomaria linearis; Attagenus spp., Aulacophora femoralis, Blastophagus piniperda, Blitophaga undata, Bruchidius obtectus, Bruchus spp. such as Bruchus lentis, Bruchus pisorum, Bruchus rufimanus; Byctiscus betulae, Callosobruchus chinensis, Cassida nebulosa, Cerotoma trifurcata, Cetonia aurata, Ceuthorhynchus spp. such as Ceuthorrhynchus assimilis, Ceuthorrhynchus napi; Chaetocnema tibialis, Cleonus mendicus, Conoderus spp. such as Conoderus vespertinus; Cosmopolites spp., Costelytra zealandica, Crioceris asparagi, Cryptorhynchus lapathi, Ctenicera ssp. such as Ctenicera destructor; Curculio spp., Dectes texanus, Dermestes spp., Diabrotica spp. such as Diabrotica 12-punctata Diabrotica speciosa, Diabrotica longicornis, Diabrotica semipunctata, Diabrotica virgifera; Epilachna spp. such as Epilachna varivestis, Epilachna vigintioctomaculata; Epitrix spp. such as Epitrix hirtipennis; Eutinobothrus brasiliensis, Faustinus cubae, Gibbium psylloides, Heteronychus arator, Hylamorpha elegans, Hylobius abietis, Hylotrupes bajulus, Hypera brunneipennis, Hypera postica, Hypothenemus spp., Ips typographus, Lachnosterna consanguinea, Lema bilineata, Lema melanopus, Leptinotarsa spp. such as Leptinotarsa decemlineata; Limonius californicus, Lissorhoptrus oryzophilus, Lissorhoptrus oryzophilus, Lixus spp., Lyctus spp. such as Lyctus bruneus; Melanotus communis, Meligethes spp. such as Meligethes aeneus; Melolontha hippocastani, Melolontha melolontha, Migdolus spp., Monochamus spp. such as Monochamus alternatus; Naupactus xanthographus, Niptus hololeucus, Oryctes rhinoceros, Oryzaephilus surinamensis, Otiorrhynchus sulcatus, Otiorrhynchus ovatus, Otiorrhynchus sulcatus, Oulema oryzae, Oxycetonia jucunda, Phaedon cochleariae, Phyllobius pyri, Phyllopertha horticola, Phyllophaga spp., Phyllotreta spp. such as Phyllotreta chrysocephala, Phyllotreta nemorum, Phyllotreta striolata; Phyllophaga spp., Phyllopertha horticola, Popillia japonica, Premnotrypes spp., Psylliodes chrysocephala, Ptinus spp., Rhizobius ventralis, Rhizopertha dominica, Sitona lineatus, Sitophilus spp. such as Sitophilus granaria, Sitophilus zeamais; Sphenophorus spp. such as Sphenophorus levis; Sternechus spp. such as Sternechus subsignatus; Symphyletes spp., Tenebrio molitor, Tribolium spp. such as Tribolium castaneum; Trogoderma spp., Tychius spp., Xylotrechus spp., and Zabrus spp. such as Zabrus tenebrioides,
flies, mosquitoes (Diptera), e.g. Aedes spp. such as Aedes aegypti, Aedes albopictus, Aedes vexans; Anastrepha ludens, Anopheles spp. such as Anopheles albimanus, Anopheles crucians, Anopheles freeborni, Anopheles gambiae, Anopheles leucosphyrus, Anopheles maculipennis, Anopheles minimus, Anopheles quadrimaculatus, Anopheles sinensis; Bibio hortulanus, Calliphora erythrocephala, Calliphora vicina, Cerafitis capitata, Ceratitis capitata, Chrysomyia spp. such as Chrysomya bezziana, Chrysomya hominivorax, Chrysomya macellaria; Chrysops atlanticus, Chrysops discalis, Chrysops silacea, Cochliomyia spp. such as Cochliomyia hominivorax; Contarinia spp. such as Contarinia sorghicola; Cordylobia anthropophaga, Culex spp. such as Culex nigripalpus, Culex pipiens, Culex quinquefasciatus, Culex tarsalis, Culex tritaeniorhynchus; Culicoides furens, Culiseta inornata, Culiseta melanura, Cuterebra spp., Dacus cucurbitae, Dacus oleae, Dasineura brassicae, Delia spp. such as Delia antique, Delia coarctata, Delia platura, Delia radicum; Dermatobia hominis, Drosophila spp., Fannia spp. such as Fannia canicularis; Gastraphilus spp. such as Gasterophilus intestinalis; Geomyza Tripunctata, Glossina fuscipes, Glossina morsitans, Glossina palpalis, Glossina tachinoides, Haematobia irritans, Haplodiplosis equestris, Hippelates spp., Hylemyia spp. such as Hylemyia platura; Hypoderma spp. such as Hypoderma lineata; Hyppobosca spp., Leptoconops torrens, Liriomyza spp. such as Liriomyza sativae, Liriomyza trifolii; Lucilia spp. such as Lucilia caprina, Lucilia cuprina, Lucilia sericata; Lycoria pectoralis, Mansonia titillanus, Mayetiola spp. such as Mayetiola destructor; Musca spp. such as Musca autumnalis, Musca domestica; Muscina stabulans, Oestrus spp. such as Oestrus ovis; Opomyza florum, Oscinella spp. such as Oscinella frit; Pegomya hysocyami, Phlebotomus argentipes, Phorbia spp. such as Phorbia antiqua, Phorbia brassicae, Phorbia coarctata; Prosimulium mixtum, Psila rosae, Psorophora columbiae, Psorophora discolor, Rhagoletis cerasi, Rhagoletis pomonella, Sarcophaga spp. such as Sarcophaga haemorrhoidalis; Simulium vittatum, Stomoxys spp. such as Stomoxys calcitrans; Tabanus spp. such as Tabanus atratus, Tabanus bovinus, Tabanus lineola, Tabanus similis; Tannia spp., Tipula oleracea, Tipula paludosa, and Wohlfahrtia spp.,
thrips (Thysanoptera), e.g. Baliothrips biformis, Dichromothrips corbetti, Dichromothrips ssp., Enneothrips flavens, Frankliniella spp. such as Frankliniella fusca, Frankliniella occidentalis, Frankliniella tritici; Heliothrips spp., Hercinothrips femoralis, Kakothrips spp., Rhipiphorothrips cruentatus, Scirtothrips spp. such as Scirtothrips citri; Taeniothrips cardamoni, Thrips spp. such as Thrips oryzae, Thrips palmi, Thrips tabaci;
termites (Isoptera), e.g. Calotermes flavicollis, Coptotermes formosanus, Heterotermes aureus, Heterotermes longiceps, Heterotermes tenuis, Leucotermes flavipes, Odontotermes spp., Reticulitermes spp. such as Reticulitermes speratus, Reticulitermes flavipes, Reticulitermes grassei, Reticulitermes lucifugus, Reticulitermes santonensis, Reticulitermes virginicus; Termes natalensis,
cockroaches (Blattaria-Blattodea), e.g. Acheta domesticus, Blatta orientalis, Blattella asahinae, Blattella germanica, Gryllotalpa spp., Leucophaea maderae, Locusta spp., Melanoplus spp., Periplaneta americana, Periplaneta australasiae, Periplaneta brunnea, Periplaneta fuligginosa, Periplaneta japonica,
bugs, aphids, leafhoppers, whiteflies, scale insects, cicadas (Hemiptera), e.g. Acrosternum spp. such as Acrosternum hilare; Acyrthosipon spp. such as Acyrthosiphon onobrychis, Acyrthosiphon pisum; Adelges laricis, Aeneolamia spp., Agonoscena spp., Aleurodes spp., Aleurolobus barodensis, Aleurothrixus spp., Amrasca spp., Anasa tristis, Antestiopsis spp., Anuraphis cardui, Aonidiella spp., Aphanostigma piri, Aphidula nasturtii, Aphis spp. such as Aphis fabae, Aphis forbesi, Aphis gossypii, Aphis grossulariae, Aphis pomi, Aphis sambuci, Aphis schneideri, Aphis spiraecola; Arboridia apicalis, Arilus critatus, Aspidiella spp., Aspidiotus spp., Atanus spp., Aulacorthum solani, Bemisia spp. such as Bemisia argentifolii, Bemisia tabaci; Blissus spp. such as Blissus leucopterus; Brachycaudus cardui, Brachycaudus helichrysi, Brachycaudus persicae, Brachycaudus prunicola, Brachycolus spp., Brevicoryne brassicae, Calligypona marginata, Calocoris spp., Campylomma livida, Capitophorus horni, Carneocephala fulgida, Cavelerius spp., Ceraplastes spp., Ceratovacuna lanigera, Cercopidae, Cerosipha gossypii, Chaetosiphon fragaefolii, Chionaspis tegalensis, Chlorita onukii, Chromaphis juglandicola, Chrysomphalus ficus, Cicadulina mbila, Cimex spp. such as Cimex hemipterus, Cimex lectularius; Coccomytilus halli, Coccus spp., Creontiades dilutus, Cryptomyzus ribis, Cryptomyzus ribis, Cyrtopeltis notatus, Dalbulus spp., Dasynus piperis, Dialeurades spp., Diaphorina spp., Diaspis spp., Dichelops furcatus, Diconocoris hewetti, Doralis spp., Dreyfusia nordmannianae, Dreyfusia piceae, Drosicha spp., Dysaphis spp. such as Dysaphis plantaginea, Dysaphis pyri, Dysaphis radicola; Dysaulacorthum pseudosolani, Dysdercus spp. such as Dysdercus cingulatus, Dysdercus intermedius; Dysmicoccus spp., Empoasca spp. such as Empoasca fabae, Empoasca solana; Eriosoma spp., Erythroneura spp., Eurygaster spp. such as Eurygaster integriceps; Euscelis bilobatus, Euschistus spp. such as Euschistuos heros, Euschistus impictiventris, Euschistus servus; Geococcus coffeae, Halyomorpha spp. such as Halyomorpha halys; Heliopeltis spp., Homalodisca coagulata, Horcias nobilellus, Hyalopterus pruni, Hyperomyzus lactucae, Icerya spp., Idiocerus spp., Idioscopus spp., Laodelphax striatellus, Lecanium zspp., Lepidosaphes spp., Leptocorisa spp., Leptoglossus phyllopus, Lipaphis erysimi, Lygus spp. such as Lygus hesperus, Lygus lineolaris, Lygus pratensis; Macropes excavatus, Macrosiphum spp. such as Macrosiphum rosae, Macrosiphum avenae, Macrosiphum euphorbiae; Mahanarva fimbriolata, Megacopta cribraria, Megoura viciae, Melanaphis pyrarius, Melanaphis sacchari, Metcafiella spp., Metopolophium dirhodum, Miridae spp., Monellia costalis, Monelliopsis pecanis, Myzus spp. such as Myzus ascalonicus, Myzus cerasi, Myzus persicae, Myzus varians; Nasonovia ribis-nigri, Nephotettix spp. such as Nephotettix malayanus, Nephotettix nigropictus, Nephotettix parvus, Nephotettix virescens; Nezara spp. such as Nezara viridula; Nilaparvata lugens, Oebalus spp., Oncometopia spp., Orthezia praelonga, Parabemisia myricae, Paratrioza spp., Parlatoria spp., Pemphigus spp. such as Pemphigus bursarius; Pentomidae, Peregrinus maidis, Perkinsiella saccharicida, Phenacoccus spp., Phloeomyzus passerinii, Phorodon humuli, Phylloxera spp., Piesma quadrata, Piezodorus spp. such as Piezodorus guildinii, Pinnaspis aspidistrae, Planococcus spp., Protopulvinaria pyriformis, Psallus seriatus, Pseudacysta persea, Pseudaulacaspis pentagona, Pseudococcus spp. such as Pseudococcus comstocki; Psylla spp. such as Psylla mali, Psylla piri; Pteromalus spp., Pyrilla spp., Quadraspidiotus spp., Quesada gigas, Rastrococcus spp., Reduvius senilis, Rhodnius spp., Rhopalomyzus ascalonicus, Rhopalosiphum spp. such as Rhopalosiphum pseudobrassicas, Rhopalosiphum insertum, Rhopalosiphum maidis, Rhopalosiphum padi; Sagatodes spp., Sahlbergella singularis, Saissetia spp., Sappaphis mala, Sappaphis mali, Scaphoides titanus, Schizaphis graminum, Schizoneura lanuginosa, Scotinophora spp., Selenaspidus articulatus, Sitobion avenae, Sogata spp., Sogatella furcifera, Solubea insularis, Stephanitis nashi, Stictocephala festina, Tenalaphara malayensis, Thyanta spp. such as Thyanta perditor; Tibraca spp., Tinocallis caryaefoliae, Tomaspis spp., Toxoptera spp. such as Toxoptera aurantii; Trialeurodes spp. such as Trialeurodes vaporariorum; Triatoma spp., Trioza spp., Typhlocyba spp., Unaspis spp. such as Unaspis yanonensis; and Viteus vitifolii,
ants, bees, wasps, sawflies (Hymenoptera), e.g. Athalia rosae, Atta capiguara, Atta cephalotes, Atta cephalotes, Atta laevigata, Atta robusta, Atta sexdens, Atta texana, Bombus spp., Camponotus floridanus, Crematogaster spp., Dasymutilla occidentalis, Diprion spp., Dolichovespula maculata, Hoplocampa spp. such as Hoplocampa minuta, Hoplocampa testudinea; Lasius spp. such as Lasius niger, Linepithema humile, Monomorium pharaonis, Paravespula germanica, Paravespula pennsylvanica, Paravespula vulgaris, Pheidole megacephala, Pogonomyrmex barbatus, Pogonomyrmex californicus, Polistes rubiginosa, Solenopsis geminata, Solenopsis invicta, Solenopsis richteri, Solenopsis xyloni, Vespa spp. such as Vespa crabro, and Vespula squamosa,
crickets, grasshoppers, locusts (Orthoptera), e.g. Acheta domestica, Calliptamus italicus, Chortoicetes terminifera, Dociostaurus maroccanus, Gryllotalpa africana, Gryllotalpa gryllotalpa, Hieroglyphus daganensis, Kraussaria angulifera, Locusta migratoria, Locustana pardalina, Melanoplus bivittatus, Melanoplus femurrubrum, Melanoplus mexicanus, Melanoplus sanguinipes, Melanoplus spretus, Nomadacris septemfasciata, Oedaleus senegalensis, Schistocerca americana, Schistocerca gregaria, Tachycines asynamorus, and Zonozerus variegatus,
arachnids (Arachnida), such as acari, e.g. of the families Argasidae, Ixodidae and Sarcoptidae, such as Amblyomma spp. (e.g. Amblyomma americanum, Amblyomma variegatum, Amblyomma maculatum), Argas spp. (e.g. Argas persicus), Boophilus spp. (e.g. Boophilus annulatus, Boophilus decoloratus, Boophilus microplus), Dermacentor silvarum, Dermacentor andersoni, Dermacentor variabilis, Hyalomma spp. (e.g. Hyalomma truncatum), Ixodes spp. (e.g. Ixodes ricinus, Ixodes rubicundus, Ixodes scapularis, Ixodes holocyclus, Ixodes pacificus), Ornithodorus spp. (e.g. Ornithodorus moubata, Ornithodorus hermsi, Ornithodorus turicata), Ornithonyssus bacoti, Otobius megnini, Dermanyssus gallinae, Psoroptes spp. (e.g. Psoroptes ovis), Rhipicephalus spp. (e.g. Rhipicephalus sanguineus, Rhipicephalus appendiculatus, Rhipicephalus evertsi), Rhizoglyphus spp., Sarcoptes spp. (e.g. Sarcoptes scabiei), and Eriophyidae spp. such as Acaria sheldoni, Aculops spp. (e.g. Aculops pelekassi) Aculus spp. (e.g. Aculus schlechtendali), Epitrimerus pyri, Phyllocoptruta oleivora and Eriophyes spp. (e.g. Eriophyes sheldoni); Tarsonemidae spp. such as Hemitarsonemus spp., Phytonemus pallidus and Polyphagotarsonemus latus, Stenotarsonemus spp.; Tenuipalpidae spp. such as Brevipalpus spp. (e.g. Brevipalpus phoenicis); Tetranychidae spp. such as Eotetranychus spp., Eutetranychus spp., Oligonychus spp., Tetranychus cinnabarinus, Tetranychus kanzawai, Tetranychus pacificus, Tetranychus telarius and Tetranychus urticae; Bryobia praetiosa, Panonychus spp. (e.g. Panonychus ulmi, Panonychus citri), Metatetranychus spp. and Oligonychus spp. (e.g. Oligonychus pratensis), Vasates lycopersici; Araneida, e.g. Latrodectus mactans, and Loxosceles reclusa. And Acarus siro, Chorioptes spp., Scorpio maurus
fleas (Siphonaptera), e.g. Ceratophyllus spp., Ctenocephalides felis, Ctenocephalides canis, Xenopsylla cheopis, Pulex irritans, Tunga penetrans, and Nosopsyllus fasciatus,
silverfish, firebrat (Thysanura), e.g. Lepisma saccharina and Thermobia domestica, centipedes (Chilopoda), e.g. Geophilus spp., Scutigera spp. such as Scutigera coleoptrata;
millipedes (Diplopoda), e.g. Blaniulus guttulatus, Narceus spp.,
Earwigs (Dermaptera), e.g. forficula auricularia,
lice (Phthiraptera), e.g. Damalinia spp., Pediculus spp. such as Pediculus humanus capitis, Pediculus humanus corporis; Pthirus pubis, Haematopinus spp. such as Haematopinus eurysternus, Haematopinus suis; Linognathus spp. such as Linognathus vituli; Bovicola bovis, Menopon gallinae, Menacanthus stramineus and Solenopotes capillatus, Trichodectes spp.,
springtails (Collembola), e.g. Onychiurus ssp. such as Onychiurus armatus,

They are also suitable for controlling nematodes: plant parasitic nematodes such as root knot nematodes, Meloidogyne hapla, Meloidogyne incognita, Meloidogyne javanica, and other Meloidogyne species; cyst-forming nematodes, Globodera rostochiensis and other Globodera species; Heterodera avenae, Heterodera glycines, Heterodera schachtii, Heterodera trifolii, and other Heterodera species; Seed gall nematodes, Anguina species; Stem and foliar nematodes, Aphelenchoides species such as Aphelenchoides besseyi; Sting nematodes, Belonolaimus longicaudatus and other Belonolaimus species; Pine nematodes, Bursaphelenchus lignicolus Mamiya et Kiyohara, Bursaphelenchus xylophilus and other Bursaphelenchus species; Ring nematodes, Criconema species, Criconemella species, Criconemoides species, Mesocriconema species; Stem and bulb nematodes, Ditylenchus destructor, Ditylenchus dipsaci and other Ditylenchus species; Awl nematodes, Dolichodorus species; Spiral nematodes, Helicotylenchus multicinctus and other Helicotylenchus species; Sheath and sheathoid nematodes, Hemicycliophora species and Hemicriconemoides species; Hirshmanniella species; Lance nematodes, Hoploaimus species; false rootknot nematodes, Nacobbus species; Needle nematodes, Longidorus elongatus and other Longidorus species; Lesion nematodes, Pratylenchus brachyurus, Pratylenchus neglectus, Pratylenchus penetrans, Pratylenchus curvitatus, Pratylenchus goodeyi and other Pratylenchus species; Burrowing nematodes, Radopholus similis and other Radopholus species; Reniform nematodes, Rotylenchus robustus, Rotylenchus reniformis and other Rotylenchus species; Scutellonema species; Stubby root nematodes, Trichodorus primitivus and other Trichodorus species, Paratrichodorus species; Stunt nematodes, Tylenchorhynchus claytoni, Tylenchorhynchus dubius and other Tylenchorhynchus species; Citrus nematodes, Tylenchulus species such as Tylenchulus semipenetrans; Dagger nematodes, Xiphinema species; and other plant parasitic nematode species.

Examples of further pest species which may be controlled by compounds of formula (I) include: from the class of the Bivalva, for example, Dreissena spp.; from the class of the Gastropoda, for example, Arion spp., Biomphalaria spp., Bulinus spp., Deroceras spp., Galba spp., Lymnaea spp., Oncomelania spp., Succinea spp.; from the class of the helminths, for example, Ancylostoma duodenale, Ancylostoma ceylanicum, Acylostoma braziliensis, Ancylostoma spp., Ascaris lumbricoides, Ascaris spp., Brugia malayi, Brugia timori, Bunostomum spp., Chabertia spp., Clonorchis spp., Cooperia spp., Dicrocoelium spp., Dictyocaulus filaria, Diphyllobothrium latum, Dracunculus medinensis, Echinococcus granulosus, Echinococcus multilocularis, Enterobius vermicularis, Faciola spp., Haemonchus spp. such as Haemonchus contortus; Heterakis spp., Hymenolepis nana, Hyostrongulus spp., Loa Loa, Nematodirus spp., Oesophagostomum spp., Opisthorchis spp., Onchocerca volvulus, Ostertagia spp., Paragonimus spp., Schistosomen spp., Strongyloides fuelleborni, Strongyloides stercora lis, Stronyloides spp., Taenia saginata, Taenia solium, Trichinella spiralis, Trichinella nativa, Trichinella britovi, Trichinella nelsoni, Trichinella pseudopsiralis, Trichostrongulus spp., Trichuris trichiura, Wuchereria bancrofti; from the order of the Isopoda, for example, Armadillidium vulgare, Oniscus asellus, Porcellio scaber; from the order of the Symphyla, for example, Scutigerella immaculata.

Further examples of pest species which may be controlled by compounds of formula (I) include: Anisoplia austriaca, Apamea spp., Austroasca viridigrisea, Baliothrips biformis, Caenorhabditis elegans, Cephus spp., Ceutorhynchus napi, Chaetocnema aridula, Chilo auricilius, Chilo indicus, Chilo polychrysus, Chortiocetes terminifera, Cnaphalocroci medinalis, Cnaphalocrosis spp., Colias eurytheme, Collops spp., Cornitermes cumulans, Creontiades spp., Cyclocephala spp., Dalbulus maidis, Deraceras reticulatum, Diatrea saccharalis, Dichelops furcatus, Dicladispa armigera, Diloboderus spp. such as Diloboderus abderus; Edessa spp., Epinotia spp., Formicidae, Geocoris spp., Globitermes sulfureus, Gryllotalpidae, Halotydeus destructor, Hipnodes bicolor, Hydrellia philippina, Julus spp., Laodelphax spp., Leptocorsia acuta, Leptocorsia oratorius, Liogenys fuscus, Lucillia spp., Lyogenys fuscus, Mahanarva spp., Maladera matrida, Marasmia spp., Mastotermes spp., Mealybugs, Megascelis ssp, Metamasius hemipterus, Microtheca spp., Mocis latipes, Murgantia spp., Mythemina separata, Neocapritermes opacus, Neocapritermes parvus, Neomegalotomus spp., Neotermes spp., Nymphula depunctalis, Oebalus pugnax, Orseolia spp. such as Orseolia oryzae; Oxycaraenus hyalinipennis, Plusia spp., Pomacea canaliculata, Procornitermes ssp, Procornitermes triacifer, Psylloides spp., Rachiplusia spp., Rhodopholus spp., Scaptocoris castanea, Scaptocoris spp., Scirpophaga spp. such as Scirpophaga incertulas, Scirpophaga innotata; Scotinophara spp. such as Scotinophara coarctata; Sesamia spp. such as Sesamia inferens, Sogaella frucifera, Solenapsis geminata, Spissistilus spp., Stalk borer, Stenchaetothrips biformis, Steneotarsonemus spinki, Sylepta derogata, Telehin licus, Trichostrongylus spp.

Other animal pests, which are especially controlled and combated by the methods of the present invention are:

From the family of the Pemphigidae, preferably: Eriosoma spp., Pemphigus spp., Anuraphis spp., Brachycaudus spp., in crops such as, for example, pome fruit, conifers, vegetables and ornamentals.

From the psyllid family (Psyllidae), preferably: Psylla spp., Paratrioza spp., Trioza spp., in crops such as, for example, citrus, vegetables, potatoes, pome fruit.

From the scale insect family (Coccidae), preferably: Ceroplastes spp., Drosicha spp. Pulvinaria spp., Protopuhninaria spp., Saissetia spp., Coccus spp., in perennial crops such as, for example, citrus, grapevines, tea, pome and stone fruit, tropical crops, ornamentals, conifers, but also vegetables.

From the family of the Diaspididae, preferably: Quadraspidiotus spp., Aonidiella spp., Lepidosaphes spp., Aspidiotus spp., Aspis spp., Diaspis spp., Parlatoria spp., Pseudaulacaspis spp., Unaspis spp., Pinnaspis spp., Selenaspidus spp., in crops such as, for example, citrus, tea, ornamentals, conifers, pome and stone fruit, grapevines, tropical crops.

From the family of the Pseudococcidae, preferably: Pericerga, Pseudococcus spp., Planococcus spp., Phenacoccus spp., Dysmicoccus spp., in crops such as, for example, citrus, pome and stone fruit, tea, grapevines, vegetables, ornamentals, conifers, spices and tropical crops.

Furthermore from the family of the Aleyrodidae are preferably treated according to the methods of the present invention: Bemisia argentifolii, Bemisia tabaci, Trialeurodes vaporariorum, Aleurothrixus floccosus, Aleurodes spp., Dialeurodes spp., Parabemisia myricae in crops such as, for example, vegetables, melons, potatoes, tobacco, soft fruit, citrus, ornamentals, conifers, cotton, potatoes and tropical crops.

From the family of the Aphidae are preferably treated according to the methods of the present invention:

Myzus spp. in tobacco, stone fruit, pome fruit, soft fruit, Brassica vegetables, fruiting vegetables, leafy vegetables, tuber and root vegetables, melons, potatoes, spices, ornamentals and conifers.
Aphis spp. in cotton, tobacco, citrus, melons, beet, soft fruit, oilseed rape, fruiting vegetables, leafy vegetables, Brassica vegetables, tuber and root vegetables, ornamentals, potatoes, pumpkins, spices. Rhodobium porosum in strawberries,
Nasonovia ribisnigri in leafy vegetables,
Macrosiphum spp. in ornamentals, cereals, potatoes, leafy vegetables, Brassica vegetables and fruiting vegetables, strawberries, Phorodon humuli in hops, Toxoptera spp. in citrus, stone fruit, almonds, nuts, cereals, spices,
Aulacorthum spp. in citrus, potatoes, fruiting vegetables and leafy vegetables.

From the family of the Tetranychidae are preferably treated according to the methods of the present invention: Tetranychus spp., Brevipalpus spp., Panonychus spp., Oligonycbus spp., Eotetranychus spp., Bryobia spp. in crops such as, for example, vegetables, ornamentals, spices, conifers, citrus, stone and pome fruit, grapevines, cotton, soft fruit, melons, potatoes.

From the family of the Tarsonemidae are preferably treated according to the methods of the present invention: Hermitarsonernus batus, Stenotarsonemus spp., Polyphagotarsonemus spp., Stenotarsonemus spinkiin crops such as, for example, vegetables, ornamentals, spices, conifers, tea, citrus, melons.

From the thrips family (Thripidae) are preferably treated according to the methods of the present invention: Anaphothrips spp., Baliothrips spp., Caliothrips spp., Franklinella spp., Heliothrips spp., Hercrnothrips spp., Rhipiphorothrips spp., Scirtothrips spp., Selenothrips spp. and Thrips spp., in crops such as, for example, fruit, cotton, grapevines, soft fruit, vegetables, melons, ornamentals, spices, conifers, tropical crops, tea.

Also preferred species are the following from the whitefly family (Agromyzidae): Liriomyza spp., Pegomya spp. in crops such as, for example, vegetables, melons, potatoes and ornamentals.

Also preferred species are the following from the foliar nematode family (Aphelenchoididae), for example Aphelenchoides ritzemabosi, A. fragariae, A. besseyi, A. blastophthorus in crops such as soft fruits and ornamentals.

Most preferably the methods of the present invention are applied to control and combat arachnids, especially the following ones from the family of the Tetranychidae:

### Tetranychus spp., Brevipalpus spp., Panonychus spp., Oligonycbus spp., Eotetranychus spp. and Bryobia spp.

In a preferred embodiment, the methods and uses according to the invention are as follows:

TABLE AP-T

| Appl. | | | |
|---|---|---|---|
| type | | Pest | Crop |
| AP-T-1 | ST | Agrotis ipsilon | as defined herein |
| AP-T-2 | ST | Spodoptera | as defined herein |
| | | frugiperta | |
| AP-T-3 | ST | Phyllotreta sp. | as defined herein |
| AP-T-4 | ST | Stem Girdler | as defined herein |
| AP-T-5 | ST | Agriotes sp. | as defined herein |
| AP-T-6 | ST | Delia platura | as defined herein |
| AP-T-7 | ST | Agrotis ipsilon | Soybean |
| AP-T-8 | ST | Spodoptera | Soybean |
| | | frugiperta | |
| AP-T-9 | ST | Phyllotreta sp. | Soybean |
| AP-T-10 | ST | Stem Girdler | Soybean |
| AP-T-11 | ST | Agriotes sp. | Soybean |
| AP-T-12 | ST | Delia platura | Soybean |
| AP-T-13 | ST | Agrotis ipsilon | Corn |
| AP-T-14 | ST | Spodoptera | Corn |
| | | frugiperta | |
| AP-T-15 | ST | Phyllotreta sp. | Corn |
| AP-T-16 | ST | Stem Girdler | Corn |
| AP-T-17 | ST | Agriotes sp. | Corn |
| AP-T-18 | ST | Delia platura | Corn |

(Abbreviation: ST = seed treatment)

### Preferences

In an embodiment of the invention, the methods of the present invention are used for controlling pests from the family Aphididae.

In an embodiment of the invention, the methods of the present invention are used for controlling pests from the family Phemphigidae.

In an embodiment of the invention, the methods of the present invention are used for controlling pests from the family Phemphigidae.

In an embodiment of the invention, the methods of the present invention are used for controlling pests from the family Tetranychidae.

In an embodiment of the invention, the methods of the present invention are used for controlling pests from the family Tarsonemidae.

In an embodiment of the invention, the methods of the present invention are used for controlling pests from the family Thripidae.

In an embodiment of the invention, the methods of the present invention are used for controlling pests from the family Aleyrodidae

In an embodiment of the invention, the methods of the present invention are used for controlling pests from the family Coccidae.

In an embodiment of the invention, the methods of the present invention are used for controlling pests from the family Pseudococcidae.

In an embodiment of the invention, the methods of the present invention are used for controlling pests from the family Agromyzidae.

In an embodiment of the invention, the methods of the present invention are used for controlling pests from the family Aphelenchoididae.

In a preferred embodiment of the invention, the methods of the present invention are used for controlling pests from the family Coleoptera.

In a preferred embodiment of the invention, the methods of the present invention are used for controlling pests from the family Lepidoptera.

In a preferred embodiment of the invention, the methods of the present invention are used for controlling pests from the family Orthoptera.

In a preferred embodiment of the invention, the methods of the present invention are used for controlling pests from the family Hemiptera.

In a preferred embodiment of the invention, the methods of the present invention are used for controlling pests from the family Isoptera.

In a preferred embodiment of the invention, the methods of the present invention are used for controlling pests from the family Diptera.

In a preferred embodiment of the invention, the methods of the present invention are used for controlling pests from the family Thipidae.

### Mixtures for Soil and Seed Treatment Application Methods

One embodiment of the present invention is the use of compounds of formula (I) in soil application methods.

One embodiment of the present invention is the use of mixtures of compounds of formula (I) with one or more pesticidal compound(s) (II) in soil application methods.

One embodiment of the present invention is the use of compounds of formula (I) in seed treatment methods.

One embodiment of the present invention is the use of mixtures of compounds of formula (I) with one or more pesticidal compound(s) (II) in seed treatment methods.

With regard to the use in the soil application and seed treatment methods of the present invention, preferably compounds of formula (I) is applied alone or in combination with preferred pesticidal compounds (II), wherein the compounds (II) are selected as defined hereinbelow.

In one preferred embodiment of the present invention, the mixtures used in soil application and seed treatment methods are mixtures of compounds of formula (I) with carbamate compounds. In one more preferred embodiment of the present invention, the mixtures used in soil application and seed treatment methods are mixtures of compounds of formula (I) with carbamate compounds, such as mixtures of compounds of formula (I) with carbosulfan.

Especially preferred are such mixtures of compounds of formula (I) with carbosulfan.

In one preferred embodiment of the present invention, the mixtures used in soil application and seed treatment methods are mixtures of compounds of formula (I) with pyrethroid compounds. In one more preferred embodiment of the present invention, the mixtures used in soil application and seed treatment methods are mixtures of compounds of formula (I) with pyrethroid compounds, such as mixtures of compounds of formula (I) with bifenthrin, cyfluthrin, lambda-cyhalothrin, cypermethrin, beta-cypermethrin, deltamethrin, ethofenprox, fenpropathrin, fenvalerate, permethrin, phenothrin or silafluofen;

In one mostly preferred embodiment of the present invention, the mixtures used in soil application and seed treatment methods are such mixtures of compounds of formula (I) with pyrethroid compounds, wherein compounds of formula (I) is combined with bifenthrin, lambda-cyhalothrin, cypermethrin, beta-cypermethrin or deltamethrin;

In one especially preferred embodiment of the present invention, the mixtures used in soil application and seed treatment methods are such mixtures of compounds of formula (I) with pyrethroid compounds, wherein compounds of formula (I) is combined with bifenthrin, lambda-cyhalothrin or cypermethrin.

Especially preferred are such mixtures of compounds of formula (I) with lambda-cyhalothrin.

In one preferred embodiment of the present invention, the mixtures used in soil application and seed treatment methods are mixtures of compounds of formula (I) with nicotinic receptor agonists/antagonists compounds.

In one more preferred embodiment of the present invention, the mixtures used in soil application and seed treatment methods are mixtures of compounds of formula (I) with nicotinic receptor agonists/antagonists compounds, such as mixtures of compounds of formula (I) with acetamiprid, clothianidin, dinotefuran, imidacloprid, thiamethoxam, nitenpyram, spinosad, spinetoram or thiacloprid.

Especially preferred are such mixtures of compounds of formula (I) with acetamiprid, more preferably a compound of formula IA, also preferably a compound of formula IB, also preferably a compound of formula IC, also preferably a compound of formula ID; more preferably a compound selected from the compounds I-1 to I-40 as defined in Table C; more preferably a compound selected from compounds I-11, I-16, I-21, I-26, I-31 according to Table C/C′

Especially preferred are such mixtures of compounds of formula (I) with chlothianidin, more preferably a compound of formula IA, also preferably a compound of formula IB, also preferably a compound of formula IC, also preferably a compound of formula ID; more preferably a compound selected from the compounds I-1 to I-40 as defined in Table C; more preferably a compound selected from compounds I-11, I-16, I-21, I-26, I-31 according to Table C/C′.

Especially preferred are such mixtures of compounds of formula (I) with dinotefuran, more preferably a compound of formula IA, also preferably a compound of formula IB, also preferably a compound of formula IC, also preferably a compound of formula ID; more preferably a compound selected from the compounds I-1 to I-40 as defined in Table C; more preferably a compound selected from compounds I-11, I-16, I-21, I-26, I-31 according to Table C/C′.

Especially preferred are such mixtures of compounds of formula (I) with imidacloprid, more preferably a compound of formula IA, also preferably a compound of formula IB, also preferably a compound of formula IC, also preferably a compound of formula ID; more preferably a compound selected from the compounds I-1 to I-40 as defined in Table C; more preferably a compound selected from compounds I-11, I-16, I-21, I-26, I-31 according to Table C/C′.

Especially preferred are such mixtures of compounds of formula (I) with thiamethoxam, more preferably a compound of formula IA, also preferably a compound of formula IB, also preferably a compound of formula IC, also preferably a compound of formula ID; more preferably a compound selected from the compounds I-1 to I-40 as defined in Table C; more preferably a compound selected from compounds I-11, I-16, I-21, I-26, I-31 according to Table C/C′.

Especially preferred are such mixtures of compounds of formula (I) with thiacloprid, more preferably a compound of formula IA, also preferably a compound of formula IB, also preferably a compound of formula IC, also preferably a compound of formula ID; more preferably a compound selected from the compounds I-1 to I-40 as defined in Table C; more preferably a compound selected from compounds I-11, I-16, I-21, I-26, I-31 according to Table C/C′.

Especially preferred are such mixtures of compounds of formula (I) with sulfoxaflor, more preferably a compound of formula IA, also preferably a compound of formula IB, also preferably a compound of formula IC, also preferably a compound of formula ID; more preferably a compound selected from the compounds I-1 to I-40 as defined in Table C; more preferably a compound selected from compounds I-11, I-16, I-21, I-26, I-31 according to Table C/C′.

Especially preferred are such mixtures of compounds of formula (I) with fipronil, more preferably a compound of formula IA, also preferably a compound of formula IB, also preferably a compound of formula IC, also preferably a compound of formula ID; more preferably a compound selected from the compounds I-1 to I-40 as defined in Table C; more preferably a compound selected from compounds I-11, I-16, I-21, I-26, I-31 according to Table C/C′.

Especially preferred are such mixtures of compounds of formula (I) with indoxacarb, more preferably a compound of formula IA, also preferably a compound of formula IB, also preferably a compound of formula IC, also preferably a compound of formula ID; more preferably a compound selected from the compounds I-1 to I-40 as defined in Table C; more preferably a compound selected from compounds I-11, I-16, I-21, I-26, I-31 according to Table C/C′.

Especially preferred are such mixtures of compounds of formula (I) with bifenthrin, more preferably a compound of formula IA, also preferably a compound of formula IB, also preferably a compound of formula IC, also preferably a compound of formula ID; more preferably a compound selected from the compounds I-1 to I-40 as defined in Table C; more preferably a compound selected from compounds I-11, I-16, I-21, I-26, I-31 according to Table C/C′.

In one highly preferred embodiment of the present invention, the mixtures used in soil application and seed treatment methods are such mixtures of compounds of formula (I) with pyrethroid compounds.

In another highly preferred embodiment of the present invention, the mixtures used in soil application and seed treatment methods are such mixtures in which compounds of formula (I) are combined with acetamiprid, clothianidin, dinotefuran, imidacloprid, thiamethoxam, spinosad, spinetoram or thiacloprid.

In one preferred embodiment of the present invention, the mixtures used in soil application and seed treatment methods are mixtures of compounds of formula (I) with GABA gated chloride channel antagonist compounds.

In one more preferred embodiment of the present invention, the mixtures used in soil application and seed treatment methods are mixtures of compounds of formula (I) with GABA gated chloride channel antagonist compounds, such as mixtures of compounds of formula (I) with fipronil; Especially preferred are such mixtures of compounds of formula (I) with fipronil.

In one preferred embodiment of the present invention, the mixtures used in soil application and seed treatment methods are mixtures of compounds of formula (I) with chloride channel activators.

In one preferred embodiment of the present invention, the mixtures used in soil application and seed treatment methods are mixtures of compounds of formula (I) with chloride channel activators, such as mixtures of compounds of formula (I) with abamectin or emamectin benzoate; Especially preferred are such mixtures of compounds of formula (I) with abamectin. Especially preferred are such mixtures of compounds of formula (I) with emamectin benzoate. Especially preferred are such mixtures of compounds of formula (I) with pymetrozine. Especially preferred are such mixtures of compounds of formula (I) with flonicamid. Especially preferred are such mixtures of compounds of formula (I) with compound II-M.X.2 which is cyclopropaneacetic acid, 1,1′-[(3S,4R,4aR,6S,6aS,12R,12aS,12bS)-4-[[(2-cyclopropylacetyl)oxy]methyl]-1,3,4,4a,5,6,6a,12,12a,12b-decahydro-12-hydroxy-4,6a,12b-trimethyl-11-oxo-9-(3-pyridinyl)-2H,11H-naphtho[2,1-b]pyrano[3,4-e]pyran-3,6-diyl]ester:

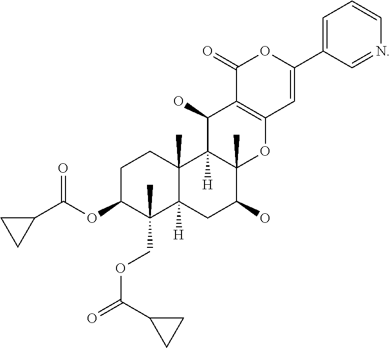

Especially preferred are such mixtures of compounds of formula (I) with buprofezin. Especially preferred are such mixtures of compounds of formula (I) with spirotetramat. Especially preferred are such mixtures of compounds of formula (I) with anthranilamides. Especially preferred are such mixtures of compounds of formula (I) with cyflumetofen. Especially preferred are such mixtures of compounds of formula (I) with cyenopyrafen.

### Extended Mixtures

The mixtures according to the present invention of compounds of formula (I) with pesticidally active compound (II) can optionally also be present together with other additional active substances, e.g. with herbicides, insecticides, growth regulators, fungicides or else with fertilizers or inoculants, as pre-mix or, if appropriate, not until immediately prior to use (tank mix).

The mixture(s) of at least one active compound of formula (I) with at least one active compound II are herein referred to as “mixture(s) according to the invention”.

In a specific embodiment, the mixture according to the invention is a mixture of one active compound of formula (I) with one active compound II (binary mixture).

In another embodiment, the mixture according to the invention is a mixture of one active compound of formula (I) with at least one active compound II.

In another embodiment, the mixture according to the invention is a mixture of one active compound of formula (I) with two active compounds II (ternary mixture).

In another embodiment, the mixture according to the invention is a mixture of one active compound of formula (I) with three active compounds II (4-way mixture).

In another embodiment, the mixture according to the invention is a mixture of one active compound of formula (I) with four active compounds II (5-way mixture).

Thus the present invention relates additionally also to soil application and seed treatment methods, wherein the method comprises the application of and the treatment with a mixture comprising compounds of formula (I), a pesticidal compound (II) as described herein further above, and optionally one or more fungicidal compound(s) (III) selected from groups F.I) to F.XII) listed herein above and/or one or more insecticidal compound(s) (IV) selected from the groups II-M.1 to II-M.X herein above listed further above.

### Preferences of the Extended Mixtures

Additional fungicidal compounds (III) preferably selected for the extended mixtures of the present invention are amisulbrom, azoxystrobin, benalaxyl, bixafen, boscalid, coumethoxystrobin, coumoxystrobin, cyazofamid, cyproconazole, difenoconazole, dimethomorph, dimoxystrobin, ethaboxam, fludioxonil, fluopyram, fluoxastrobin, fluquinconazole, fluxapyroxad, hymexazole, ipconazole, iprodione, isopyrazam, metalaxyl, metconazole, penflufen, penthiopyrad, picoxystrobin, prochloraz, prothioconazole, pyraclostrobin, pyrimethanil, sedaxane, silthiofam, tebuconazole, tebuconazole, thiabendazol, thiophanate methyl, thiram, triadimenol, triasoxide, triazoxide, trifloxystrobin or triticonazole.

The additional insecticidal compounds (IV) for the extended mixtures are selected in analogy to the preferences provided for pesticidal compound (II) listed further above.

Preferably the method for soil application and seed treatment comprises the application of and the treatment with a ternary mixture comprising cycloxaprid (compound I), the pesticidal active compound (II) and as a third component a fungicide as compound (III) selected from groups F.I) to F.XII).

In yet another embodiment, the method comprises the application of and the treatment with a ternary mixture comprising a compound of formula (I), the pesticidal active compound (II) and as a third component an insecticide as compound (IV) selected from the groups II-M.1 to II-M.X.

Preferably the method for soil application and seed treatment comprises the application of and the treatment with quaternary mixture comprising a compound of formula (I), the pesticidally active compound (II) and as third and fourth component two fungicide compounds (III) selected from groups F.I) to F.XII).

In yet another embodiment, the method comprises the application of and the treatment with with a quaternary mixture comprising a compound of formula (I), the pesticidally active compound (II), as a third component a fungicide as compound (III) selected from groups F.I) to F.XII) and as a fourth component an insecticide as compound (IV) selected from the groups II-M.1 to II-M.X.

Preferably the method of soil application and seed treatment comprises the application of and the treatment with quinary mixture comprising a compound of formula (I), the pesticidally active compound (II) and as third, fourth and fifth component three fungicide compounds (III) selected from groups F.I) to F.XII).

In yet another embodiment, the method comprises the application of and the treatment with a quinary mixture comprising a compound of formula (I), the pesticidally active compound (II), as a third and fourth component two fungicide as compounds (III) selected from groups F.I) to F.XII) and as a fifth component an insecticide as compound (IV) selected from the groups II-M.1 to II-M.X.

### EXAMPLES

The present invention is now illustrated in further detail by the following examples.

### A. Chemistry

The compounds I of formula I can be accomplished according to standard methods of organic chemistry, e.g. by the methods or working examples described in WO 2007/006670, PCT/EP2012/065650, PCT/EP2012/065651.

The characterization can be done by coupled High Performance Liquid Chromatography/mass spectrometry (HPLC/MS), by NMR or by their melting points.

Method A: Analytical HPLC column: RP-18 column Chromolith Speed ROD from Merck KgaA (Germany). Elution: acetonitrile+0.1% trifluoroacetic acid (TFA)/water+0.1% trifluoroacetic acid (TFA) in a ratio of from 5:95 to 95:5 in 5 minutes at 40° C.

Method B: Analytical UPLC column: Phenomenex Kinetex 1.7 μm XB-C18 100A; 50×2.1 mm; mobile phase: A: water+0.1% trifluoroacetic acid (TFA); B: acetonitrile+0.1% TFA; gradient: 5-100% B in 1.50 minutes; 100% B 0.20 min; flow: 0.8-1.0 mL/min in 1.50 minutes at 60° C. MS-method: ESI positive.

¹H-NMR. The signals are characterized by chemical shift (ppm) vs. tetramethylsilane, by their multiplicity and by their integral (relative number of hydrogen atoms given). The following abbreviations are used to characterize the multiplicity of the signals: m=multiplett, q=quartett, t=triplett, d=doublet and s=singulett.

### Preparation Examples

log P determinations were performed via capillary electrophorese on a cePro9600™ from CombiSep.

Starting Materials

6,8-dichloro-1H-benzo[d][1,3]oxazine-2,4-dione and 6-chloro-8-methyl-1H-3,1-benzoxazine-2,4-dione were prepared according to WO 2007/43677.

S,S-Diisopropyl-S-aminosulfonium 2,4,6-trimethylphenylsulfonat was prepared according to Y. Tamura et al, Tetrahedron 1975, 31, 3035-3040.

2-(3-Chloropyridin-2-yl)-5-bromo-2H-pyrazole-3-carbonyl chloride was prepared according to WO 2007/24833.

### Preparation Examples P.1 to P.4

### Example P.1

### S,S-Dimethyl sulfinium sulfate

To a solution of sodium methylate (15.76 g of a 30% solution in methanol, 87.54 mmol, 1.100 equiv.) in methanol (60 mL) was added dimethyl sulphide (5.44 g, 6.40 mL, 87.6 mmol, 1.10 equiv.) at −5-0° C. To this mixture was added a pre-cooled solution (−20° C.) of hydroxylamine-O-sulfonic acid (9.00 g, 79.6 mmol) in methanol (60 mL) and the internal temperature was maintained at −5-0° C. After stirring at room temperature overnight, all solids were removed by filtration. The filtrate was concentrated in vacuo and the residue was triturated with acetonitrile (50 mL) to yield the title compound (7.88 g, 39%).

The following compounds were prepared by analogy to example P.1:

S,S-diethyl sulfinium sulfate
S-ethyl-S-isopropyl sulfinium sulfate
S,S-diisopropyl sulfinium sulfate
S,S-bis(2-cyclopropylmethyl) sulfinium sulfate
S,S-bis(2-cyclopropylethyl) sulfinium sulfate
S,S-bis(cyclobutylmethyl) sulfinium sulfate
S,S-bis(cyclopentylmethyl) sulfinium sulfate
S-cyclopropylmethyl-S-ethyl sulfinium sulfate
S-(2-cyclopropylethyl)-S-ethyl sulfinium sulfate
S-(2-cyclopropylethyl)-S-isopropyl sulfinium sulfate
S-(1-cyclopropylethyl)-S-isopropyl sulfinium sulfate
S-cyclobutylmethyl-S-ethyl sulfinium sulfate
S-cyclopentylmethyl-S-ethyl sulfinium sulfate
S-cyclopropylmethyl-S-isopropyl sulfinium sulfate
S-cyclobutylmethyl-S-isopropyl sulfinium sulfate
S-cyclopentylmethyl-S-isopropyl sulfinium sulfate
S,S-di-n-propyl sulfinium sulfate
S-vinyl-S-ethyl sulfinium sulfate

### Example P.2

### 8-Bromo-6-chloro-1H-benzo[d][1,3]oxazine-2,4-dione

To a solution of 2-amino-3-bromo-5-chlorobenzoic acid (10.0 g, 39.9 mmol) in dioxane (170 mL) was added phosgene (20% in toluene, 42.0 mL, 79.9 mmol) over a period of 15 mins. The reaction was stirred at ambient temperature for 48 h and then concentrated in vacuo. The resulting solid was crushed and further dried in vacuo to yield the desired product (12.6 g, 114%) which was used in the subsequent step without further purification.

The following compounds were prepared by analogy to example P.2:
6,8-dichloro-1H-benzo[d][1,3]oxazine-2,4-dione,
6,8-dibromo-1H-benzo[d][1,3]oxazine-2,4-dione,
6-Bromo-8-chloro-1H-benzo[d][1,3]oxazine-2,4-dione,
8-Bromo-6-chloro-1H-benzo[d][1,3]oxazine-2,4-dione,
6-chloro-8-methyl-1H-benzo[d][1,3]oxazine-2,4-dione,
6-bromo-8-methyl-1H-benzo[d][1,3]oxazine-2,4-dione,
6-cyano-8-methyl-1H-benzo[d][1,3]oxazine-2,4-dione,
6-chloro-8-trifluoromethyl-1H-benzo[d][1,3]oxazine-2,4-dione,
8-chloro-6-trifluoromethyl-1H-benzo[d][1,3]oxazine-2,4-dione,
6-bromo-8-trifluoromethyl-1H-benzo[d][1,3]oxazine-2,4-dione,
8-bromo-6-trifluoromethyl-1H-benzo[d][1,3]oxazine-2,4-dione,
8-chloro-6-cyano-1H-benzo[d][1,3]oxazine-2,4-dione,
6-chloro-8-methoxy-1H-benzo[d][1,3]oxazine-2,4-dione,
6-chloro-8-cyclopropyl-1H-benzo[d][1,3]oxazine-2,4-dione,
6-chloro-8-ethyl-1H-benzo[d][1,3]oxazine-2,4-dione,
6-difluoromethoxy-8-methyl-1H-benzo[d][1,3]oxazine-2,4-dione,
6-cyano-8-methoxy-1H-benzo[d][1,3]oxazine-2,4-dione,
6-fluoro-8-methyl-1H-benzo[d][1,3]oxazine-2,4-dione,
6-iodo-8-methyl-1H-benzo[d][1,3]oxazine-2,4-dione,
6-nitro-8-methyl-1H-benzo[d][1,3]oxazine-2,4-dione,
6-(5-chloro-2-thienyl)-8-methyl-1H-benzo[d][1,3]oxazine-2,4-dione,
6-(3-pyrazol-1H-yl)-8-methyl-1H-benzo[d][1,3]oxazine-2,4-dione,
6-(3-isoxazolyl)-8-methyl-1H-benzo[d][1,3]oxazine-2,4-dione,
6-(hydroxyiminomethyl)-8-methyl-1H-benzo[d][1,3]oxazine-2,4-dione,
6-(methoxyiminomethyl)-8-methyl-1H-benzo[d][1,3]oxazine-2,4-dione,
6-(dimethylhydrazonomethyl)-8-methyl-1H-benzo[d][1,3]oxazine-2,4-dione and
6-(2,2,2-trifluoroethylhydrazonomethyl)-8-methyl-1H-benzo[d][1,3]oxazine-2,4-dione.

### Example P.3

### 1-(3-chloro-2-pyridyl)-3-trifluoromethyl-1H-pyrazol

a) 2.71 kg of 1,1,1-trifluoro-4-methoxy-but-3-en-2-one, 2.44 kg of ethanol and 3.10 kg of water were charged into a reaction vessel. 20 ml of concentrated hydrochloric acid and 0.80 kg of hydrazine hydrate were successively added and the mixture was heated to reflux for 4 h. The mixtures was allowed to cool and neutralized by addition of 10% aqueous NaOH to about pH 4-5. Then the mixture was evaporated. Toluene was added and the mixture was again evaporated to yield 2 kg of raw 3-trifluoromethylpyrazole with a purity of >85%.

b) 1.72 kg (10.75 mol) of the raw 3-trifluoromethylpyrazole obtained in step a), 1.75 kg (11.83 mol) of 2,3-dichloropyridine and 4.73 kg of dimethyl formamide were charged to a reaction vessel. 2.97 kg (21.50 mol) of potassium carbonate were added, the mixture was heated to 120° C. with stirring and kept at 120-125° C. for further 3 h. The reaction mixtures was cooled to 25° C. and poured into 20 l of water. The thus obtained mixture was extracted twice with 5 L of tert.-butylmethyl ether. The combined organic phases were washed with 4 l of water and then evaporated to dryness. Toluene was added and the mixture was again evaporated to dryness. Thereby, the 2.7 kg of the title compound was obtained (purity >75% as determined by GC; yield 81.5%). The product can be purified by distillation.

¹H-NMR (400 MHz, CDCl₃): δ [delta]=6.73 (d, 1H), 7.38 (d, 1H), 7.95 (m, 1H), 8.14 (m, 1H), 8.46 (m, 1H).

### Example P.4

### 2-(3-Chloropyridin-2-yl)-5-trifluoromethyl-2H-pyrazole-3-carbonyl chloride

In a reaction vessel equipped with a thermometer, septum, nitrogen inlet and stirring bar, 10.0 g (40.4 mmol) of 1-(3-chloro-2-pyridyl)-3-trifluoromethyl-1H-pyrazole were dissolved in 50 ml of dry dimethoxyethane. By means of a syringe, 40.4 ml of a 2 M solution (80.8 mmol, 2.0 equiv.) of isopropyl magnesium chloride in tetrahydrofuran were added dropwise with stirring, while cooling the vessel with an ice bath and keeping the internal temperature at about 5° C. The mixture was stirred for further 2 hours at 5° C. Then the ice-bath was removed and carbon dioxide was bubbled through mixture causing an increase of the temperature up to 28° C. After 10 minutes, the exothermic reaction has ceased, and, the mixture was cooled and all volatiles were removed by evaporation. The residue containing the carboxylate compound I-A was taken up in 50 mL of dichloromethane and one drop of dry DMF was added. To this mixture, 14.41 g (121.2 mmol, 3.0 equiv.) of thionyl chloride were added and heated to reflux for 3 hours. After cooling, the resulting precipitate was removed by filtration and the mother liquid was concentrated in vacuum to obtain 13.0 g of the title compound (purity >85%, yield 100%) which was used in the next step without further purification.

¹H-NMR (400 MHz, CDCl₃): δ [delta]=7.43-7.54 (m, 2H), 7.93 (d, 1H), 8.52 (m, 1H).

### Example P.5

### 2-amino-5-chloro-N-(dimethyl-λ⁴-sulfanylidene)-3-methyl-benzamide

To a solution of 6-chloro-8-methyl-1H-3,1-benzoxazine-2,4-dione (3.00 g, 12.8 mmol) in dichloromethane (40 mL) was added dimethyl sulfinium sulfate (2.25 g, 8.93 mmol, 0.70 equiv.) and potassium tert-butylate (1.58 g, 14.0 mmol, 1.10 equiv.) at room temperature. The mixture was stirred for 1.5 h, upon which water was added and the layers were separated. The aqueous layer was extracted with dichloromethane, combined organic layers were dried over sodium sulphate and concentrated in vacuo. The residue was purified by flash-chromatography on silica gel to yield the title compound (2.63 g, 84%).

Characterization by HPLC-MS: 1.855 min, M=245.00.

### Example P.6

### 2-amino-5-chloro-N-(bis-2-methylpropyl-λ⁴-sulfanylidene)-3-methyl-benzamide

To a solution of 6-chloro-8-methyl-1H-3,1-benzoxazine-2,4-dione (3.00 g, 12.8 mmol) in dichloromethane (40 mL) was added bis-2-methylpropyl sulfinium sulfate (3.76 g, 8.93 mmol, 0.70 equiv.) and potassium tert-butylate (1.58 g, 14.0 mmol, 1.10 equiv.) at room temperature. The mixture was stirred for 1.5 h, upon which water was added and the layers were separated. The aqueous layer was extracted with dichloromethane, combined organic layers were dried over sodium sulphate and concentrated in vacuo. The residue was purified by flash-chromatography on silica gel to yield the title compound (2.89 g, 69%).

Characterization by ¹H-NMR (400 MHz, DMSO-d₆): δ [delta]=1.04 (m, 12H), 2.06 (s, 3H), 2.96 (m, 2H), 3.01 (m, 2H), 6.62 (br. s, 2H), 7.03 (s, 1H), 7.72 (s, 1H).

### Example P.7

### 2-amino-5-chloro-N-(diethyl-λ⁴-sulfanylidene)-3-methyl-benzamide

To a solution of 6-chloro-8-methyl-1H-3,1-benzoxazine-2,4-dione (2 g, 0.01 mol) in anhydrous propylene carbonate (30 mL) was added bis-2-ethyl sulfinium sulfate (2.04 g, 0.01 mol, 0.70 equiv.) and triethyl amine (1.38 mL, 1.0 g g, 0.01 mol, 1.05 equiv.) at room temperature. The mixture was stirred for 4.5 h, and then added dropwise to ice-water. The mixture was extracted with dichloromethane and the combined organic layers were dried over sodium sulphate and concentrated in vacuo. The residue was triturated with ether to yield the title compound (1.43 g, 55%).

Characterization by ¹H-NMR (400 MHz, CDCl₃): δ [delta]=1.39 (t, 6H), 2.13 (s, 3H), 3.02 (q, 4H), 5.95 (br. S, 2H), 7.01 (s, 1H), 7.98 (s, 1H).

### Example P.8

### 2-amino-3,5-dichloro-N-(bis-2-methylpropyl-λ⁴-sulfanylidene)-benzamide

The title compound was prepared by analogy to the method of example P.6

Yield: 60%

Characterization by ¹H-NMR (400 MHz, DMSO-d₆): δ [delta]=1.23 (d, 6H), 1.38 (d, 6H), 3.42 (m, 2H), 7.02 (br. s, 2H), 7.41 (s, 1H), 7.95 (s, 1H).

### Example P.9

### 2-amino-3,5-dibromo-N-(bis-2-methylpropyl-λ⁴-sulfanylidene)-benzamide

The title compound was prepared by analogy to the method of example P.6

Yield: 66%

Characterization by HPLC-MS: 3.409 min, m/z=410.90 (Method A)

### Preparation of the Compounds of Formula IA-1

### Examples 1 to 4

Example 1: 2-(3-chloro-2-pyridyl)-N-[2,4-dichloro-6-[(diethyl-λ⁴-sulfanylidene)carbamoyl]phenyl]-5-(trifluoromethyl)pyrazole-3-carboxamide (Compound I-16) To a suspension of potassium carbonate (8.08 g, 58.5 mmol, 1.50 equiv) and 2-amino-3,5-dichloro-N-(diethyl-λ⁴-sulfanylidene)benzamide (11.43 g, 38.98 mmol) in acetonitrile (100 mL) was added a solution of 2-(3-chloro-2-pyridyl)-5-(trifluoromethyl)pyrazole-3-carbonyl chloride (15.8 g, 43.31 mmol, 1.10 equiv.) in acetonitrile (50 mL) at room temperature. After 6 h at this temperature, the solids were filtered off. The resulting filtrate was washed with water and dried over Na₂SO₄. After filtration, the filtrate was concentrated in vacuum and the resulting solids were crystallized from diisopropyl ether to yield the title compound (19.53 g, 88%).

Characterization by ¹H-NMR (400 MHz, DMSO-d₆):

δ [delta]=1.13 (t, 6H), 2.91 (m, 2H), 3.08 (m, 2H), 7.67 (dd, 1H), 7.77 (s, 2H), 7.89 (s, 1H), 8.22 (d, 1H), 8.51 (d, 1H), 10.73 (s, 1H).

### Example 2

### Synthesis of 2-(3-chloro-2-pyridyl)-N-[2,4-dichloro-6-[(bis-2-propyl-λ⁴-sulfanylidene)carbamoyl]phenyl]-5-(trifluoromethyl)pyrazole-3-carboxamide (Compound (I-26)

To a suspension of potassium carbonate (0.892 g, 6.46 mmol, 1.10 equiv) and 2-amino-3,5-dichloro-N-(bis-2-propyl-λ⁴-sulfanylidene)benzamide (2.05 g, 5.87 mmol) in toluene (30 mL) was added a solution of 2-(3-chloro-2-pyridyl)-5-(trifluoromethyl)pyrazole-3-carbonyl chloride (2.02 g, 5.87 mmol, 1.00 equiv.) in toluene (20 mL) at 60° C. After 45 min at this temperature, the mixture was cooled and water was added. The resulting precipitate was collected by filtration, washed with water and toluene and dried to obtain the title compound (3.07 g, 84%).

Characterization by HPLC-MS: 1.395 min, M=602.1 (Method B)

Characterization by ¹H-NMR (400 MHz, DMSO-d₆):

δ [delta]=1.18 (d, 6H), 1.22 (d, 6H), 3.30 (m, 2H), 7.68 (dd, 1H), 7.75 (m, 2H), 7.81 (s, 1H), 8.21 (d, 1H), 8.54 (d, 1H), 10.76 (s, 1H).

### Example 3

### Synthesis of 2-(3-chloro-2-pyridyl)-N-[2-methyl-4-chloro-6-[(bis-2-propyl-λ⁴-sulfanylidene)carbamoyl]phenyl]-5-(trifluoromethyl)pyrazole-3-carboxamide (Compound I-21)

To a suspension of potassium carbonate (126.01 g, 911.76 mmol, 1.30 equiv) and 2-amino-3-methyl-5-chloro-N-(bis-2-propyl-λ⁴-sulfanylidene)benzamide (211 g, 701 mmol) in dichloromethane (300 mL) was added a solution of 2-(3-chloro-2-pyridyl)-5-(trifluoromethyl)pyrazole-3-carbonyl chloride (256.78 g, 771.49 mmol, 1.10 equiv.) in dichloromethane (200 mL) at room temperature. After 2 h at this temperature, the solids were filtered off. The resulting filtrate was washed with water and dried over Na₂SO₄. After filtration, the filtrate was concentrated in vacuum and the resulting solids were crystallized from diisopropyl ether to yield the title compound (344.2 g, 85%).

Characterization by HPLC-MS: 1.303 min, M=574.3 (Method B)

Characterization by ¹H-NMR (400 MHz, DMSO-do): δ [delta]=1.20 (d, 6H), 1.30 (d, 6H), 2.15 (s, 3H), 3.30 (m, 2H), 7.41 (s, 1H), 7.62 (m, 2H), 7.80 (s, 1H), 8.22 (d, 1H), 8.52 (d, 1H), 10.88 (s, 1H).

### Example 4a

### 2-(3-chloro-2-pyridyl)-N-[2-methyl-4-chloro-6-[(diethyl-λ⁴-sulfanylidene)carbamoyl]phenyl]-5-(trifluoromethyl)pyrazole-3-carboxamide (Compound I-11)

To a suspension of potassium carbonate (0.71 g, 10 mmol, 1.3 equiv) and 2-amino-3-methyl-5-chloro-N-(diethyl-λ⁴-sulfanylidene)benzamide (1.42 g, 3.96 mmol) in propylene carbonate (20 mL) was added a solution of 2-(3-chloro-2-pyridyl)-5-(trifluoromethyl)pyrazole-3-carbonyl chloride (1.35 g, 4.35 mmol, 1.10 equiv.) in propylene carbonate (10 mL) at room temperature. After 24 h at this temperature, the mixture was poured onto water and spiked with ethanol under vigorous stirring. The resulting solids were collected by filtration and contained pure title compound (1.57 g, 73%).

Characterization by HPLC-MS: 1.19 min, m/z 546.1 (M+H)⁺; (Method B)

Characterization by ¹H-NMR (500 MHz, DMSO) [delta]: 10.87 (s, 1H), 8.53 (d, 1H), 8.22 (d, 1H), 7.75 (s, 1H), 7.65 (m, 2H), 7.40 (s, 1H), 3.09 (m, 2H), 2.92 (m, 2H) 1.15 (m, 6H).

### Example 4b

### 2-(3-chloro-2-pyridyl)-N-[2-methyl-4-chloro-6-[(diethyl-λ⁴-sulfanylidene)carbamoyl]phenyl]-5-(trifluoromethyl)pyrazole-3-carboxamide (Compound I-11)

To a solution of 2-(3-chloro-2-pyridyl)-5-(trifluoromethyl)pyrazole-3-carbonyl chloride (150 g, 435 mmol) in acetonitrile (900 mL) at room temperature was added potassium carbonate (59 g, 427 mmol). A solution of 2-amino-5-chloro-N-(diethyl-sulfanylidene)-3-methyl-benzamide (117 g, 427 mmol) in acetonitrile (100 mL) was added dropwise within 1 hour while maintaining a reaction temperature of 25-28° C. with occasional cooling (slightly exothermic reaction). The mixture was stirred for 16 hours at room temperature. The reaction mixture was then poured on ice-water mixture (5 L) and the pH was adjusted to 7-8 with concentrated HCl. The mixture stirred for an additional 2 hours. The light brown solid was filtered, washed with water and dried under air to give the crude product (229 g).

3 combined batches of crude product (789 g) were suspended in acetonitrile (2.6 L) and dissolved upon heating at 60° C. After 1 hour of stirring at 60° C. the solution was cooled by means of an ice-bath and the thereby formed solid was filtered off. The mother-liquor was concentrated to 300 mL and cooled with ice-bath. Thereby additional solid formed was filtered. The combined solids were washed with cold acetonitrile and dried at 50° C. in a vacuum-oven over night to give the title product (703 g, 89%) as a crystalline white solid.

By the methods described in examples 1 to 4 or analogy thereof, the compounds of formula (IA-1) summarized in table C were prepared:

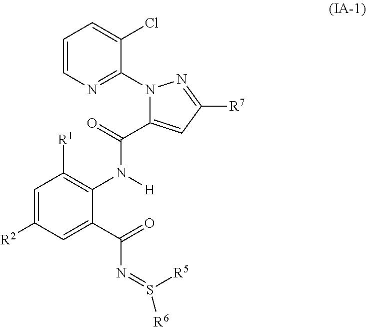

TABLE C

| | R¹ | R² | R⁷ | R⁵ | R⁶ | MS | RT[min] | m/z |
|---|---|---|---|---|---|---|---|---|
| I-1 | Me | Cl | CF₃ | CH₃ | CH₃ | logP: 2.9 [pH = 10.0]; m.p: 182° C. | | |
| I-2 | Me | Cl | CHF₂ | CH₃ | CH₃ | B | 1.06 | 500.2 |
| I-3 | Me | Cl | Br | CH₃ | CH₃ | A | 3.067 | 529.95 |
| I-4 | Me | Cl | Cl | CH₃ | CH₃ | | | |
| I-5 | Me | Cl | CN | CH₃ | CH₃ | | | |
| I-6 | Cl | Cl | CF₃ | CH₃ | CH₃ | A | 3.372 | 539.95 |
| I-7 | Cl | Cl | CHF₂ | CH₃ | CH₃ | B | 1.062 | 520.2 |
| I-8 | Cl | Cl | Br | CH₃ | CH₃ | A | 3.015 | 549.80 |
| I-9 | Cl | Cl | Cl | CH₃ | CH₃ | | | |
| I-10 | Cl | Cl | CN | CH₃ | CH₃ | | | |
| I-11 | Me | Cl | CF₃ | C₂H₅ | C₂H₅ | B | 1.207 | 546.1 |
| I-12 | Me | Cl | CHF₂ | C₂H₅ | C₂H₅ | B | 1.134 | 528.2 |
| I-13 | Me | Cl | Br | C₂H₅ | C₂H₅ | A | 3.309 | 557.95 |
| I-14 | Me | Cl | Cl | C₂H₅ | C₂H₅ | | | |
| I-15 | Me | Cl | CN | C₂H₅ | C₂H₅ | B | 1.098 | 503.3 |
| I-16 | Cl | Cl | CF₃ | C₂H₅ | C₂H₅ | A | 3.450 | 565.90 |
| I-17 | Cl | Cl | CHF₂ | C₂H₅ | C₂H₅ | B | 1.144 | 549.9 |
| I-18 | Cl | Cl | Br | C₂H₅ | C₂H₅ | | | |
| I-19 | Cl | Cl | Cl | C₂H₅ | C₂H₅ | | | |
| I-20 | Cl | Cl | CN | C₂H₅ | C₂H₅ | B | 1.119 | 524.9 |
| I-21 | Me | Cl | CF₃ | CH(CH₃)₂ | CH(CH₃)₂ | B | 1.303 | 574.3 |
| I-22 | Me | Cl | CHF₂ | CH(CH₃)₂ | CH(CH₃)₂ | B | 1.225 | 556.3 |
| I-23 | Me | Cl | Br | CH(CH₃)₂ | CH(CH₃)₂ | logP: 2.9 [pH = 10.0] | | |
| I-24 | Me | Cl | Cl | CH(CH₃)₂ | CH(CH₃)₂ | | | |
| I-25 | Me | Cl | CN | CH(CH₃)₂ | CH(CH₃)₂ | B | 1.19 | 531.3 |
| I-26 | Cl | Cl | CF₃ | CH(CH₃)₂ | CH(CH₃)₂ | A | 3.835 | 596.05 |
| I-27 | Cl | Cl | CHF₂ | CH(CH₃)₂ | CH(CH₃)₂ | B | 1.24 | 578 |
| I-28 | Cl | Cl | Br | CH(CH₃)₂ | CH(CH₃)₂ | A | 3.538 | 605.80 |
| I-29 | Cl | Cl | Cl | CH(CH₃)₂ | CH(CH₃)₂ | | | |
| I-30 | Cl | Cl | CN | CH(CH₃)₂ | CH(CH₃)₂ | B | 1.209 | 553.1 |
| I-31 | Br | Br | CF₃ | C₂H₅ | C₂H₅ | B | 1.218 | 655.9 |
| I-32 | Br | Br | CHF₂ | C₂H₅ | C₂H₅ | B | 1.171 | 638.1 |
| I-33 | Br | Br | Br | C₂H₅ | C₂H₅ | | | |
| I-34 | Br | Br | Cl | C₂H₅ | C₂H₅ | | | |
| I-35 | Br | Br | CN | C₂H₅ | C₂H₅ | | | |
| I-36 | Br | Br | CF₃ | CH(CH₃)₂ | CH(CH₃)₂ | A | 3.665 | 683.90 |
| I-37 | Br | Br | CHF₂ | CH(CH₃)₂ | CH(CH₃)₂ | B | 1.245 | 666.1 |
| I-38 | Br | Br | Br | CH(CH₃)₂ | CH(CH₃)₂ | | | |
| I-39 | Br | Br | Cl | CH(CH₃)₂ | CH(CH₃)₂ | | | |
| I-40 | Br | Br | CN | CH(CH₃)₂ | CH(CH₃)₂ | | | |

### B. Biology

### B.1.1 Soil Drench Assay in Lima Bean

Test solution comprising a compound of the present invention is prepared at desired concentration using water and an organic solvent. Potted lima bean plants are treated with test solution by means of soil drenching. After the desired time, a mixed population of two spotted spider mites is released onto the leaves.

After the desired time after the release of spider mites, the acaricidal efficacy is measured by means of the rating of the damage caused by spider mites or the spider mite mortality.

### B.1.2 Seed Treatment Assay in Cotton

Test solution comprising a compound of the present invention is prepared at desired concentration using water and an organic solvent. Cotton seeds are coated with such prepared test solution and sown to the pots. After plant emergence, a mixed population of two spotted spider mites is released onto the leaves.

After the desired time after the release of spider mites, the acaricidal efficacy is measured by means of the rating of the damage caused by spider mites or the spider mite mortality.

### B.1.3 Seed Treatment Assay in Cucumber

Test solution comprising a compound of the present invention is prepared at desired concentration using water and an organic solvent. Cucumber seeds are coated with such prepared stest olution and sown to the pots. After plant emergence, a mixed population of two spotted spider mites is released onto the leaves.

After the desired time after the release of spider mites, the acaricidal efficacy is measured by means of the rating of the damage caused by spider mites or the spider mite mortality.

B. 1.4 Soil Incorporation Against Western Corn Rootworm (Diabrotica virgifera virgifera)

The active compound was applied in acetone at rates of 5 and 50 ppm a.i./soil (w/w). Treatments were applied in solution to sifted, North Carolina loamy sand (Sandhill soil) in a plastic bag. Treatments were thoroughly incorporated by sealing and shaking each bag by hand and allowing the solution to soak through the soil mass for at least 10 minutes before unsealing. The bags were then kept open in a fume hood overnight to evaporate the solvent from the soil.

One day after treatment (DAT) distilled water for moisture and water-soaked millet seed (Panicum miliaceum ‘white millet’) as a food source were added to each bag and mixed in thoroughly. 11 cm³ of millet and soil mixture were dispensed into a 1 oz. plastic cup. Each cup was infested with 10 western corn rootworm second-instar larvae. Each cup or group of four cells was a replicate, and replication was 3×. The test was maintained in incubators at 26° C. in the dark. Mortality was evaluated 3 days after infestation (DAI) and mean percent mortality was calculated.

In this test, compounds I-11, I-16, I-21, I-26 at 50 ppm showed over 70% mortality in comparison with untreated controls.

B. 1.5 Soil Incorporation Against Black Cutworm (Agrotis ipsilon)

The active compound was applied in acetone at rates of 5 and 50 ppm a.i./soil (w/w). Treatments were applied in solution to sifted, North Carolina loamy sand (Sandhill soil) in a plastic bag. Treatments were thoroughly incorporated by sealing and shaking each bag by hand and allowing the solution to soak through the soil mass for at least 10 minutes before unsealing. The bags were then kept open in a fume hood overnight to evaporate the solvent from the soil. One day after treatment (DAT) distilled water for moisture and water-soaked millet seed (Panicum miliaceum ‘white millet’) as a food source were added to each bag and mixed in thoroughly. 11 cm³ of millet and soil mixture were dispensed into a 1 oz. plastic cup. Each cup was infested with one black cutworm second-instar larva. Each cup or group of four cells was a replicate, and replication was 3×. The test was maintained in incubators at 26° C. with 14 hours. Mortality was evaluated 3 days after infestation (DAI) and mean percent mortality relative to the solvent blank was calculated.

In this test, compounds I-11, I-16, I-21, I-26 at 5 ppm showed over 70% mortality in comparison with untreated controls.

Each cup was infested with 10 western corn rootworm second-instar larvae, and each cell was infested with one black cutworm second-instar larva. Each cup or group of four cells was a replicate, and replication was 3×. The test was maintained in incubators at 26° C. in the dark for western corn rootworm and at 26° C. with 14 hours light for black cutworm. Mortality was evaluated 3 days after infestation (DAI) and mean percent mortality relative to the solvent blank was calculated.

B.2.1 Root Length in Treatment Against Seedcorn Maggot (Anthomyiidae: Delia platura).

The compounds according to the invention and other diamide compounds (cyantraniliprole and chlorantraniliprole) were tested for activity against seedcorn maggot. The compound was dissolved in acetone, and then water was added to achieve a final concentration of 0.5% acetone. Rates were 1 and 10 ppm. Four cucumber seeds (Cucumis sativus ‘National Pickling’) were placed in a germination pouch and 18 ml of solution was added. Pouches were held upright in an incubator (22° C., 14 L:10 D). At 2 days after treatment (DAT), approximately 50 seedcorn maggot eggs were applied to each germination pouch in 0.5 ml of distilled water. Root length of each cucumber plant was measured 7 days after infestation (DAI). Five replicates (pouches) were prepared for each treatment. Analysis of variance was conducted, and mean separation was performed using Student-Newman-Keul's HSD (α=0.05). Percent control was calculated as the mean root length relative to that of the infested and uninfested solvent blank treatments.

TABLE EXP-B.2.1
Root length of cucumbers after treatment with compounds and infestation
by seedcorn maggot. (Pest: Delia platura, eggs)

| | | | % Control |
|---|---|---|---|
| | | Mean Root | (Relative to infested |
| | | Length | and uninfested solvent |
| | | (cm) | blank) |
| | Rate | 9 days after treatment, 7 days after | |
| Treatment | (ppm) | infestation | |
| I-16 | 1 | 11.0 | 100% |
| Cyantraniliprole | 1 | 7.1 | 19% |
| | 10 | 10.3 | 93% |
| Chlorantraniliprole | 1 | 8.4 | 49% |
| | 10 | 9.0 | 63% |
| Infested Solvent | — | 6.3 | 0% |
| Blank (0.5% acetone) | | | |
| Uninfested Solvent | — | 10.6 | 100% |
| Blank (0.5% acetone) | | | |

The compounds tested showed an increased root length and therefore protection from feeding damage by soil pests.

B.2.2 Plant Emergence, Shoot Height and Root Mass in Treatment Against Western Corn Rootworm (Chrysomelidae: Diabrotica virgifera virgifera).

The compounds according to the invention, in formulated form, and other diamide compounds (chlorantraniliprole: Altacor® and Coragen®) were tested for activity against western corn rootworm. Pots were filled with soil mixture (1:1 loamy sand:sand) and watered prior to treatment and planting. Formulations were diluted in distilled water and then applied to 20 g corn seed in a volume of 188 μl in a Hege 11 liquid seed treater and spun for 30 s. One seed was planted per pot 1-3 days after treatment. Five replicates (pots) were prepared for each treatment. Pots were arranged in a randomized complete block design in the greenhouse and top watered daily. At 5-6 days after planting (DAP), 12 western corn rootworm larvae (2nd instar) were infested in each pot. After infestation, pots were maintained in a growth chamber (26° C., 10 hours light:14 hours dark) and bottom-watered as needed. Plant emergence and shoot phytotoxicity were evaluated 5 DAP. Shoot height and fresh root mass were evaluated 7 days after infestation (DAI). Analysis of variance was conducted, and mean separation was performed using Student-Newman-Keul's HSD (α=0.05).

TABLE EXP-B.2.2-1
Plant emergence, shoot height and root mass of corn as corn seed treatment against
western corn rootworm. (Pest: Diabrotica virgifera virgifera, second-instar)

| | | | | % Control (Based on | | % Control (Based on |
|---|---|---|---|---|---|---|
| | | Plant | | shoot height relative | | root mass relative |
| | | Emergence (%) | Mean Shoot | to infested and | Mean Root | to infested and |
| | Rate | 5 days after | Height (cm) | uninfested control) | Mass (g) | uninfested control) |
| Treatment | (g ai/kg seed) | planting | 13 days after planting, 7 days after infestation | | | |
| Infested Solvent | . | 100 | 23.5 | 0 | 0.471 | 0 |
| Blank | | | | | | |
| I-11 | 0.5 | 100 | 26.7 | 36 | 0.680 | 100 |
| | 1.0 | 100 | 33.8 | 100 | 0.678 | 100 |
| I-16 | 0.5 | 100 | 30.2 | 76 | 0.541 | 37 |
| | 1.0 | 100 | 28.6 | 58 | 0.547 | 40 |
| I-21 | 0.5 | 100 | 27.8 | 49 | 0.688 | 100 |
| | 1.0 | 100 | 29.6 | 69 | 0.701 | 100 |
| Altacor | 0.5 | 100 | 17.9 | 0 | 0.428 | 0 |
| 35% WG | 1.0 | 100 | 18.2 | 0 | 0.421 | 0 |
| (chlorantraniliprole) | | | | | | |
| Uninfested | . | 90 | 32.3 | 100 | 0.661 | 100 |
| Solvent Blank | | | | | | |

The compounds tested showed an increased root length and root mass and therefore protection from feeding damage by soil pests.

TABLE EXP-B.2.2-2
Plant emergence, shoot height and root mass of corn as corn seed treatment against
western corn rootworm. (Pest: Diabrotica virgifera virgifera, second-instar)

| | | | | % Control (Based on | | % Control (Based on |
|---|---|---|---|---|---|---|
| | | Plant | | shoot height relative | | root mass relative |
| | | Emergence (%) | Mean Shoot | to infested and | Mean Root | to infested and |
| | Rate | 5 days after | Height (cm) | uninfested control) | Mass (g) | uninfested control) |
| Treatment | (g ai/kg seed) | planting | 12 days after planting, 7 days after infestation | | | |
| Infested Solvent | . | 100 | 21.7 | 0 | 0.445 | 0 |
| Blank | | | | | | |
| I-11 | 0.5 | 100 | 27.3 | 49 | 0.490 | 12 |
| | 1.0 | 100 | 33.8 | 100 | 0.631 | 48 |
| I-16 | 0.5 | 100 | 35.3 | 100 | 0.590 | 38 |
| | 1.0 | 100 | 31.2 | 83 | 0.705 | 68 |
| I-21 | 0.5 | 100 | 31.6 | 86 | 0.570 | 33 |
| | 1.5 | 100 | 31.2 | 83 | 0.598 | 40 |
| Coragen | 0.5 | 100 | 26.1 | 38 | 0.545 | 26 |
| 35% WG | 1.0 | 100 | 26.2 | 39 | 0.578 | 35 |
| (chlorantraniliprole) | 1.5 | 100 | 26.9 | 45 | 0.550 | 27 |
| Uninfested | . | 90 | 33.2 | 100 | 0.829 | 100 |
| Solvent Blank | | | | | | |

The compounds tested showed an increased root length and root mass and therefore protection from feeding damage by soil pests.

1-25. (canceled)

26. A method for controlling or combating insects, arachnids or nematodes, or for protecting plants from attack or infestation by insects, arachnids or nematodes comprising contacting the soil or the artificial growth substrate in which the plant is growing, or plant propagation material from which plants are grown with at least one pesticidally active anthranilamide compound of formula (I): 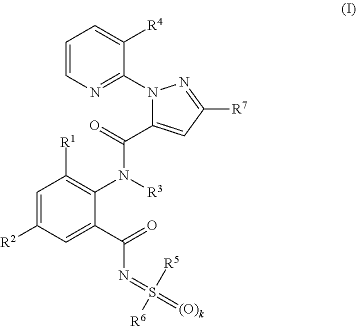
wherein
R¹ is selected from the group consisting of halogen, methyl and halomethyl;
R² is selected from the group consisting of hydrogen, halogen, halomethyl and cyano;
R³ is selected from hydrogen, C₁-C₆ alkyl, C₁-C₆ haloalkyl, C₂-C₆-alkenyl, C₂-C₆-haloalkenyl, C₂-C₆-alkinyl, C₂-C₆-haloalkinyl, C₃-C₈-cycloalkyl, C₃-C₈-halocycloalkyl, C₁-C₄-alkoxy-C₁-C₄-alkyl, C₁-C₄-haloalkoxy-C₁-C₄-alkyl, C(═O)R^{a}, C(═O)OR^{b} and C(═O)NR^{c}R^{d};
R⁴ is hydrogen or halogen;
R⁵, R⁶ are selected independently of one another from the group consisting of hydrogen, C₁-C₁₀-alkyl, C₃-C₈-cycloalkyl, C₂-C₁₀-alkenyl, C₂-C₁₀-alkynyl, wherein the aforementioned aliphatic and cycloaliphatic radicals may be substituted with 1 to 10 substituents R^{e}, and phenyl, which is unsubstituted or carries 1 to 5 substituents R^{f}; or
R⁵ and R⁶ together represent a C₂-C₇-alkylene, C₂-C₇-alkenylene or C₆-C₉-alkynylene chain forming together with the sulfur atom to which they are attached a 3-, 4-, 5-, 6-, 7-, 8-, 9- or 10-membered saturated, partially unsaturated or fully unsaturated ring, wherein 1 to 4 of the CH₂ groups in the C₂-C₇-alkylene chain or 1 to 4 of any of the CH₂ or CH groups in the C₂-C₇-alkenylene chain or 1 to 4 of any of the CH₂ groups in the C₆-C₉-alkynylene chain may be replaced by 1 to 4 groups independently selected from the group consisting of C═O, C═S, O, S, N, NO, SO, SO₂ and NH, and wherein the carbon and/or nitrogen atoms in the C₂-C₇-alkylene, C₂-C₇-alkenylene or C₆-C₉-alkynylene chain may be substituted with 1 to 5 substituents independently selected from the group consisting of halogen, cyano, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkynyl and C₂-C₆-haloalkynyl; said substituents being identical or different from one another if more than one substituent is present;
R⁷ is selected from the group consisting of bromo, chloro, difluoromethyl, trifluoromethyl, nitro, cyano, OCH₃, OCHF₂, OCH₂F, OCH₂CF₃, S(═O)ₙCH₃, and S(═O)ₙCF₃;
R^{a} is selected from the group consisting of C₁-C₆-alkyl, C₂-C₆-alkenyl, C₂-C₆-alkinyl, C₃-C₈-cycloalkyl, C₁-C₆-alkoxy, C₁-C₆-alkylthio, C₁-C₆-alkylsulfinyl, C₁-C₆-alkylsulfonyl, wherein one or more CH₂ groups of the aforementioned radicals may be replaced by a C═O group, and/or the aliphatic and cycloaliphatic moieties of the aforementioned radicals may be unsubstituted, partially or fully halogenated and/or may carry 1 or 2 substituents selected from C₁-C₄ alkoxy; phenyl, benzyl, pyridyl and phenoxy, wherein the last four radicals may be unsubstituted, partially or fully halogenated and/or carry 1, 2 or 3 substituents selected from C₁-C₆-alkyl, C₁-C₆-haloalkyl, C₁-C₆-alkoxy, C₁-C₆-haloalkoxy, (C₁-C₆-alkoxy)carbonyl, C₁-C₆-alkylamino and di-(C₁-C₆-alkyl)amino,
R^{b} is selected from the group consisting of C₁-C₆-alkyl, C₂-C₆-alkenyl, C₂-C₆-alkinyl, C₃-C₈-cycloalkyl, C₁-C₆-alkoxy, C₁-C₆-alkylthio, C₁-C₆-alkylsulfinyl, C₁-C₆-alkylsulfonyl, wherein one or more CH₂ groups of the aforementioned radicals may be replaced by a C═O group, and/or the aliphatic and cycloaliphatic moieties of the aforementioned radicals may be unsubstituted, partially or fully halogenated and/or may carry 1 or 2 substituents selected from C₁-C₄-alkoxy; phenyl, benzyl, pyridyl and phenoxy, wherein the last four radicals may be unsubstituted, partially or fully halogenated and/or carry 1, 2 or 3 substituents selected from C₁-C₆-alkyl, C₁-C₆-haloalkyl, C₁-C₆-alkoxy, C₁-C₆-haloalkoxy and (C₁-C₆-alkoxy)carbonyl;
R^{c}, R^{d} are, independently from one another and independently of each occurrence, selected from the group consisting of hydrogen, cyano, C₁-C₆-alkyl, C₂-C₆-alkenyl, C₂-C₆-alkinyl, C₃-C₈-cycloalkyl, wherein one or more CH₂ groups of the aforementioned radicals may be replaced by a C═O group, and/or the aliphatic and cycloaliphatic moieties of the aforementioned radicals may be unsubstituted, partially or fully halogenated and/or may carry 1 or 2 radicals selected from C₁-C₄-alkoxy;
C₁-C₆-alkoxy, C₁-C₆-haloalkoxy, C₁-C₆-alkylthio, C₁-C₆-alkylsulfinyl, C₁-C₆-alkylsulfonyl, C₁-C₆-haloalkylthio, phenyl, benzyl, pyridyl and phenoxy, wherein the four last mentioned radicals may be unsubstituted, partially or fully halogenated and/or carry 1, 2 or 3 substituents selected from C₁-C₆-alkyl, C₁-C₆-haloalkyl, C₁-C₆-alkoxy, C₁-C₆ haloalkoxy and (C₁-C₆-alkoxy)carbonyl; or
R^{c} and R^{d}, together with the nitrogen atom to which they are bound, may form a 3-, 4-, 5-, 6- or 7-membered saturated, partially unsaturated or fully unsaturated heterocyclic ring which may additionally contain 1 or 2 further heteroatoms or heteroatom groups selected from N, O, S, NO, SO and SO₂, as ring members, where the heterocyclic ring may optionally be substituted with halogen, C₁-C₄-haloalkyl, C₁-C₄-alkoxy or C₁-C₄-haloalkoxy;
R^{e} is independently selected from the group consisting of halogen, cyano, nitro, —OH, —SH, —SCN, C₁-C₆-alkyl, C₂-C₆-alkenyl, C₂-C₆-alkinyl, C₃-C₈-cycloalkyl, wherein one or more CH₂ groups of the aforementioned radicals may be replaced by a C═O group, and/or the aliphatic and cycloaliphatic moieties of the aforementioned radicals may be unsubstituted, partially or fully halogenated and/or may carry 1 or 2 radicals selected from C₁-C₄ alkoxy;
C₁-C₆-alkoxy, C₁-C₆-haloalkoxy, C₁-C₆-alkylthio, C₁-C₆-alkylsulfinyl, C₁-C₆-alkylsulfonyl, C₁-C₆-haloalkylthio, —OR^{a}, —NR^{c}R^{d}, —S(O)ₙR^{a}, —S(O)ₙNR^{c}R^{d}, —C(═O)R^{a}, —C(═O)NR^{c}R^{d}, —C(═O)OR^{b}, —C(═S)R^{a}, —C(═S)NR^{c}R^{d}, —C(═S)OR^{b}, —C(═S)SR^{b}, —C(═NR^{c})R^{b}, —C(═NR^{c})NR^{c}R^{d}, phenyl, benzyl, pyridyl and phenoxy, wherein the last four radicals may be unsubstituted, partially or fully halogenated and/or carry 1, 2 or 3 substituents selected from C₁-C₆-alkyl, C₁-C₆-haloalkyl, C₁-C₆-alkoxy and C₁-C₆-haloalkoxy; or
two vicinal radicals R^{e} together form a group ═O, ═CH(C₁-C₄-alkyl), ═C(C₁-C₄-alkyl)C₁-C₄-alkyl, ═N(C₁-C₆-alkyl) or ═NO(C₁-C₆-alkyl);
R^{f} is independently selected from the group consisting of halogen, cyano, nitro, —OH, —SH, —SCN, C₁-C₆-alkyl, C₂-C₆-alkenyl, C₂-C₆-alkinyl, C₃-C₈-cycloalkyl, wherein one or more CH₂ groups of the aforementioned radicals may be replaced by a C═O group, and/or the aliphatic and cycloaliphatic moieties of the aforementioned radicals may be unsubstituted, partially or fully halogenated and/or may carry 1 or 2 radicals selected from C₁-C₄ alkoxy;
C₁-C₆-alkoxy, C₁-C₆-haloalkoxy, C₁-C₆-alkylthio, C₁-C₆-alkylsulfinyl, C₁-C₆-alkylsulfonyl, C₁-C₆-haloalkylthio, —OR^{a}, —NR^{c}R^{d}, —S(O)ₙR^{a}, —S(O)ₙNR^{c}R^{d}, —C(═O)R^{a}, —C(═O)NR^{c}R^{d}, —C(═O)OR^{b}, —C(═S)R^{a}, —C(═S)NR^{c}R^{d}, —C(═S)OR^{b}, —C(═S)SR^{b}, —C(═NR^{c})R^{b}, and —C(═NR^{c})NR^{c}R^{d};
k is 0 or 1;
n is 0, 1 or 2;
or a stereoisomer, salt, tautomer or N-oxide, or a polymorphic crystalline form, a co-crystal or a solvate of a compound or a stereoisomer, salt, tautomer or N-oxide thereof.

27. The method according to claim 26, wherein the compound of formula I is combined with one or more other pesticidal active compound(s) II selected from insecticides or fungicides.

28. The method according to claim 26, in which the compound of formula I is a compound of formula IA: 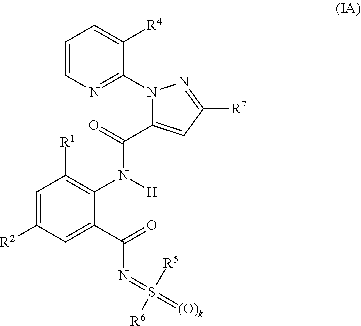
wherein
R⁴ is halogen.

29. The method according to claim 26, in which the compound of formula I is a compound of formula IB: 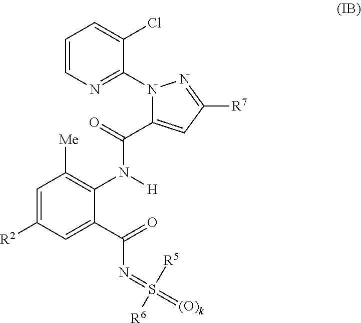
wherein
R² is selected from the group consisting of bromo, chloro, cyano;
R⁷ is selected from the group consisting of bromo, chloro, trifluoromethyl. OCHF₂.

30. The method according to claim 26, in which the compound of formula I is a compound of formula IC: 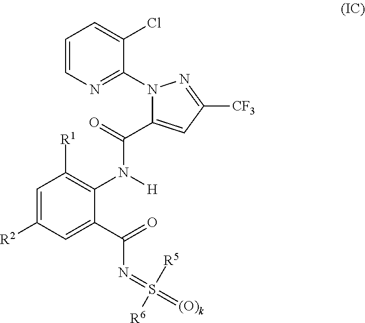
wherein
R¹ is selected from the group consisting of halogen and halomethyl;
R² is selected from the group consisting of bromo, chloro and cyano.

31. The method according to claim 26, in which the compound of formula I is a compound of formula ID: 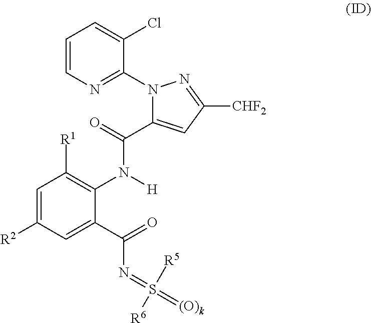
wherein
R¹ is selected from the group consisting of halogen, methyl and halomethyl;
R² is selected from the group consisting of bromo, chloro and cyano.

32. The method according to claim 26, in which in the compound of formula I R⁵ and R⁶ are selected from methyl, ethyl, isopropyl, n-propyl, n-butyl, isobutyl, tert-butyl, cyclopropyl, cyclopropylmethyl.

33. The method according to claim 26, in which in the compound of formula I R⁵ and R⁶ are identical.

34. The method of claim 26, wherein the plant or the plant propagation material to be treated is grown in an artificial growth substrate.

35. The method of claim 34, wherein the artificial growth substrate is selected from rock wool, glass wool, quart sand, gravel, expanded clay and vermiculite.

36. The method claim 26, wherein the plant or plant propagation material to be treated is planted or growing in a closed system.

37. The method of claim 26, wherein the active compound of formula (I) is applied by drenching the soil.

38. The method of claim 26, wherein the active compound of formula (I) is applied by drip irrigation.

39. The method of claim 26, wherein the active compound of formula (I) is applied with drip application systems.

40. The method of claim 26, wherein the active compound of formula (I) is applied by soil injection.

41. The method of according to claim 26, wherein the pesticidally active compound of formula (I) is applied by dipping roots, tubers or bulbs.

42. A method for protection of plant propagation material comprising contacting the plant propagation material with a pesticidally active compound of formula (I) according to claim 26 in pesticidally effective amounts.

43. A method according to claim 44, wherein the plant propagation materials are seeds.

44. A method according to claim 43, wherein the seeds are of transgenic plant.

45. A method according to claim 26, wherein the plants, the plant propagation material or the plant roots and shoots resulting from the treated plant propagation material are protected from the attack by soil pests or foliar pests.

46. Seed treated with a pesticidally active compound of formula (I) according to claim 26 in an amount of from 0.1 g to 10 kg per 100 kg of seeds.

47. The method of claim 26, wherein the plant or the plant propagation material to be treated is selected from the group consisting of vegetables, spices, herbs, ornamentals, conifers, shrubs, cotton, tropical crops, citrus plants, fruits, nuts and grape vines.
